(19) United States
(12) Patent Application Publication
(10) Pub. No.: US 2021/0290056 A1
(43) Pub. Date: Sep. 23, 2021

(54) SYSTEMS AND METHODS FOR CAPTURING, ANNOTATING AND SHARING OPHTHALMIC IMAGES OBTAINED USING A HAND HELD COMPUTER

(71) Applicant: VERANA HEALTH, INC., San Francisco, CA (US)

(72) Inventors: Nitin KARANDIKAR, Cupertino, CA (US); Nikolai STEKLOV, San Francisco, CA (US); Allen JONES, Dublin, CA (US); Douglas N. FOSTER, San Francisco, CA (US); Danghui LIU, Orlando, FL (US); Edwin SARVER, Cookeville, TN (US)

(73) Assignee: VERANA HEALTH, INC., San Francisco, CA (US)

(21) Appl. No.: 16/317,896

(22) PCT Filed: Jul. 14, 2017
(86) PCT No.: PCT/US2017/042137

§ 371 Date: Jan. 15, 2019

Related U.S. Application Data
(60) Provisional application No. 62/363,161, filed on Jul. 15, 2016.
(60) Provisional application No. 62/404,662, filed on Oct. 5, 2016.
(60) Provisional application No. 62/487,946, filed on Apr. 20, 2017.

(51) Int. Cl. A61B 3/14; A61B 3/12; A61B 3/117; A61B 3/028; A61B 3/16; G06T 7/00; G16H 10/60; G16H 15/00; G16H 30/40

(52) U.S. Cl. CPC A61B 3/145; A61B 3/12; A61B 3/117; A61B 3/028; A61B 3/16; G06T 2207/10016; G16H 10/60; G16H 15/00; G16H 30/40; G06T 2207/30041; G06T 2207/30168; G06T 7/0012

(57) ABSTRACT

Computer readable code adapted and configured to enable collection, annotation and sharing of digital ophthalmic images collected using a camera module on a mobile device or a hand held computer. The hand held computer can be used with a lens adapter configured to engage with a hand held computer device to allow a camera on the hand held computer device to take a high quality image of an eye.

### CROSS REFERENCE TO RELATED APPLICATIONS

This application claims the benefit of U.S. Provisional Patent Application Nos. 62/363,161, filed Jul. 15, 2016, titled “SYSTEMS AND METHODS FOR CAPTURING, ANNOTATING AND SHARING OPHTHALMIC IMAGES OBTAINED USING A HAND HELD COMPUTER”; 62/404,662, filed Oct. 5, 2016, titled “SYSTEMS AND METHODS FOR CAPTURING, ANNOTATING AND SHARING OPHTHALMIC IMAGES OBTAINED USING A HAND HELD COMPUTER”; and 62/487,946, filed Apr. 20, 2017, titled “SYSTEMS AND METHODS FOR CAPTURING, ANNOTATING AND SHARING OPHTHALMIC IMAGES OBTAINED USING A HAND HELD COMPUTER”, which are each herein incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present application relates generally to the field of ophthalmology and systems and methods for improving the treatment and diagnosis of eye conditions in patients in need thereof.

### BACKGROUND

It can be challenging for a patient to get quick and efficient care for eye related problems. Non-ophthalmologists (optometrists, primary care doctors, urgent care doctors, and emergency room doctors) typically will refer a patient to an ophthalmologist for diagnosing any eye related problems. For example, if a patient with an acute problem in the eye or in need of diagnosing other problems with the eye goes to a primary care physician or emergency room they typically are referred to an ophthalmologist for treatment. The non-ophthalmologist may not have the expertise to treat problems associated with the eye or may not be comfortable treating and diagnosing problems associated with the eye based on malpractice concerns or other concerns. Depending on the situation the appointment with the ophthalmologist may not be possible for several days, hours, or weeks. In some cases the closest ophthalmologist may be a long distance from the patient or referring doctor. In emergency situations the costs associated with sending the patient a long distance to an ophthalmologist can be high.

Improved methods for providing information to a patient in need of an eye examination or acute care of the eye are also desired. For example, the present application discloses processes that allow a non-ophthalmologist to obtain patient data from the patient that is relevant to the eye of the patient. The data can be sent electronically to an ophthalmologist for triage and, if necessary, scheduling an appointment with an ophthalmologist based on the severity any condition associated with the patient.

It is also desirable to improve the efficiency of data entry, report generation, and usability of the system for the physicians and healthcare providers using the system.

It is also desirable to improve the efficiency and management of patient records, such as electronic health records (EHR) and electronic medical records (EMR).

### SUMMARY OF THE DISCLOSURE

The present invention relates generally to methods and systems for obtaining, analyzing, and managing patient data relating to the eye of the patient.

In general, in one embodiment, a method for obtaining an image of a retina of a patient, is provided. The method includes: analyzing an image obtained by a camera of a mobile device to look for a contour of an indirect lens along an optical axis of the camera of the mobile device, upon detection of the contour of the indirect lens, determining whether an image of the retina is present in the indirect lens, analyzing the image of the retina to determine one or more predetermined quality parameters associated with the image of the retina obtained by the camera of the mobile device, and providing an indication to a user of the mobile device that corresponds to the one or more predetermined quality parameters associated with the image of the retina obtained by the camera of the mobile device.

This and other embodiments can include one or more of the following features. The method can further include: saving the image of the retina if a predetermined quality threshold is met by the one or more quality parameters associated with the image of the retina obtained by the camera of the mobile device. The method can further include: applying a mask to an area of the image outside of the contour of the indirect lens to create a masked image of the retina. The method can further include: displaying the masked image of the retina on a display of the mobile device. The method can further include: analyzing a plurality of images of the retina and saving a plurality of images of the retina that meet a predetermined quality threshold. The method can further include: saving the plurality of images of the retina that meet the predetermined quality threshold. The plurality of images of the retina can be obtained from a video feed. The plurality of images of the retina can be obtained from a multiple pictures taken by the camera of the mobile device. The plurality of images of the retina that meet the predetermined quality threshold can include a predetermined number of images of the retina. The predetermined number of images can be 10 or less images of the retina. The predetermined number of images can be set by a user of the mobile imaging device. The one or more predetermined quality parameters associated with the image of the retina can include one or more of: glare, exposure, a comparison with an ideal retina image, focus, and lighting. The lens contour can be a substantially circular shape. The method can further include displaying an inverted image of the retina from the indirect lens on a display of the mobile device.

In general, in one embodiment, a method of displaying an image of a retina on a mobile device is provided. The methods can include receiving an image obtained by a camera of a mobile device of an indirect lens along an optical axis of the camera of the mobile device, the image of the indirect lens including an image of a retina of a patient, inverting the image of the indirect lens to form an inverted image of the indirect lens and the retina, and displaying the inverted image of the indirect lens and retina on a display of the mobile device.

This and other embodiments can include one or more of the following features. The indirect lens can have a size of about 10 D to 90 D. The indirect lens can be selected from the group consisting of: 14 D, 20 D, 22 D, 28 D, 30 D, 40 D, or 54 D, 60, 66, and 90 D. The indirect lens can be removably engaged with a lens mount of a lens adapter. The lens adapter can be removably engaged with the mobile device. The lens adapter can include a telescoping arm engaged with the lens mount and a base of the lens adapter engaged with the mobile device. The methods can further include: varying an intensity of a variable intensity light source of the lens adapter engaged with the mobile device to illuminate the retina. Any of the steps can be performed by a mobile application on the mobile device. The mobile device can be a hand held computer device, smartphone, tablet computer, or mobile imaging device. The methods can further include automatically centering the image of the retina on a display of the mobile device. The methods can further include automatically focusing the camera of the mobile device on the image of the retina. The methods can further include presenting the images of the retina that meet a predetermined quality threshold on a display of the mobile device. The methods can further include sending one or more of the images of the retina that meet a predetermined quality threshold to an electronic medical record (EMR) or electronic health record (EHR) of the patient. The methods can further include: automatically saving the one or more images of the retina to the EMR or EHR of the patient. The methods can further include: analyzing a plurality of images of the retina, applying one or more digital image processing techniques to the plurality of the images of the retina, and forming a combined image of the retina based on the plurality of images of the retina and the applied one or more digital image processing techniques.

In general, in one embodiment, a method for obtaining an image of an eye of a patient is provided. The method can include receiving an image of an anterior segment of an eye of a patient with a camera of a mobile device through a lens of a lens adapter engaged with the mobile device, analyzing the image of the anterior segment of the eye to determine one or more quality parameters associated with the image of the anterior segment of the eye, and providing an indication to a user of the mobile device that corresponds to the one or more quality parameters associated with the image of the anterior segment obtained by the camera of the mobile device.

This and other embodiments can include one or more of the following features. The method can further include: saving the image of the anterior segment if a predetermined quality threshold is met by the one or more quality parameters associated with the image of the anterior segment obtained by the camera of the mobile device. The method can further include: varying an intensity of a variable intensity light source of the lens adapter engaged with the mobile device to illuminate the anterior segment of the eye. Any of the steps can be performed by a mobile application on the mobile device. The mobile device can be a hand held computer device, smartphone, tablet computer, or mobile imaging device. The lens can be a macro lens. The lens adapter can include: a body, a clamp configured to engage with the mobile device at a first location and a second location, a lens holder engaged with a macro lens movable between a first position in the optical axis of the camera of the mobile device and a second position outside of the optical axis of the camera of the mobile device, an adjustable light source with a light axis parallel to a macro lens optical axis, a third engagement surface configured to slidably engage with the mobile device at a third location. The clamp can define an axis and the body of the anterior adapter portion is configured to move along the axis of the clamp. The lens adapter can further include: a complementary surface of the body configured to reversibly engage with a base section of a posterior portion. The posterior portion can include the base section configured to reversibly engage with the complementary surface of the body of the lens adapter, a telescoping section movable relative to the base section, and a lens holder engaged with a distal end of the telescoping section configured to removably engage with an indirect lens, the base section configured to removably engage with the body of the anterior adapter portion to form an optical axis between the ophthalmoscopy lens and the camera of the mobile device. The methods can further include automatically focusing the camera of the mobile device on the image of the anterior segment of the eye. The methods can further include presenting the image of the anterior segment of the eye that meet the predetermined quality threshold on a display of the mobile device. The methods can further include sending one or more of the images of the anterior segment of the eye that meet the predetermined quality threshold to an electronic medical record (EMR) or electronic health record (EHR) of the patient. The methods can further include automatically saving the one or more of the images of the anterior segment of the eye to the EMR or EHR of the patient. The methods can include saving the image to a cloud storage network in a HIPAA compliant manner. The image can be encrypted. The non-ophthalmologist can be a primary care doctor, an emergency room doctor, an optometrists, or an urgent care doctor. The methods can further include receiving a plurality of images of the anterior segment of the eye of a patient with the camera of the mobile device through the lens of the lens adapter engaged with the mobile device. The methods can further include analyzing the plurality of images of the anterior segment of the eye of the patient, applying one or more digital image processing techniques to the plurality of the images of the anterior segment of the eye of the patient, and forming a combined image of the anterior segment based on the plurality of images of the anterior segment of the eye of the patient and the applied one or more digital image processing techniques.

In general, in one embodiment, a method is provided. The method includes: receiving images of a portion of an eye of a patient obtained by a non-ophthalmologist with a camera of a mobile device engaged with a lens adapter through a mobile application; sending the images of the portion of the eye of the patient to an ophthalmologist through the mobile application; and receiving notes on the image of the portion of the eye of the patient from the ophthalmologist through the mobile application.

This and other embodiments can include one or more of the following features. The non-ophthalmologist can be a primary care doctor, an emergency room doctor, an optometrists, or an urgent care doctor. The ophthalmologist can be in a referring network with the non-ophthalmologist. The ophthalmologist can be in a referring network of a mobile application database. The methods can further include receiving a referral recommendation from the ophthalmologist for an emergency appointment with an ophthalmologist through the mobile application. The methods can further include receiving a referral recommendation from the ophthalmologist for a non-emergency appointment with an ophthalmologist through the mobile application. The methods can further include receiving an ophthalmology assessment from the ophthalmologist through the mobile application including one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, other eye examination data associated with the patient, and one or more comments from the ophthalmologist. The methods can further include automatically generating a report including the ophthalmology assessment from the ophthalmologist. The methods can further include automatically generating a reimbursement form for the ophthalmologist with billing codes based on the ophthalmology assessment. The image of the portion of the eye of the patient can include an image of a retina of the patient obtained with an indirect lens engaged with the lens adapter. The image of the retina can be obtained using any of the methods described herein. The image of the portion of the eye of the patient can include an image of an anterior segment of the eye of the patient obtained with a macro lens of the lens adapter. The image of the anterior segment can be obtained using any of the methods described herein.

In general, in one embodiment, a method is provided. The method includes: presenting a non-ophthalmologist with a patient in need of an eye examination or acute care of the eye, conducting an examination of the patient by the non-ophthalmologist using a mobile device and a lens adapter removably engaged with the mobile device and a mobile application to generate a patient examination data within the mobile application, sending the patient examination data to an ophthalmologist for review, receiving a patient assessment from the ophthalmologist based on the patient examination data, and sending the patient assessment to the non-ophthalmologist.

This and other embodiments can include one or more of the following features. The non-ophthalmologist can be a primary care doctor, an emergency room doctor, an optometrists, or an urgent care doctor. The ophthalmologist can be in a referring network with the non-ophthalmologist. The ophthalmologist can be in a referring network of a mobile application database. The methods can further include receiving a referral recommendation from the ophthalmologist for an emergency appointment with an ophthalmologist through the mobile application. The methods can further include receiving a referral recommendation from the ophthalmologist for a non-emergency appointment with an ophthalmologist through the mobile application. The methods can further include receiving through the mobile application an assessment from the emergency appointment with the ophthalmologist or an assessment from the non-emergency appointment with the ophthalmologist. The methods can further include sending a notification to the mobile application after the patient sees the ophthalmologist for the emergency appointment or non-emergency appointment. The patient examination data can includes one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, and other eye examination data associated with the patient. the patient assessment from the ophthalmologist includes one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, other eye examination data associated with the patient, and one or more comments from the ophthalmologist. The methods can further include automatically generating a report including the patient assessment from the ophthalmologist. The methods can further include automatically generating a reimbursement form for the ophthalmologist with billing codes based on the patient assessment. The methods can further include automatically populating an electronic health record (EHR) of the patient with the patient examination data and the patient assessment. The image of the portion of the eye of the patient can include an image of a retina of the patient obtained with an indirect lens engaged with the lens adapter. The image of the retina can be obtained using any of the methods described herein. The image of the portion of the eye of the patient can include an image of an anterior segment of the eye of the patient obtained with a macro lens of the lens adapter. The image of the anterior segment can b obtained using any of the methods described herein.

In general, in one embodiment, a method is provided. The method includes: creating an order for an eye examination of a patient, sending the order for the eye examination of the patient to a mobile application, matching a patient ID of the patient to an electronic health record (EHR) for the patient, receiving a patient data point from a non-ophthalmologist using the mobile application and a lens adapter engaged with a mobile device running the mobile application, sending the patient data point to the electronic health record, and automatically populating the electronic health record with the patient data point.

This and other embodiments can include one or more of the following features. The non-ophthalmologist can be a primary care doctor, an emergency room doctor, an optometrists, or an urgent care doctor. The methods can include sending instructions for the eye examination of the patient through the mobile device to the non-ophthalmologist. The patient examination data can include one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, and other eye examination data associated with the patient. The image of the portion of the eye of the patient can include an image of a retina of the patient obtained with an indirect lens engaged with the lens adapter. The image of the retina can be obtained using any of the methods described herein. The image of the portion of the eye of the patient includes an image of an anterior segment of the eye of the patient obtained with a macro lens of the lens adapter. The anterior segment can be obtained using any of the methods described herein.

In general, in one embodiment, a method is provided herein. The method is includes: receiving a patient data point including eye examination data collected with a mobile application with a lens adapter engaged with a mobile device running the mobile application, receiving an assessment of the patient data point done by an ophthalmologist with the mobile application, receiving an electronic signature from the ophthalmologist; automatically generating billing codes that correspond to the patient data point and the assessment of the patient data point, automatically generating a report including the billing codes, patient data point, and the assessment of the patient data point, and submitting the report for reimbursement.

This and other embodiments can include one or more of the following features. The patient data point can be collected by a non-ophthalmologist. The non-ophthalmologist can be a primary care doctor, an emergency room doctor, an optometrists, or an urgent care doctor. The patient examination data can includes one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, and other eye examination data associated with the patient. The assessment of the patient data point done by the ophthalmologist can include one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, other eye examination data associated with the patient, and one or more comments from the ophthalmologist. The image of the portion of the eye of the patient can include an image of a retina of the patient obtained with an indirect lens engaged with the lens adapter. The image of the retina can be obtained using any of the methods described herein. The image of the portion of the eye of the patient can include an image of an anterior segment of the eye of the patient obtained with a macro lens of the lens adapter. The image of the anterior segment can be obtained using any of the methods described herein.

In general, in one embodiment, a system is provided. The system includes: a mobile imaging device with a camera. The mobile imaging device can be configured to run a computer executable code comprising any of the steps described herein. The system can include a lens adapter configured to removably engage with the mobile imaging device. The system can include an adapter configured to engage with a hand held computer device with a camera having an optical axis comprising: an anterior adapter portion comprising: a body, a clamp configured to engage with the hand held computer device at a first location and a second location, a lens holder engaged with a macro lens movable between a first position in the optical axis of the camera and a second position outside of the optical axis of the camera, an adjustable light source with a light axis parallel to a macro lens optical axis, a third engagement surface configured to slidably engage with the hand held computer device at a third location, and a complementary surface of the body configured to reversibly engage with a base section of a posterior portion, wherein the clamp defines an axis and the body of the anterior adapter portion is configured to move along the axis of the clamp; and the posterior portion comprising: the base section configured to reversibly engage with the complementary surface of the body of the anterior adapter portion, a telescoping section movable relative to the base section, and a lens holder engaged with a distal end of the telescoping section configured to removably engage with an ophthalmoscopy lens, the base section configured to removably engage with the body of the anterior adapter portion to form an optical axis between the ophthalmoscopy lens and the camera of the hand held computer device.

This and other embodiments can include one or more of the following features. The system can further include: a removable enclosure configured to removably engage with the posterior portion. The removable enclosure can include a clamping mechanism to engage with the posterior portion. The removable enclosure can further include: a telescoping portion configured to adjust a length of the removable cover. The removable enclosure can further include a proximal portion with an opening to accommodate the camera of the hand held computer device and the light source of the anterior adapter portion and a distal section to engage with the lens holder. The removable enclosure can be adapted to encase the optical pathway between the camera and the lens holder.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a flow chart illustrating a method of obtaining patient data with a hand held computer device in accordance with some embodiments.

FIG. 2 illustrates a flowchart illustrating an electronic method for handling patient data in accordance with some embodiments.

FIG. 3 shows a flowchart of an electronic method for using optical character recognition to identify a patient in accordance with some embodiments.

FIG. 4 is a flow chart illustrating a method of obtaining patient data with a hand held computer device in accordance with some embodiments.

FIG. 5 is a flow chart illustrating a method of obtaining patient data with a hand held computer device in accordance with some embodiments.

FIG. 6 is a flow chart illustrating an electronic method of obtaining patient data in accordance with some embodiments.

FIG. 7 illustrates an exemplary embodiment of an electronic notification and tracking process that can be provided by the application and backend.

FIG. 8 illustrates a sample flowchart for preparing various reports with a hand held computer device in accordance with some embodiments.

FIG. 9 illustrates a sample report from an assessment that can be generated using the systems and methods described herein.

FIGS. 10A-10Q illustrate examples of screen shots of an application with a user interface (UI) on a mobile device in accordance with some embodiments.

FIGS. 11A-11B illustrate examples of screen shots of an application with a user interface (UI) on a mobile device in accordance with some embodiments.

FIGS. 12A-12B illustrate examples of screen shots of an application showing an image of a portion of a retina on a mobile device in accordance with some embodiments.

FIGS. 13A-13C illustrate examples of screen shots of an application with a UI on a mobile device in accordance with some embodiments.

FIG. 14 shows a flow chart of a method in accordance with some embodiments.

FIG. 15 illustrates examples of screen shots of an application with a UI on a mobile device in accordance with some embodiments.

FIG. 16 shows a flow chart of a method in accordance with some embodiments.

FIG. 17 shows a flow chart of a method in accordance with some embodiments.

FIG. 18 shows a flow chart of a method in accordance with some embodiments.

FIG. 19 is a front view of an adapter attached to a hand held computer device in accordance with some embodiments.

FIG. 20 is a front view of an adapter in accordance with some embodiments.

FIG. 21 is a back view of an adapter in accordance with some embodiments.

FIG. 22 is a side view of an adapter in accordance with some embodiments.

FIG. 23 illustrates an anterior portion and posterior portion of an adapter in accordance with some embodiments.

FIG. 24 is a front view of an anterior portion of an adapter attached to a hand held computer device in accordance with some embodiments.

FIG. 25 is a back view of an anterior portion of an adapter in accordance with some embodiments.

FIG. 26 is a front view of an anterior portion of an adapter attached to a hand held computer device in accordance with some embodiments.

FIG. 27 is a side view of an anterior portion of an adapter attached to a hand held computer device in accordance with some embodiments.

FIG. 28 is a front view of an adapter attached to a hand held computer device in accordance with some embodiments.

FIG. 29 is another front view of an adapter attached to a hand held computer device in accordance with some embodiments.

FIG. 30 is a back view of an adapter attached to a hand held computer device in accordance with some embodiments.

FIG. 31 is a side view of an adapter attached to a hand held computer device in accordance with some embodiments.

FIG. 32 is a front view of an adapter attached to a hand held computer device in accordance with some embodiments.

FIGS. 33 and 34 are front views of an anterior portion of an adapter engaged with a hand held computer device with a macro lens in the optical pathway of the camera of the hand held computer device in accordance with some embodiments.

FIGS. 35, 36 and 37 are back, side, and head on views of an anterior portion of an adapter engaged with a hand held computer device with a macro lens in the optical pathway of the camera of the hand held computer device in accordance with some embodiments.

FIG. 38A illustrates a side view of an anterior portion of an adapter engaged with a hand held computer device with a macro lens in the optical pathway of the camera of the hand held computer device in accordance with some embodiments.

FIG. 38B illustrates a side view of an anterior portion and posterior portion of an adapter engaged with a hand held computer device with a macro lens out of the optical pathway of the camera of the hand held computer device in accordance with some embodiments.

FIG. 39 illustrates a side view of an anterior portion and posterior portion of an adapter engaged with a hand held computer device with a macro lens out of the optical pathway of the camera of the hand held computer device in accordance with some embodiments.

FIG. 40 illustrates a side view of an adapter engaged with a hand held computer device and optical pathway enclosure adapter in accordance with some embodiments.

FIG. 41 is an example of a cross-sectional view through the optical pathway enclosure in accordance with some embodiments.

FIGS. 42A and 42B illustrate an optical pathway enclosure adapter engaged with an adapter in accordance with some embodiments.

FIGS. 42C and 42D are cross-sectional and exploded views of an optical pathway enclosure adapter in accordance with some embodiments.

FIGS. 43A and 43B illustrate additional embodiments of an anterior adapter engaged with a hand held computer device in accordance with some embodiments.

FIGS. 43C-43G illustrate additional features of embodiments of anterior adapters described herein.

FIGS. 44A and 44B illustrate an exterior view and cross-sectional view, respectively, of a removable beam splitter module in accordance with some embodiments.

FIGS. 44C and 44D illustrate the beam splitter module separate from and engaged with an anterior adapter, respectively, in accordance with some embodiments.

FIGS. 44E and 44F illustrate a front and back view respectively of a beam splitter module in accordance with some embodiments.

FIG. 45A illustrates an anterior adapter engaged with an embodiment of a beam splitter module in accordance with some embodiments.

FIG. 45B illustrates an anterior adapter engaged with an embodiment of a slit lamp module in accordance with some embodiments.

FIG. 45C illustrates an anterior adapter engaged with an embodiment of a collimated beam module in accordance with some embodiments.

FIG. 45D illustrates an anterior adapter engaged with an embodiment of a mask module in accordance with some embodiments.

FIGS. 46A-46D illustrate embodiments of modules with multiple lenses that can be used with the adapters described herein.

FIG. 47A illustrates an adapter with a posterior portion having an integral telescoping optical pathway enclosure in accordance with some embodiments.

FIG. 47B illustrates an adapter with a posterior portion having an integral telescoping optical pathway enclosure in accordance with some embodiments.

FIGS. 48A-48D illustrate various views of an anterior adapter portion in accordance with some embodiments including a front-view, cross-sectional view, back view and front view, respectively.

FIGS. 49A and 49B illustrate a front and back view of an anterior adapter portion in accordance with some embodiments.

FIGS. 50A and 50B illustrate a front and back view, respectively of an adapter engaged with a hand held computer device in accordance with some embodiments.

FIGS. 51A-51C illustrate various views of an anterior adapter portion engaged with a hand held computer device with the posterior portion separate from the anterior portion in accordance with some embodiments.

### DETAILED DESCRIPTION

The present application discloses systems and methods for obtaining a patient data point and can send the patient data to an experienced physician for an assessment. The patient data point is collected electronically through a mobile imaging device like a hand held computer device by a healthcare provider. The present application focuses on the workflow for providing eye care to a patient; however, other applications can also be used, such as dermatology and other health care practice areas. The methods can include computer assisted methods, electronic methods, or using an application with computer readable code operable on a mobile device with a camera such as a hand held computer device, smartphone, tablet computer, or mobile imaging device. Also disclosed herein are mobile applications that can be used with the mobile device, hand held computer device, smartphone, tablet computer, or mobile imaging device to streamline collecting patient data, providing feedback on the patient, etc.

The patient data point can be collected using the camera on the mobile device, an optional adapter for the mobile device like a lens adapter, and/or sensors on board the mobile device and electronically entered into the application. In other cases a conventional eye examination device can wirelessly send data to the mobile device. In some cases data or information collected by the conventional eye examination device can be manually input into the application on the mobile device by the physician or through optical character recognition. In some cases images can be obtained using other imaging devices besides a mobile device and input into the applications described herein. For example a conventional examination of the anterior segment or fundus can be done using conventional commercial imaging devices and sent to the applications described herein. The images can be received by the application from the conventional commercial imaging devices through wired or wireless data transfer or other data or image transmission techniques.

In some aspects, a patient data point refers to a representation in electronic form of one or more indicia of the ophthalmic health of the patient. Electronic form of the patient data may exist in a number of different formats depending upon the specific clinical requirements of the information and how it will be used to provide health outcome for the patient or to guide an episode of care between a health care provider, a patient, and one or more ophthalmic specialists. The representation in electronic form of the one or more indicia of the ophthalmic health of the patient can be added to or integrated with an electronic healthcare record or electronic medical record as described herein.

The patient data point is collected by a healthcare provider or physician using the application. The application can be designed for use by a physician or healthcare provider and not for use by patients. The healthcare provider can be a nurse, physician, or other healthcare provider. The healthcare provider can typically be a physician who is not an ophthalmologist, e.g., a non-ophthalmologist. Non-limiting examples of non-ophthalmologists include: a primary care doctor, an emergency room doctor, a retina specialist, an optometrist, or an urgent care doctor.

In still other aspects, embodiments of a computer readable code executable on a mobile device are disclosed herein. For example, the computer readable code can include an application on a smartphone, iPod, mobile device, imaging device, tablet computer, or other hand held device that includes a processor and display. Also described herein are an application and a user interface (UI) that can include computer readable instructions available locally or via a remote server, distributed server or a cloud resource. A number of different capabilities, features and configurations are possible separately or as a collection of features that may be used on a hand held computer device, such as a mobile device.

In one example for providing eye care to a patient, the users of the application and system can include resident doctors, emergency room doctors, attending ophthalmologist, retina specialists, optometrists, etc. The resident doctor typically has moderate eye care experience and has regular involvement in eye imaging. The resident doctor typically examines a high volume of patients and will typically refer complex eye cases to a specialist like an ophthalmologist. An emergency doctor (ED)/physician typically has low eye care experience and is rarely involved with eye imaging. The ED typically examines a high volume of patients and refers patients for complex eye cases to a specialist like an ophthalmologist. A retina specialist typically has high eye care experience but typically does not have involvement with imagining eyes. An optometrist typically has moderate eye care experience and occasional involvement in eye imaging. The optometrist typically sees a low volume of patients and usually refers patients in complex eye care cases to a specialist like an ophthalmologist. An attending ophthalmologist typically has high eye care experience. The ophthalmologist typically does not have involvement with imaging the eyes and usually treats a medium volume of patients. For complex eye cases the attending ophthalmologist treats the patients and makes assessments. The resident doctor, ED, retina specialist, and optometrist would typically refer complex cases to the attending ophthalmologist or other ophthalmologist.

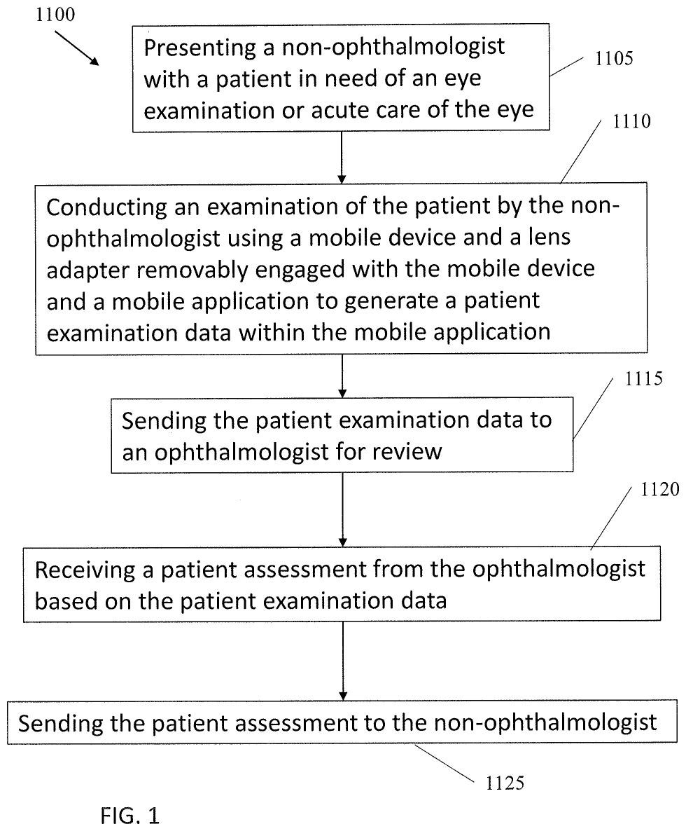

FIG. 1 is a flow chart illustrating a method 1100 of obtaining patient data with a hand held computer/mobile device in accordance with some embodiments. The methods can include presenting a non-ophthalmologist with a patient in need of an eye examination or acute care of the eye 1105. Examples of the non-ophthalmologist include the resident doctor, ED, primary care provider, retina specialist, optometrist, etc. The methods can include conducting an examination of the patient by the non-ophthalmologist using a mobile device and a lens adapter removably engaged with the mobile device and a mobile application to generate a patient examination data within the mobile application 1110. The methods can include sending the patient examination data to an ophthalmologist for review 1115. The patient examination data can be reviewed by the ophthalmologist. The ophthalmologist can review the image and provide notes, assessment, or comments on the patient examination data and optionally a referral to an ophthalmologist for further care. In some cases the ophthalmologist's input is used by the application to generate a SOAP note (Subjective, Objective, Assessment and Plan). The assessment/referral/comments on the patient examination data can then be sent to the non-ophthalmologist. The methods can include receiving a patient assessment from the ophthalmologist based on the patient examination data 1120. The methods can include sending the patient assessment to the non-ophthalmologist 1125.

In some embodiments the ophthalmologist is in a referring network with the non-ophthalmologist. In some embodiments the ophthalmologist is in a referring network of a mobile application database. The methods can further include receiving a referral recommendation from the ophthalmologist for an emergency appointment with an ophthalmologist through the mobile application. The methods can further include receiving a referral recommendation from the ophthalmologist for a non-emergency appointment with an ophthalmologist through the mobile application. The methods can further include receiving through the mobile application an assessment from the emergency appointment with the ophthalmologist or an assessment from the non-emergency appointment with the ophthalmologist. The methods can further include sending a notification to the mobile application after the patient sees the ophthalmologist for the emergency appointment or non-emergency appointment.

In some embodiments the patient examination data includes one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, and other eye examination data associated with the patient. In some embodiments the patient assessment from the ophthalmologist includes one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, other eye examination data associated with the patient, and one or more comments from the ophthalmologist. The methods can further include automatically generating a report including the patient assessment from the ophthalmologist. The methods can further include automatically generating a reimbursement form for the ophthalmologist with billing codes based on the patient assessment. The methods can further include automatically populating an electronic health record (EHR) of the patient with the patient examination data and the patient assessment.

The image of the portion of the eye of the patient can include an image of a retina of the patient obtained with an indirect lens engaged with the lens adapter. The image of the retina can be obtained using any of the methods described herein. The image of the portion of the eye of the patient can include an image of an anterior segment of the eye of the patient obtained with a macro lens of the lens adapter. The image of the anterior segment can be obtained using any of the methods described herein.

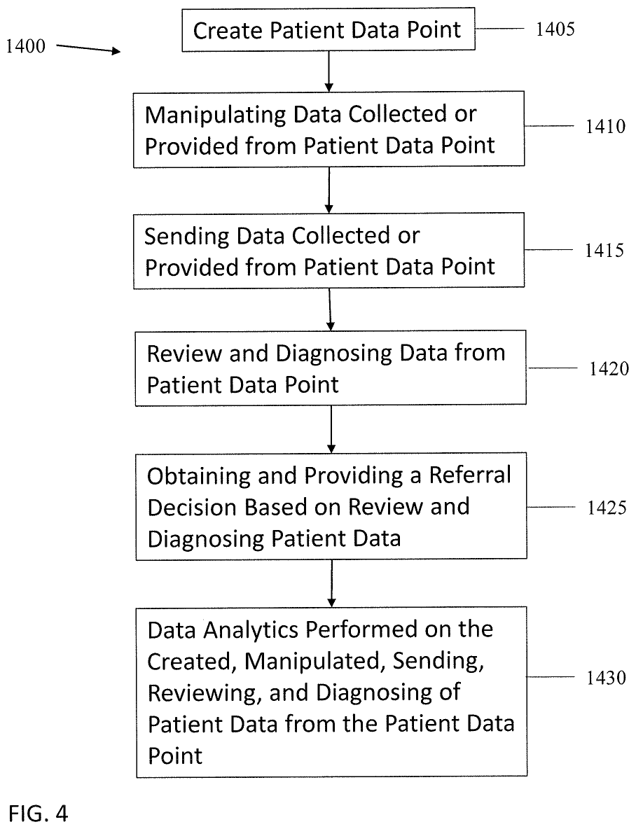

FIG. 4 is a flow chart illustrating a method 1400 of obtaining patient data in accordance with some embodiments. The method includes creating a patient data point 1405. The patient data point can include any of the patient data described herein. Next, the method can include manipulating data collected or provided from the patient data point 1410. Manipulating the patient data can include modifying the image that is acquired. Manipulating the patient data can include drawing images on the image and annotating to indicate an area of interest on the image. A time stamp or other identifier can be added to the patient images. The method can further include sending data collected or provided from the patient data point 1415. Sending the data can be accomplished in a compliant manner. The patient data can be sent to an individual, such as another physician, an ophthalmologist for a referral, etc. The data can also be sent to a group of people, such as a group of ophthalmologists for a referral request. Data can be stored in the cloud and accessed via a link within the application. In some cases the links can be sent via text or e-mail. Next, the method can include reviewing and diagnosing data from the patient data point 1420. The review can be done by an ophthalmologist. The patient data point can be sent to a list of ophthalmologists on the user's referral list. In some cases where speed is important, the patient data point can be sent to a large number, like all or many ophthalmologists using the application, for analysis and assessment. The method can include obtaining and providing a referral decision or assessment based on the review and diagnosing of the patient data 1425. The method can also include data analytics performed on the created, manipulated, sending, reviewing, and diagnosing of the patient data from the patient data point 1430. The data analytics can provide information on all of the referral requests sent by the user along with data about referral response time, referral results, etc. The analytics data can help the user learn more about the efficiency for different referral ophthalmologists and by curating the referring doctor list can improve the response time and rate for future referral requests. The analytics can also be used to aggregate and analyze the overall workflow to better understand the clinical practices and to improve the overall workflow.

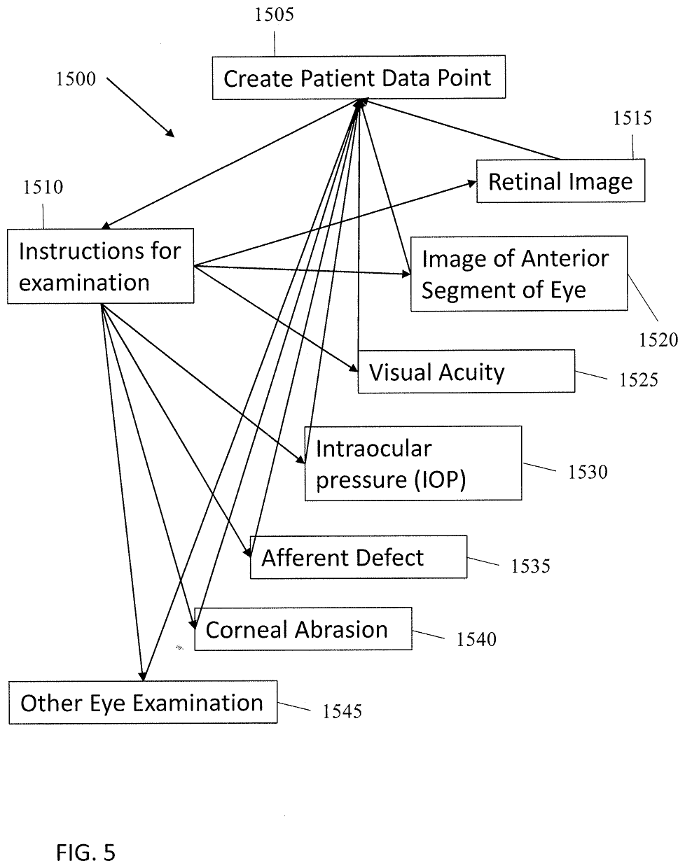

FIG. 5 is a flow chart illustrating a method 1500 of obtaining patient data in accordance with some embodiments. Creating the patient data point 1505 can be done using a variety of different techniques, examinations, and different medical devices. The patient data point can be collected by a non-ophthalmologist. The patient data point can be manually entered into a smartphone application or can be collected using the smartphone application. Examples of patient data points include: retinal image 1515, image of an anterior segment of the eye 1520, visual acuity (VA) 1525, intraocular pressure (IOP) 1530, afferent defect 1535, corneal abrasion 1540, and other eye examination results 1545. The smartphone application can provide instructions 1510 for the healthcare provider or non-ophthalmologist to collect the patient data points. For the example, the instructions could walk the non-ophthalmologist through the steps for using a smartphone and a lens adapter system to obtain an image of the anterior segment of the eye.

The patient data point can include a retinal image and/or an image of an anterior segment of the eye. The image of the anterior segment of the eye can be obtained with a camera and a lens, such as a macro lens. The image of the retina/posterior segment of the eye can be obtained using an ophthalmoscopy lens with or without dilating the pupils (e.g., mydriatic or non-mydriatic techniques). Various smartphone adapters for retinal imaging are known, including those disclosed in US 2012/0320340, WO 2014/194182, and co-pending U.S. patent application Ser. No. 15/186,266 entitled “Adapter for Retinal Imaging Using a Hand Held Computer”, which is published as US 2016/0367135. Any of the smartphone adapters disclosed in US 2012/0320340, WO 2014/194182, and co-pending U.S. patent application Ser. No. 15/186,266 published as US 2016/0367135 can be used to obtain a retinal image and/or an image of an anterior segment of the eye. A slit lamp adapter can be used with the mobile device to obtain an image of the patient's eye. An example of a slit lamp adapter that can be used with is a slit lamp module available from the Digital Eye Center.

The patient data point can include visual acuity and contrast test results. The test results can be obtained using conventional methods, such as a Snellen chart. In some cases the test results can be performed using a tablet computer, smartphone, or mobile device. Examples of vision tests that can be performed using a tablet computer, smartphone, or mobile device include visual acuity, contrast, etc.

The patient data point can include intraocular pressure (IOP). The IOP can be obtained using a conventional tonometer. The IOP can be manually input into the application or can be electronically input if a smart tonometer is used to measure the IOP.

The patient data point can include information relating to an afferent defect (relative afferent pupillary defect (RAPD)). The RAPD can be obtained using conventional methods such as by swinging a bright light. A flashlight or smartphone device having a flash can be used to obtain information relating to the RAPD.

The patient data point can include information relating to corneal abrasion. Conventional methods can be used to determine a corneal abrasion, such as by putting a fluorescent dye on the eye and observing the eye under a blue light. Areas of the eye with corneal abrasions will pick up the dye differently than non-injured portions of the eye and will look different than non-injured portions of the eye when illuminated with blue light. The eye can be visually observed to determine the presence or absence of corneal abrasions. The patient data point can include an image of the eye with the dye under blue light or a note by the physician indicating the absence/presence of corneal abrasions and optionally the location of the abrasions.

The patient data point can include images taken using other imaging devices besides a mobile device. For example a conventional examination of the anterior segment or fundus can be done using conventional commercial imaging devices and input into the applications described herein.

The patient data point can include other types of vision related tests. In one example, the patient data point can include a metamorphopsia test, such as results obtained using an Amsler grid. Examples of metamorphopsia tests that can be performed using a tablet computer, smartphone, or mobile device include tests using Amsler grids on the display of the tablet computer, smartphone, or mobile device. Another example of a patient data point that can be obtained using other types of vision related tests includes visual field. Other examples of patient data points include: color blindness test, cover test, ocular motility testing (from smartphone video), tear film break (from smartphone video or calculated by the application), stereopsis (depth perception) test, retinoscopy, refraction, autorefraction, slit lamp examination, and pupil dilation.

Other examples of patient data points that can be input into the application include: 1. hertel measurement (exophthalmometer), 2. visual field testing (short-wave automated like blue on yellow perimetry, kinetic, and static), 3. IOP, 4. slit beam, 5. stereo photography, 6. fluorescence (cobalt blue filter/anterior segment), 7. hyperacuity, 8. color vision (red/green, yellow/blue, Farnsworth 15/100), 9. contrast sensitivity, 10. refractive error, 11. potential acuity, 12. pupils (afferent defect, size, reactivity, accommodation), 13. non-mydriatic fundus photography, 14. eyelid position, 15. extra ocular motility, 16. strabismus (quantitative) for tropia/phoria/cyclodeviation (double Maddox rod), 17. corneal topography, 18. retinal thickness/microstructure/optical coherence tomography (OCT), 19. ultrasound or equivalent cross-sectional imaging, 20. anterior segment, 21. posterior segment, 22. biometry (axial length), and 23. gonioscopy. The mobile device can run computer readable instructions used in the application operating on the mobile device. The compute readable instructions can be modified to include steps that are useful or necessary for collecting patient data points for any of the eye care related test described herein. The computer readable instructions used in the application running on the mobile device can include capturing, processing, sharing, annotating, or providing information related to one or more of these 23 different characters this characteristics of an ophthalmic examination or treatment of the eye

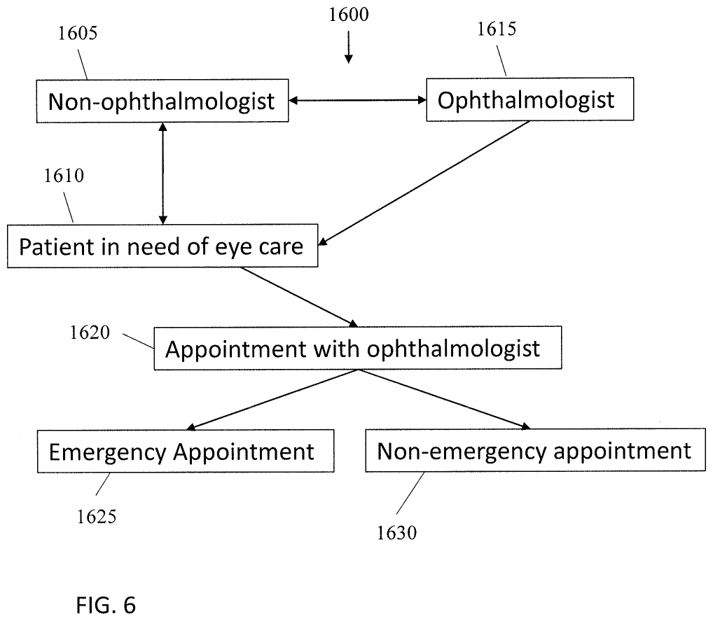

FIG. 6 is a flow chart illustrating an electronic method 1600 of obtaining patient data in accordance with some embodiments. A patient in need of eye care 1610 visits a non-ophthalmologist for treatment. The non-ophthalmologist 1605 can collect patient data during the examination. The patient data point can be sent to an ophthalmologist 1615 for triage or feedback. After the ophthalmologist reviews the patient data point the comments or referral based on the patient data point from the ophthalmologist can be sent to the non-ophthalmologist. The patient can review the comments or referral from the ophthalmologist with the non-ophthalmologist and decide on next steps for treatment. In one example, the patient can directly schedule a follow up appointment with an ophthalmologist 1620 to receive treatment for a non-emergency situation 1630. If the patient data point indicates a possible emergency situation then the patient can be sent directly to an ophthalmologist for emergency medical care 1625.

In some embodiments the ophthalmologist can forward the patient information and request for an assessment to another subset of ophthalmologists/eye care specialists for comments, information, or an assessment. One or more of the ophthalmologists/eye care specialists receiving the request can provide comments or an assessment to the ophthalmologist. The ophthalmologist can then review the comments from the ophthalmologists/eye care specialists and add additional notes and/or provide an updated assessment based on this information to the non-ophthalmologist. The data analytics can analyze the overall referral chain to calculate reimbursement for the physicians providing information used in the assessment.

The application can allow messaging between other users of the application. The messaging, push notifications, image sharing, patient data record sharing, and other transmissions of patient data can be done in a HIPAA compliant manner. The application and back end (including cloud and remote networks) can perform any of the data workflows shown in the flowcharts illustrated in the figures.

The images taken by the mobile device can be stored on the mobile device in an encrypted format. The encryption and storage can prevent an unauthorized user from accessing the images. Image sharing can be done by uploading the images to the cloud or back end followed by sharing a link within the application to the other user, such as the referral target. The referral target can click on the link to load the image from the cloud in temporary memory on the mobile device.

The application and backend of the software can improve the integration with electronic health records (EHR). For example, the patient data points can be automatically sent to and included in the patient's electronic medical record (EMR) or electronic health record (EHR). Different hospitals, private practices, doctors, and healthcare providers can use different programs and processes for managing electronic health records. Integration with the legacy programs and processes that are used by the healthcare provider is important. The users of the legacy programs and processes do not want to have to use a separate portal or system to access data. It is desirable for all of the medical records and information to appear in a single system. In some embodiments the patient data points collected as described herein can be automatically added to the EHR.

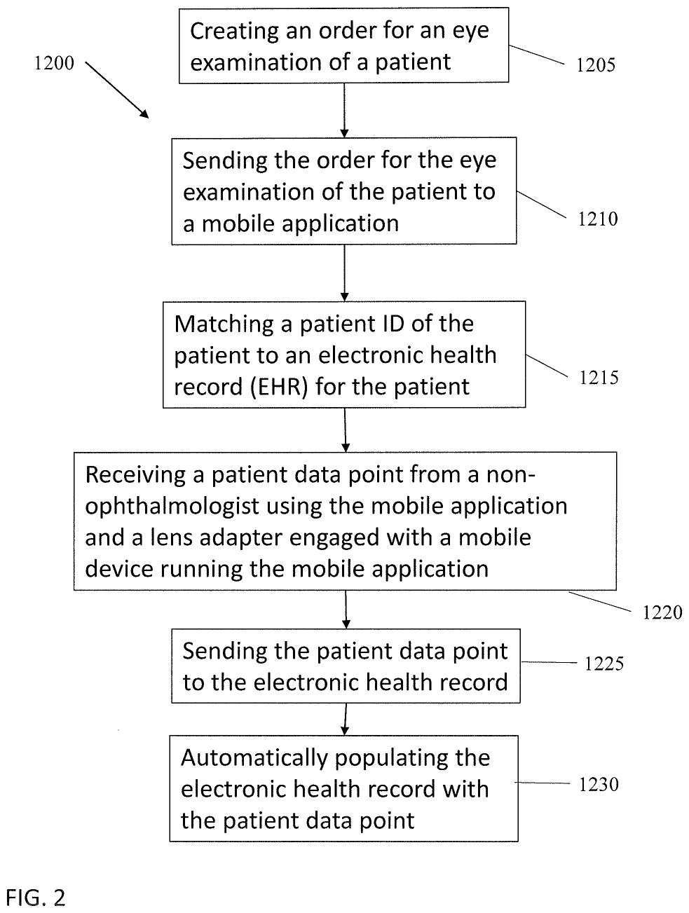

FIG. 2 shows a flowchart of a method 1200 in accordance with some embodiments. The method can include creating an order for an eye examination of a patient 1205. In a first step the healthcare provider creates an order for an eye examination of the patient. The order can be created in the computer system used by the healthcare provider. The method can include sending the order for the eye examination of the patient to a mobile application 1210. In one example, the healthcare provider can use the EPIC healthcare management system. The order in EPIC is sent to the eye care platform, such as the mobile application and software described herein. One challenge with interoperability between multiple electronic record systems is matching and confirming the patient IDs. The method can include matching a patient ID of the patient to an electronic health record (EHR) for the patient 1215. The eye care platform matches the patient ID from the electronic health record with the patient ID in the eye care platform. After the order is entered into the eye care platform the patient data point corresponding to the order is collected through the eye care platform application. The method can include receiving a patient data point from a non-ophthalmologist using the mobile application and a lens adapter engaged with a mobile device running the mobile application 1220. The method can include sending the patient data point to the electronic health record 1225. The patient data point is then sent to the electronic health record, such as the patient record in EPIC. The method can include automatically populating the electronic health record with the patient data point 1230. The electronic health record can then be automatically populated with the patient data point. The patient data point can include text and images that are added to the EHR. The handling of the patient data points and EHR can be accomplished using cloud data service hosting, quality/security, and EHR integration. The patient data points can comply with fast healthcare interoperability resources (FHIR). The methods can further include sending instructions for the eye examination of the patient through the mobile device to the non-ophthalmologist. The patient examination data can include one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, and other eye examination data associated with the patient. The image of the portion of the eye of the patient can include an image of a retina of the patient obtained with an indirect lens engaged with the lens adapter. The image of the retina can be obtained using any of the methods described herein. The image of the portion of the eye of the patient can include an image of an anterior segment of the eye of the patient obtained with a macro lens of the lens adapter. The image of the anterior segment can be obtained using any of the methods described herein.

The application can present a user interface (UI) to the physician via the display on the mobile device. The user interface can include a chronological listing or feed similar to a social media timeline that includes patient medical records. The feed can be based on forward/reverse chronological order, priority, or other predefined characteristics. The healthcare provider can add to the medical records directly through the application. The chronological listing can include a list of encounters by the patient with each encounter including all of the notes, patient data points, and information pertaining to an appointment with a healthcare provider. The timeline can allow the healthcare provider to quickly access past information relating to the patient to quickly access patient health information, referral notes, assessments by ophthalmologists, etc.

The UI can include a workflow for the physician and healthcare provider to manage the patients in the office and patients with appointments scheduled that day. The workflow can be based on chronological order, priority, or other predefined characteristics or combination of characteristics for the patients that are in the office for appointments or have appointments scheduled for that day. The physician or healthcare provider can use the timeline to manage the workflow for the patients. The workflow can include a list of “Encounters” (e.g. patient examinations) that are in progress, finished, or flagged for follow up. The order of the patients can be displayed in chronological order. The order of the encounters can be modified by the user of the application. For example, the order of encounters can be dragged and dropped to change the order of patients to manage the office workflow to quickly re-prioritize different patients. A touchscreen on the mobile device can be used to modify the order.

The application can allow the healthcare provider to keep encounters selected at the top for follow up. The UI can include a left indicator that can show the need for follow up. The healthcare provider or physician can sort to show open items, create or review a to do list, show a status list, show a priority list, etc.

The application and UI can allow the physician or healthcare provider to select a patient and show: chronological results; images/IOP, notes, visual acuity, other test results, along with the name of note taker. The physician and healthcare provider can use the application to search through notes/images. The application and UI can allow the user to take and add a new patient data point, such as a new picture of the posterior or anterior of the eye. The new patient data point may be collected using a smart phone or mobile device operating an embodiment of the application described herein.

The application and UI can allow for collaboration with other users of the application. For example, the application and UI can support a chat feature that allows multiple users of the application to send and receive messages from one another. The application and UI can show if the other person in the chat is online and whether they have a stable internet connection.

The application and UI can include a referral tab. The referral tab can allow the user to input and manage the team of ophthalmologists that are used for referrals. A backend of the application can analyze the referral statistics to show details of all referrals.

The application and UI can include an encounters list. Each patient that has a scheduled appointment can be included on the encounters list. The encounters can correspond to patient appointments and can include patient information, notes about the appointment, patient data collected during the appointment, and other relevant information. Information included in the encounter can include any interaction with the health care provided related to the treatment of the patient's eye. The encounters list can include a list of patient encounters sorted and re-ordered by date of latest data added. The encounters list includes the functionality to add a new patient or select an existing patient and patient search.

The application and UI can include location information for patient encounters. The locations can be used as a “tag” for patient encounters. Patient encounters can be sorted using the location tag. For example, the user can select to view encounters in “all locations”, “location 1”, “location 2”, etc.

The application and UI can include a patient timeline. The patient timeline can include the patient data displayed in a chronological scrollable timeline. Various tests can be added to the timeline. For example, “Add Note” and “Add Photo” buttons can be used to add information to the patient timeline. The patient timeline can also include text fields to input additional patient data points, such as VA or IOP data.

The application and UI can allow for a ping to be sent to a practice member. The practice member list can include all the practice physicians in the particular practice. The practice member list can be used as a pick list when sending “pings” to a practice member. The practice member list can be populated directly in the back end. The ping can be in the form of a push notification, system icon notification, and an in-app dot notification, which appears next to the encounter. The dot goes away after a user with the notification takes any action on the patient timeline, such as add a photo or add a note. Any provider from the practice member list can change the ping to another provider, in which case the new provider gets the dots and the push notification and the previous provider's dot goes away. The ping or notification can be sent for a read receipt when the ophthalmologist reviews the patient data record and when the ophthalmologist provides an assessment of the patient data.

The application and UI can include a camera interface for using the camera onboard the mobile device. The camera interface can be used to add medical photos of the eye to the patient timeline. The application and UI can include a section to add notes to the patient timeline.

The application and UI can include a screen with options for signing in to the application, a logout option, an invert fundus image option, and a location management option that can give the users the ability to add locations.

The application and UI can include an offline option to use the application without an internet, data, or cellular connection. Any data input into the application in offline mode can be uploaded to the cloud/remote computer network when the application later is connected to an internet, data, or cellular connection.

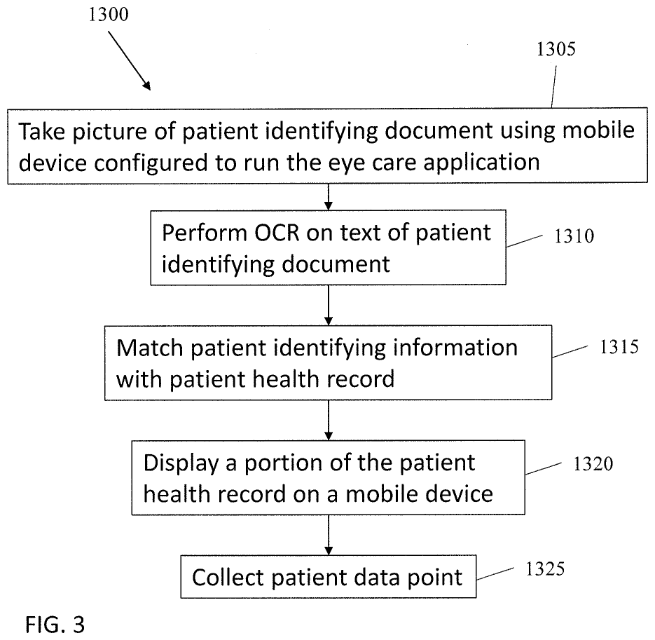

The application and UI can include optical character recognition (OCR), bar code scanning, or other method to input data into the application. FIG. 3 shows a method 1300 for using OCR to identify a patient. A picture is taken of the patient identifying document 1305 and then OCR is performed on the text of the patient identifying document 1310. Next, the patient identifying information is matched to the patient health record 1315. The application can then display a portion of the patient health record on the mobile device 1320. The user of the mobile device can then collect the patient data point 1325. The patient data point can be collected without the need to manually input patient information. In one example a picture can be taken of the driver's license, insurance card, hospital wrist band, passport, or other document associated with the patient with OCR then used to scan the driver's license, insurance card, or other document associated with a patient for relevant information. The ability to take a picture and have the relevant identifying text automatically input into the application can save the physician a lot of time with inputting patient information using manual methods like typing. The application and back end system can analyze the identifying information from the OCR of the image of the identifying document and match that information to a patient record in the application database or an EHR. The OCR features can also be used to take a picture of a device reading to recognize the value or result from the test with the number being automatically imported into the patient timeline.

The application and UI can also be setup to minimize typing and data entry required by the physician using the application. OCR can be used to populate patient data. Voice recognition, gesture shortcuts, and pre-populated position preferences can also be used to improve input of information into the application. The pre-populated references can be provided based on machine learning or an analysis of common features for patients with similar physical characteristics or examination histories. In some cases dictation can be used to avoid typing. Image capture features can be used to auto capture a quality image of the patient anatomy. The application can also automatically suggest and include billing reimbursement codes associated with the collection of the patient data point and the assessment process. For example, under current practices the physician may need to use a separate computer or system to look up the billing codes corresponding to the patient data point, assessment, or other learning during the appointment. The physician then has to manually type the billing code in a separate system as part of the documentation process and reimbursement process. The application can automatically populate reimbursement codes within sections of the application to streamline the preparation of reimbursements documentation. The application can also provide automatic suggestions or a curated drop down menu with suggested billing codes to save physician time looking up codes and manually entering information.

The application and UI can include the option to add other photos to the encounter or patient timeline. In one example a photo of the OCT screen can be taken and added to the patient timeline. In another example a picture of a Tonopen screen (for IOP) can be taken and automatically added to the patient timeline. Other relevant photos can also be added to the patient timeline.

The application and UI can include the ability to favorite or pin favorite encounters within the application. Favoriting or pinning the encounter can allow for quicker access to the encounter for review or sharing.

The application and UI can include the ability to block or prevent screen captures of what is displayed on the application during the encounter. The ability to block screen captures can provide compliance with some aspects of HIPAA. The application can block or prevent the operating software of the mobile device from screen captures while the application is running.

The application and UI can be compatible to display, record, and transmit images in different file formats, such as DICOM, jpeg, and other image storage formats.

The application and backend can allow for an administrator, such as a hospital administrator to manage the access to the application. For example, there can be a relatively high turnover or churn in emergency room groups. The administrator can manage the access list to update the list of physicians in the emergency room to coincide with the current roster of emergency room physicians.

The application and UI can include the ability to verify and authenticate an adapter or lens that is used with the mobile device. The application can verify the adapter, such as verifying the adapter hardware. The application can then contact a remote computer network to verify if the use of the adapter hardware requires a license and whether the user is authorized to use the adapter hardware.

The application and UI can include the ability to input a touch ID. The touch ID can include a finger print or thumb print for verification of the patient or user (e.g. physician collecting the patient data point). A finger print scanner on the mobile device can be used to input the finger print. For example, a patient finger scan can be used to sign a consent form within the application. This can eliminate a signature on hard copies of the paperwork and speed up and streamline the overall examination process. The application and UI can also provide for the ability to the patient or physician to input an electronic signature, similar to Docusign. The physician can review the examination results from the encounter and provide an e-signature to sign for the results. The ability for a signature to be provided electronically can speed up the examination process and also satisfy requirements for reimbursements.

The application and UI can provide the physician with a list of doctors that are currently on call.

The application and UI can provide a reminder to the physician to close out the encounter so that data recorded during the encounter can be uploaded to EHR.

The application can automatically record messages associated with the referral communications between the referring physician and the ophthalmologist reviewing the patient data points and providing an assessment of the patient. Under current conventional electronic health record practice these communications are not added to the EHR unless the physician separately types this information into the system, which is time consuming and laborious. The application can automatically include this information in the EHR, which can increase the accuracy and improve the information in the EHR.

The application and backend can record when and who views images of patient data. The recording and tracking of who and when images are viewed complies with portions of HIPAA.

The application and UI can allow the physician the ability to calculate the tear breakup time of the patient.

The application and UI can provide a button to automatically send a fax to a colleague, referring physician, or other healthcare person. Fax machines rely on an older technology and are time consuming to use to transmit information. The ability to send a fax directly through the application can save physician time.

The application and UI can provide a summary of referral information to the user (physician obtaining the patient data point or the ophthalmologist providing the assessment). The referral information can include a referral score card that shows the referral sources (OD, PCP, cornea specialist, etc.) along with the frequency of different disease states either referred or assessed.

The application and UI can provide an ambient light indication to the user of the mobile device. For example, an ambient light sensor on the mobile device can measure the ambient light and the application can receive that data and provide an indication to the user as to the level of ambient light. If the ambient light is bright, such as on a sunny day outside, then the application can provide notice to the user that the ambient light may be too bright.

The application, UI, and back end can be used to keep various databases separate and manage user access. For example, a hospital can have multiple locations with many different physicians. The access can be limited for physicians based on their location so they can't access records for patients at other locations. The application is generally designed for physician and healthcare provider use with the patient not having direct access to timeline and medical records in the application. The different referral lists can be managed to send to a subset of doctors for each physician collecting patient data points. The physician can have multiple different lists with ophthalmologists. The referral list is not shown to the ophthalmologists on the referral list. It can be important to avoid doctors that receive patient referral requests to see the contact information or other doctors that receive referrals. The application can also require an authentication module to control access to patient data and to determine whether the user is authorized to provide a certain action (collect patient data point, provide assessment, etc.).

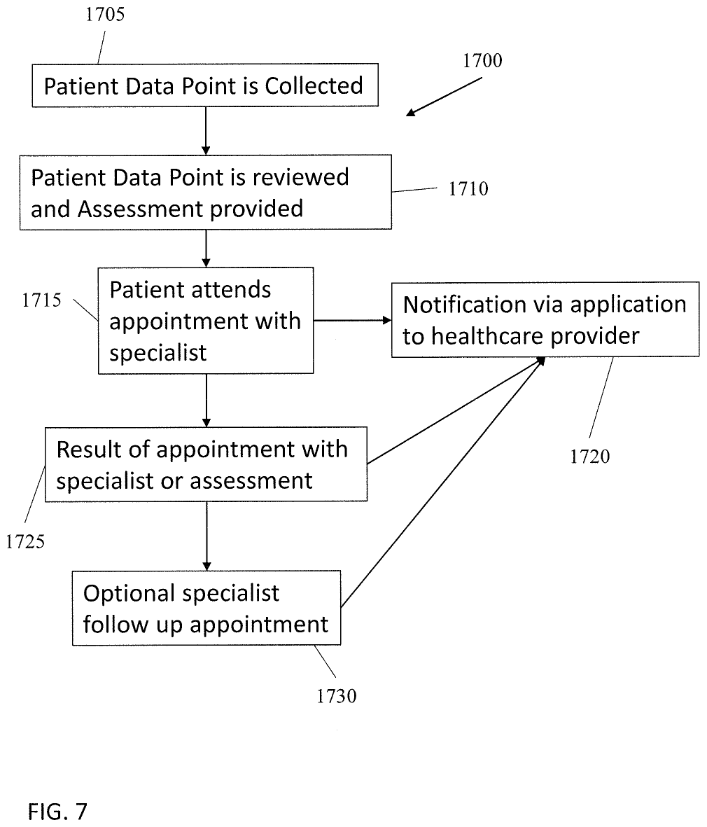

The application can be used to provide notifications to the users of the application and track patient progress. The notifications can be useful for the healthcare provider that sees the patient and collects the patient data point. For example, the patient can get the initial treatment at a hospital that uses a first standard computer system for handling medical records. The assessment can suggest the need for the patient to get treatment by a specialist or ophthalmologist that is outside of the hospital, such as a private practice that uses a second standard computer system for handling medical records. The first standard computer system and second standard computer system may not communicate directly so an employee would have to follow up to see if and when the patient visited the specialist. Under current electronic medical systems and practices there is no automatic way to keep track of all of the referrals and the results. The application and methods described herein can keep track of the patient events like seeing the specialist/ophthalmologist, the result/assessment of the appointment, the need for follow up, and scheduling/results of any follow up appointments. The application can send notifications of the occurrence of any of these events to the referring physician. The update can be used for the physician to satisfy additional reimbursement conditions such as providing medical services and quality care metrics. FIG. 7 illustrates an exemplary embodiment 1700 of the notification and tracking process that can be provided by the application and backend. The patient data point is collected 1705 and the patient data point is reviewed with an assessment provided by the ophthalmologist 1710. The patient can then attend an appointment with a specialist 1715. A notification can be sent to the healthcare provider/physician that collects the patient data point after the patient attends the appointment 1720. The result of the appointment with specialist and/or an assessment 1725 can also trigger a notification via the application to the healthcare provider. Optional specialist follow up appointments 1730 can also trigger a notification via the application to the healthcare provider.

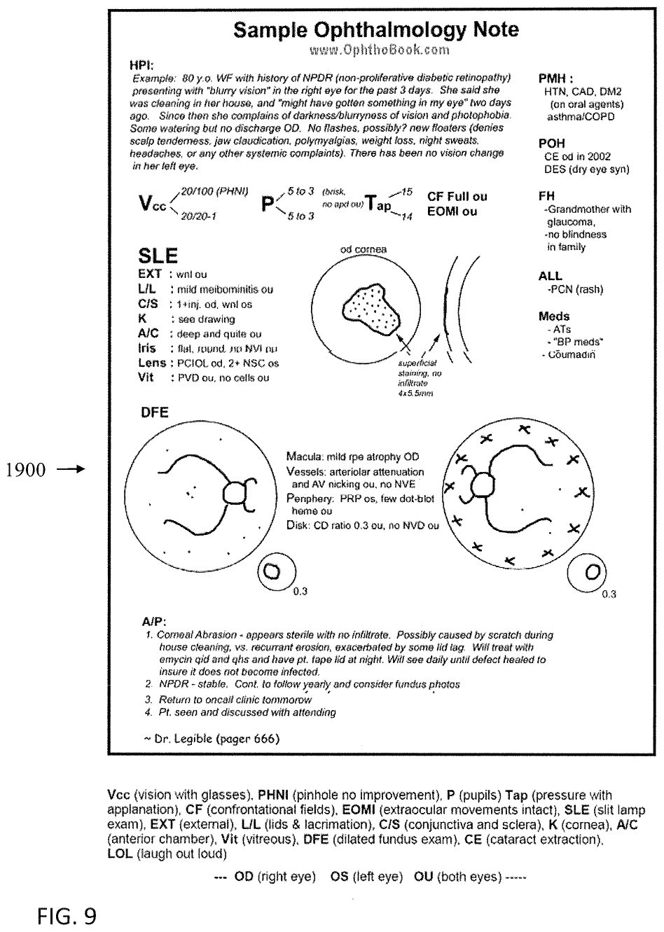

The application and back end can also be used to automatically generate various reports. In one example a note is automatically generated based on the collection of the patient data point done by the initial physician. The note goes to the ophthalmologist reviewing the note and patient data point to provide the assessment. An example of information that can be included in the note includes: Name, patient ID, history, eye pressure, photos, assessment of the description of what is bothering the patient, and any relevant supplemental information. Medical history/medications can also be included in the note. The report can include images of the patient anatomy, when appropriate. In some cases a graphical history of the patient's past examination results, when appropriate. After the ophthalmologist provides the comments/assessment a second report can be automatically prepared that includes the assessment. In some cases the note that is generated is a SOAP note (Subjective, Objective, Assessment and Plan). An example of a sample ophthalmology assessment/SOAP/note 1900 that can be generated based on the information in the assessment is shown in FIG. 9. The sample ophthalmology assessment/note 1900 can include any of the information shown in FIG. 9. In some embodiments the sample ophthalmology assessment/note 1900 includes a family history, a patient symptom, a patient medication, an image of the retina, an image of an anterior segment of the eye, a visual acuity of the patient, an intraocular pressure of the patient, an afferent defect of the patient, a corneal abrasion of the patient, other eye examination data associated with the patient, and one or more comments from the ophthalmologist. The application can apply natural language processing (NLP) to evaluate the notes added by the healthcare provider. The NPL analysis can be used to associate or tag the healthcare providers description of the patient with specific diagnoses or conditions.

The patient timeline can also be used to automatically generate reports. Data within the timeline can be tagged with relevant markers automatically or by the physician with tags to organize subjective and objective items.

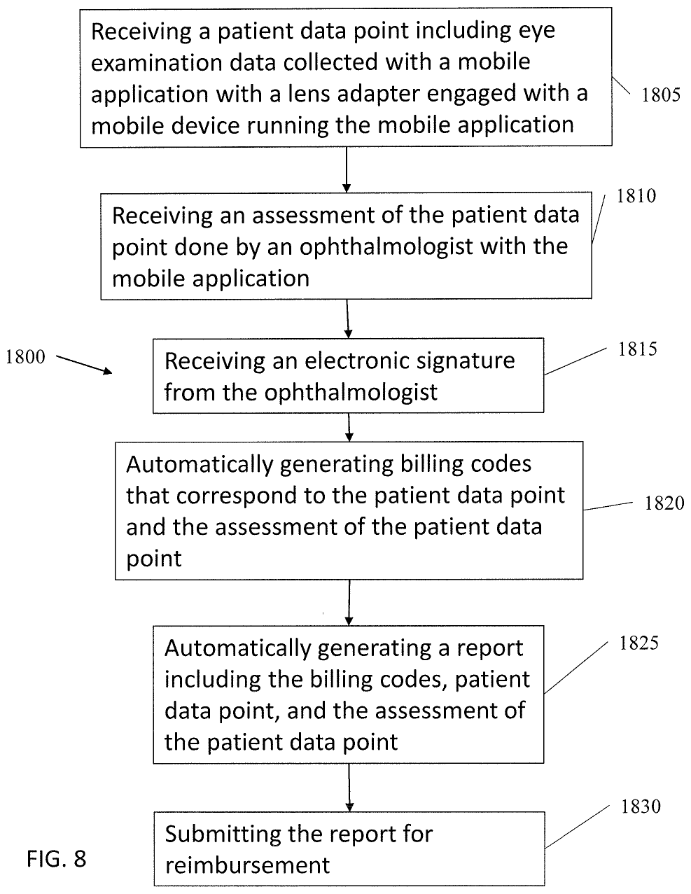

The application and back end can also prepare a report for reimbursement and billing purposes. The automatic preparation of the reimbursement report can save a lot of physician time and administrative time for the healthcare provider. FIG. 8 illustrates a method 1800 for generating a report in accordance with some embodiments. The patient data point is created. The methods can include receiving a patient data point including eye examination data collected with a mobile application with a lens adapter engaged with a mobile device running the mobile application 1805. The ophthalmologist reviews the patient data point and provides an assessment. The methods can include receiving an assessment of the patient data point done by an ophthalmologist with the mobile application 1810. The ophthalmologist can electronically sign the assessment. The methods can include receiving an electronic signature from the ophthalmologist 1815. The methods can include automatically generating billing codes that correspond to the patient data point and the assessment of the patient data point 1820. The assessment can be reviewed such that billing codes, like ICD or CPT codes, are automatically selected based on the patient data point and assessment. Examples of non-limiting relevant CPT codes for new patients include 92002 for ophthalmological services (medical examination and evaluation with initiation of diagnostic and treatment program; intermediate, new patient) and 92004 for ophthalmological services (medical examination and evaluation with initiation of diagnostic and treatment program; comprehensive, new patient, one or more visits). Examples of non-limiting relevant CPT codes for established patients include: 92012 for ophthalmological services (medical examination and evaluation, with initiation or continuation of diagnostic and treatment program; intermediate, established patient) and 92014 for ophthalmological services (medical examination and evaluation, with initiation or continuation of diagnostic and treatment program; comprehensive, established patient, one or more visits). The methods can include automatically generating a report including the billing codes, patient data point, and the assessment of the patient data point 1825. The report can include the patient history, general medical observation, external examination, gross visual fields, basic sensorimotor evaluation, ophthalmoscopic examination, and other results of the assessment and patient data point collection. The automatically generated report can be designed to satisfy reimbursement requirements. The report can then be submitted for reimbursement to the insurance provider. The methods can also include submitting the report for reimbursement 1830. In some embodiments the patient data point is collected by a non-ophthalmologist. The patient examination data can include one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, and other eye examination data associated with the patient. The assessment of the patient data point done by the ophthalmologist can includes one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, other eye examination data associated with the patient, and one or more comments from the ophthalmologist. The image of the portion of the eye of the patient can include an image of a retina of the patient obtained with an indirect lens engaged with the lens adapter. The image of the retina can be obtained using any of the methods described herein. The image of the portion of the eye of the patient can include an image of an anterior segment of the eye of the patient obtained with a macro lens of the lens adapter. The image of the anterior segment can be obtained using any of the methods described herein.

The application, UI, and/or mobile device can provide instructions and/or in application tutorials to the physician to use the mobile device to acquire the patient data point for any of the tests described herein. Some of the tests for acquiring the patient data points described herein are typically done by an ophthalmologist and may require some level of skill to quickly and efficiently obtain a useful result. When the physician collecting the patient data point is not an ophthalmologist and has less experience performing the test it can be helpful to provide instructions to properly perform the test. In the example of obtaining a retinal image the mobile device can provide instructions to the user to dilate the pupil of the test subject and instructions for engaging an adapter with an ophthalmoscope lens with the mobile device. The instructions can include how to line up an optical axis of the camera on the mobile device with an optical axis of the ophthalmoscope lens on the adapter. Instructions can be provided to adjust a telescoping feature of the adapter to improve the focus of the ophthalmoscope lens. Instructions can also be provided to the user about how to position and line up the axis and lens to obtain a useful image of the retina. The application can also utilize any of the imaging techniques described herein. For example, the auto capture feature can be used to analyze images recorded in the video feed of the ophthalmoscope lens followed by automatically recording an image of the retina that satisfies a predetermined quality criteria. The physician using the device would not need to hit the camera button but simply follow the instructions from the application to successfully position the device until a suitable image is captured. The instructions can be any combination of visual and auditory instructions. Visual instructions, such as arrows or a positioning guide like lines to line up can be displayed on the display of the mobile device. Auditory instructions can be provided via a speaker of the mobile device. Combinations of auditory instructions and visual instructions can also be provided.

The application can perform a number of different image taking features that can improve the efficiency and quality of the collection and entry of a patient data point. The application can automatically invert the image when the retina is in view of the camera on the mobile device. For the retinal imaging/posterior imaging of the eye the lens that is typically used to obtain this image presents an inverted image. The application can automatically invert the image that is presented on the display of the smartphone during the examination. For example, if the lens moves to the left the image on the display moves to the left.

The application can analyze a video stream (or burst of images) of the eye of the patient and automatically select an image based on a predefined characteristics that are desirable for the image being captured of the eye. This feature can be referred to as auto capture. Auto capture can be particularly useful for acquisition of images during examinations where it may difficult for the physician to have a free hand or digit to press a button on the mobile device to cause the camera to take a picture. For example, when trying to take an image of a retina of the patient the physician may use one hand to secure the lens adjacent to the eye of the patient and another hand to hold the mobile imaging device. It can be difficult to grip the mobile device and then manipulate a free digit to successfully hit a button on the mobile device to take a picture. There can also be a lag between the physician's recognition of a good image of the patient's eye and successfully hitting the button to take the picture.

The application can record a video stream during the examination process and collection of the patient data point. The video stream can be recorded for a short time period around 60 seconds or less, around 30 seconds or less, around 20 seconds or less, around 15 seconds or less, around 10 seconds or less, around 5 seconds or less. The physician can review the video stream to pick the desired image from the video stream to send to the backend. In some cases the video stream can be used to account for a delay in the physician pushing the photo button after seeing the desired image of the eye. The video stream can be on a short loop recording and storing the previous 5-10 seconds of images in a buffer with an image can be presented that is recorded a short time period prior to the camera photo button being pushed. The delay can correspond to the typical time that it takes for a physician to hit the camera button after deciding to take an image. For example, the delay can be less than about 0.5 seconds, 0.25 seconds, or between about 0.10 and 0.5 seconds.

The application and UI can provide image capture and processing features. For example, the application can analyze multiple images and stich the images together to provide an image of the anatomy of interest with a panorama or montage feature. In some cases the stitched image can be combined to improve the resolution of the processed image versus each individual image taken by the camera of the mobile device. The application can combine multiple images to improve the overall image resolution using various digital image processing techniques, including: filtering, edge detection, skeletonization, thresholding, etc. A combined image of the eye can be prepared from the plurality of images through applying the digital image processing techniques to the plurality of images. In some cases the application can select a best focused image from a plurality of images acquired by the camera of the mobile device either in a burst mode or video mode.

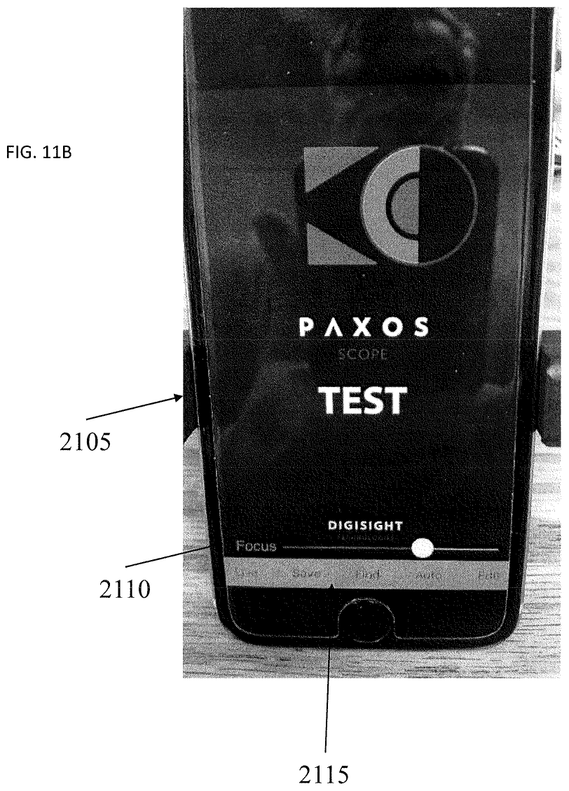

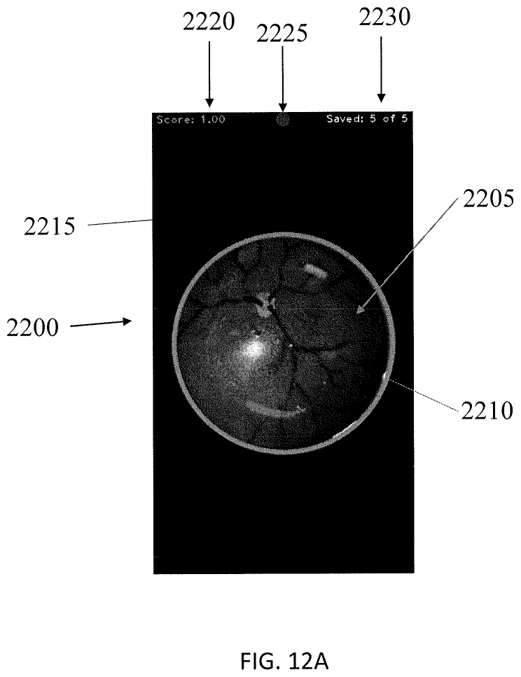

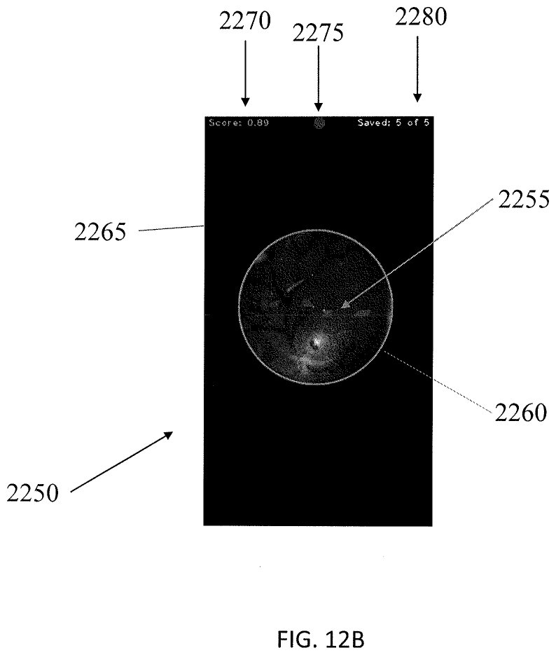

The application can mask portions of the images of the fundus. The portions of the image can be automatically masked to remove non-relevant portions of the images, such as images of surrounding anatomy or surrounding items. In some cases the masking feature can be applied to a unique portion of the anatomy that could be used to identify a patient. In other cases portions of the image could be masked to reduce the file size of the image. Examples of masked images where images outside of the contour of the lens or the image of the retina are masked are shown in FIGS. 12A-12B.

The application can modify the autofocus of a live feed of images to improve the quality of the image. The autofocus can be adjusted based on the type of lens being used with the camera of the mobile imaging device. The focus of the device can be automatically adjusted to be the best focus based on the type of lenses being used, which can be automatically detected by the mobile imaging device, and the image being acquired of the patient anatomy.

The exposure, focus, and zoom can also be set such that if the image detects the presence of an image of the retina then the exposure, focus, and zoom can automatically be adjusted to zoom in on the retina to a predetermined level and to improve the exposure and focus on the retina to further improve the image of the retina. In some cases the exposure, focus, and zoom optimization can be activated by voice control or by tapping on a portion of the screen.

The application can analyze the patient data point or image of the patient anatomy to automatically identify anatomical features of the patient anatomy. For example, the images can be analyzed using machine learning, artificial intelligence, neural networks, and the like to identify desired anatomical features of interest or indications of diseased tissue. In one example the patient data point can be analyzed shortly after acquisition to provide an indication to the physician collecting the patient data point (and subsequent ophthalmologist reviewing the patient data point to provide an assessment) whether the image is fine or whether the patient data point may include an image of a disease or possible health problem. The application could display a green light or thumbs up if the machine analysis of the image indicates that the image may not contain evidence of an eye or health problem. The application could display a red light or thumbs down if the machine analysis of the image indicates that the image may contain evidence of an eye or health problem.

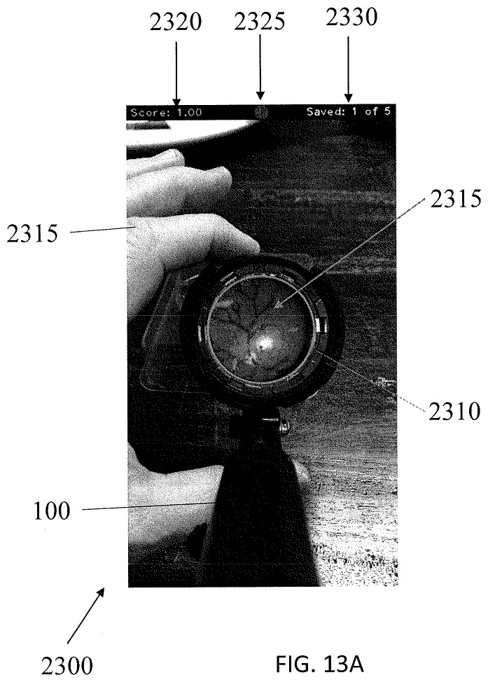

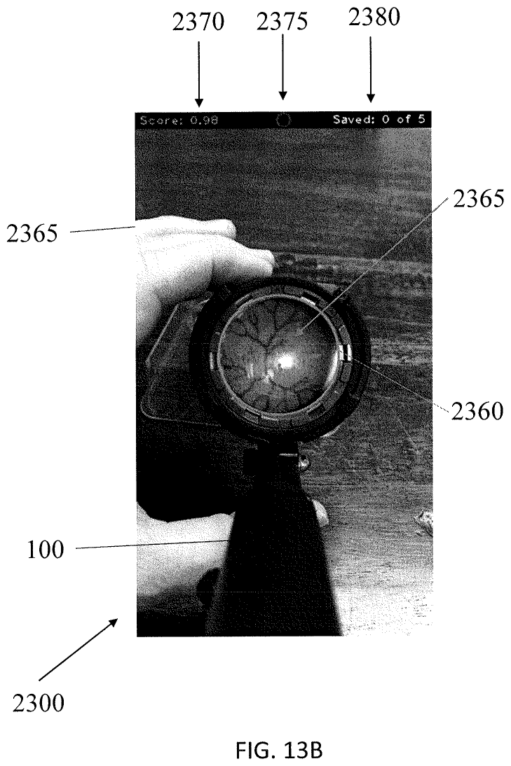

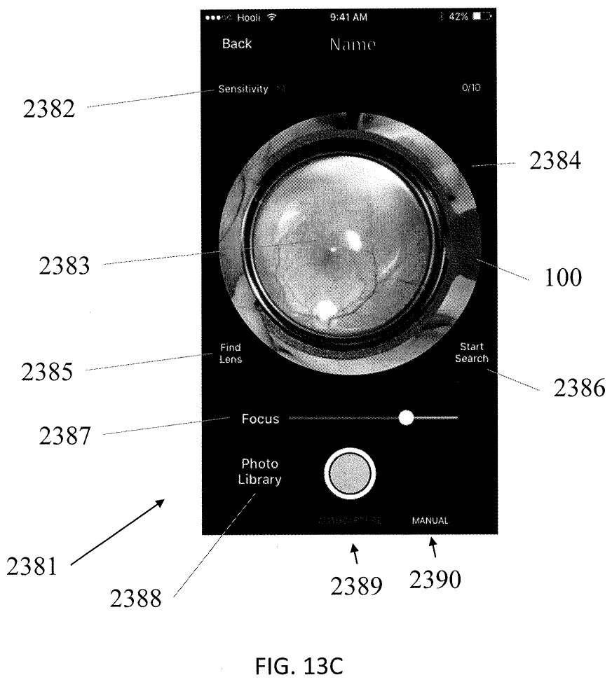

The application can analyze the image to determine whether the image is complete and of a sufficient quality for further analysis. The analysis of the quality of the image of the retina can be a quantitative score. The quantitative quality score can correspond to a determination by a software algorithm that can utilize computer vision or other image analysis to determine the quality of the image of the retina. The algorithm can factor in the image of the retina and compare it to an image of an ideal retina. The algorithm can also analyze the image for the presence or absence of glare, poor light, overexposure, blur, poor focus, and other image related artefacts and include that data in the quality score. A higher quality score indicates a higher quality image. The quality score defined by the application varies from 0 to 1, with 1.00 being the highest score. FIGS. 12A, 12B, 13A, and 13B illustrate images of retinas along with the respective quality scores. In some embodiments the indication of the quality of the image can be provided by a quantitative score shown on the UI. In some cases the quality of the image can be shown by providing an indication to the user such as by changing a color of a portion or area on the UI. For example, a color could change on a portion of the UI. In one example an outline of a contour of the indirect lens can be shown on the UI with the color of the outline indicating whether the quality score is above a predetermined threshold value.

The application can automatically modify the image and/or properties of the camera (light, shutter speed, exposure, etc.) when acquiring the image to filter, remove, or minimize glare.

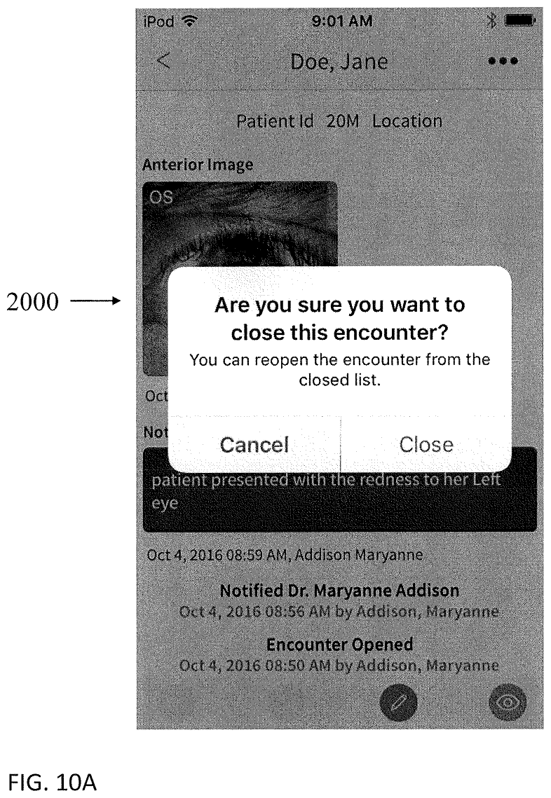

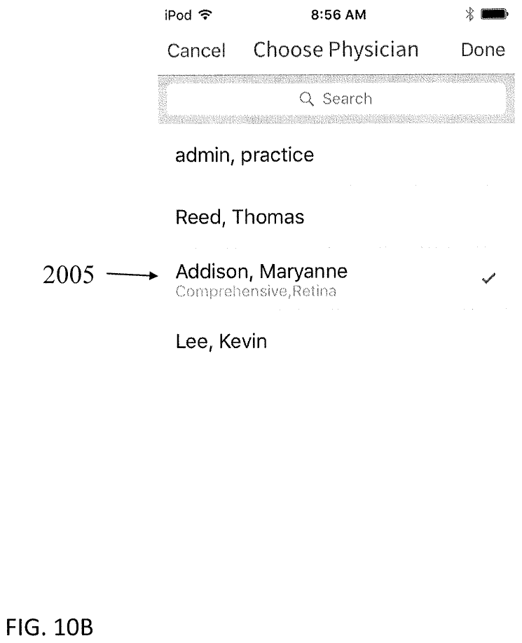

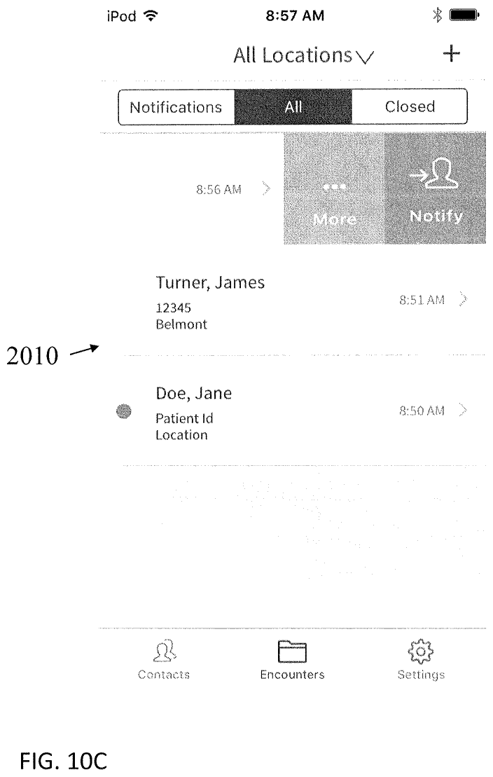

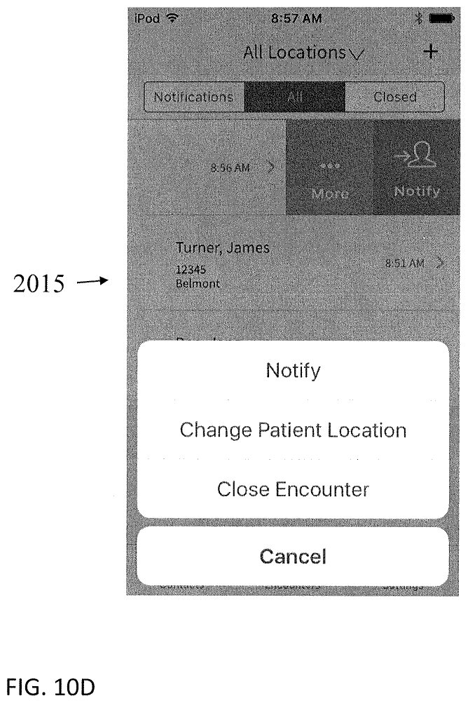

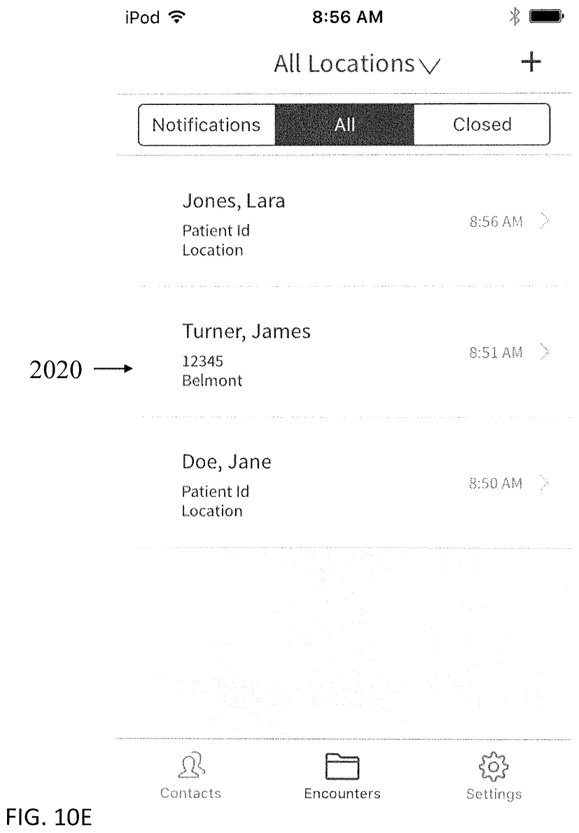

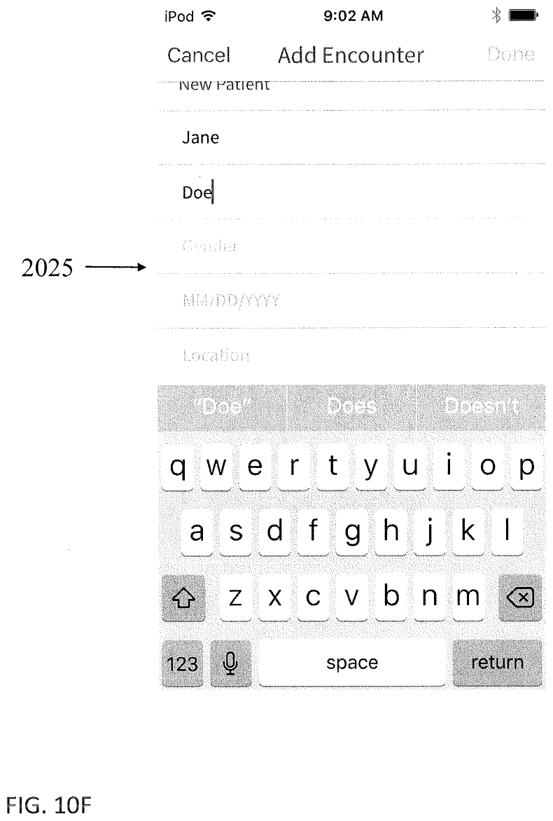

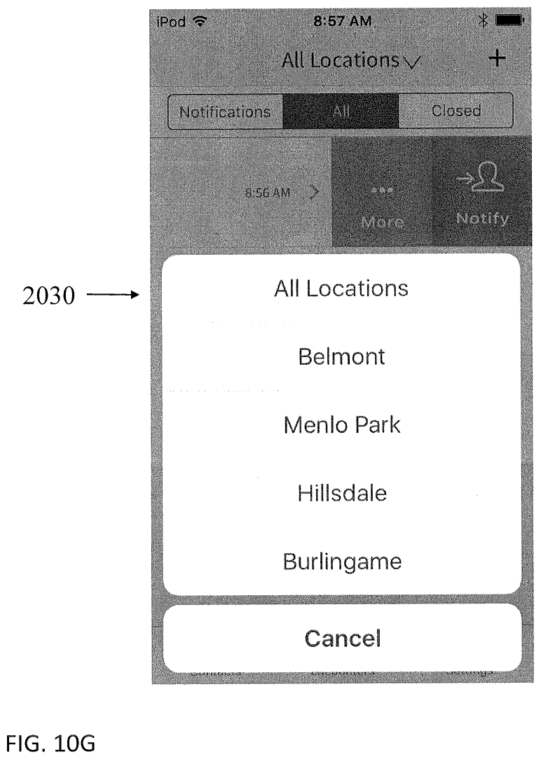

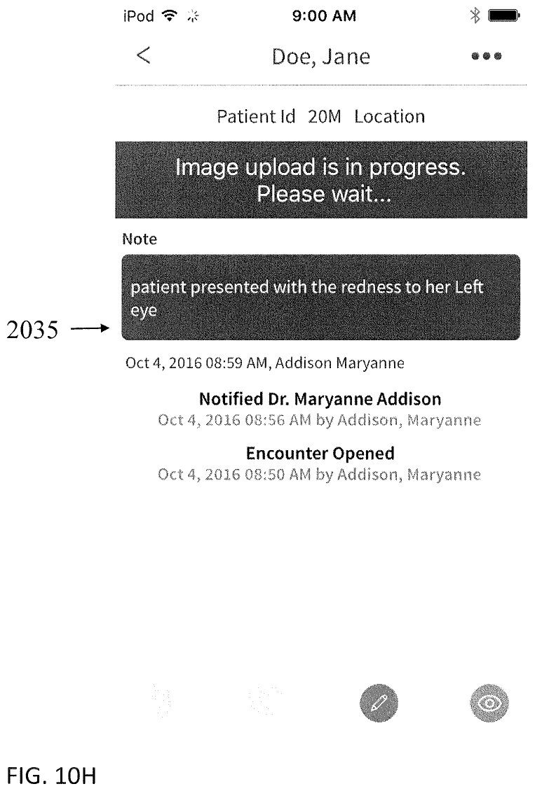

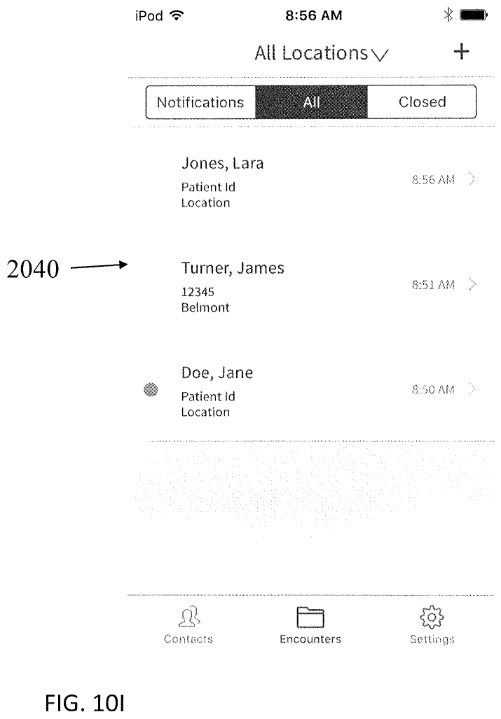

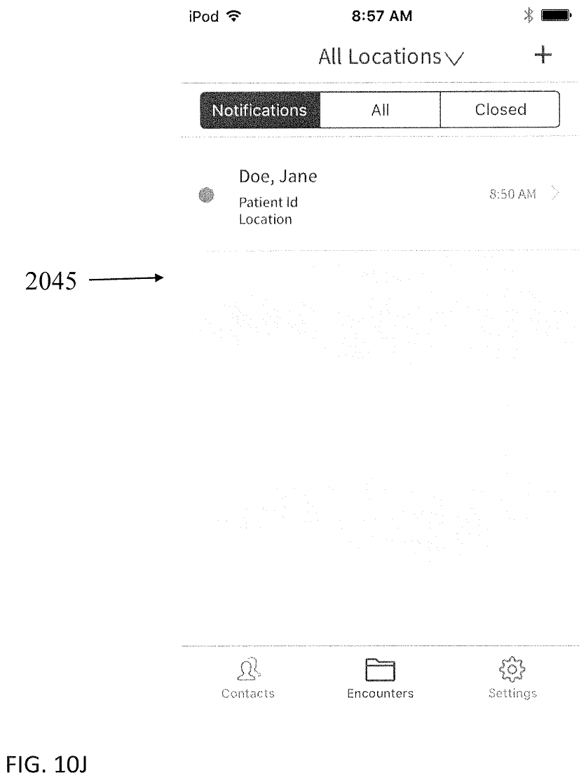

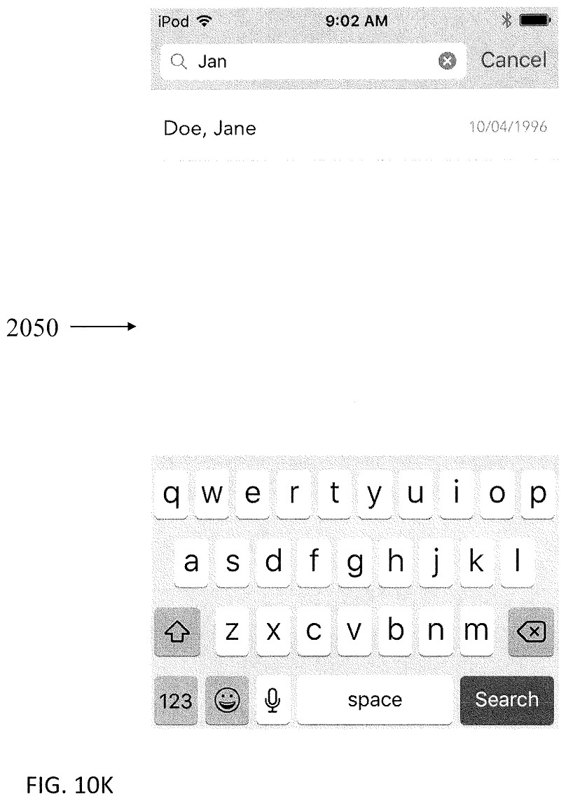

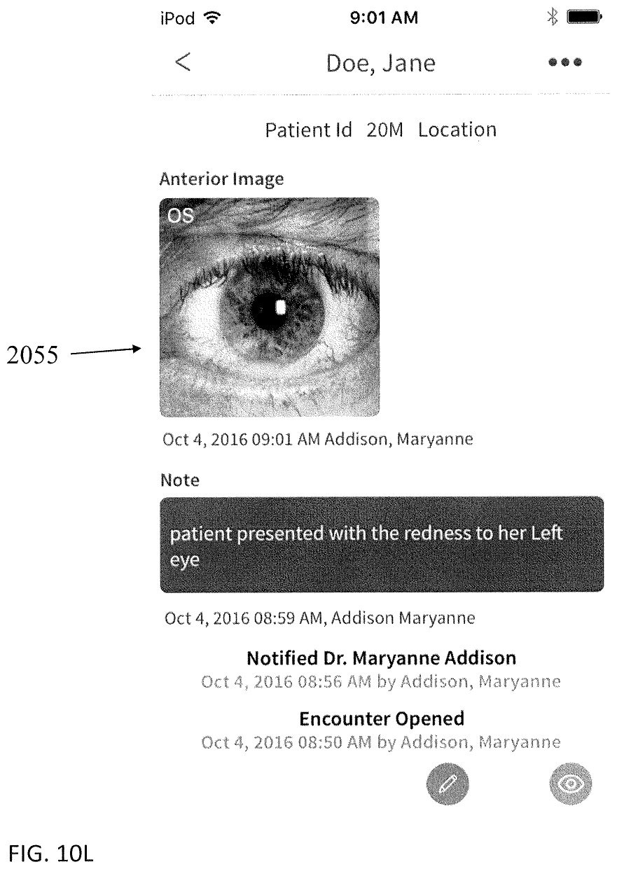

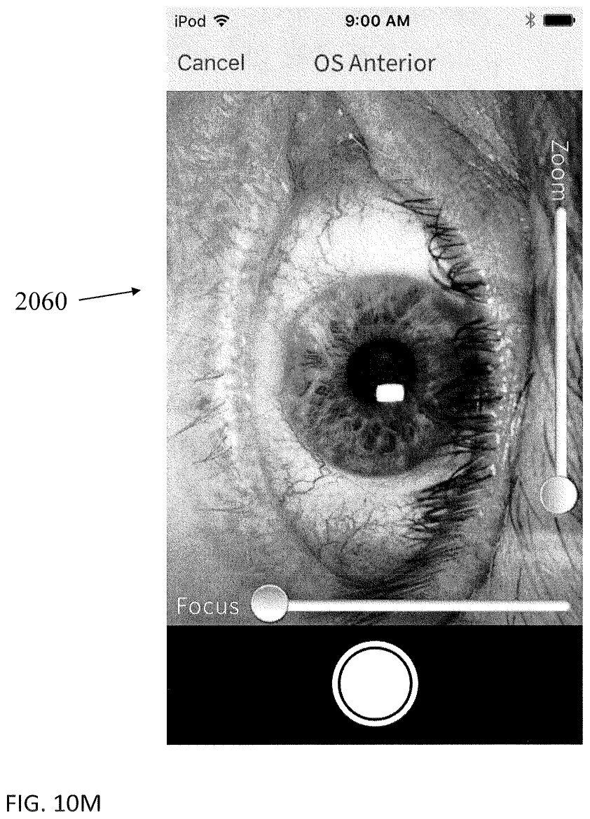

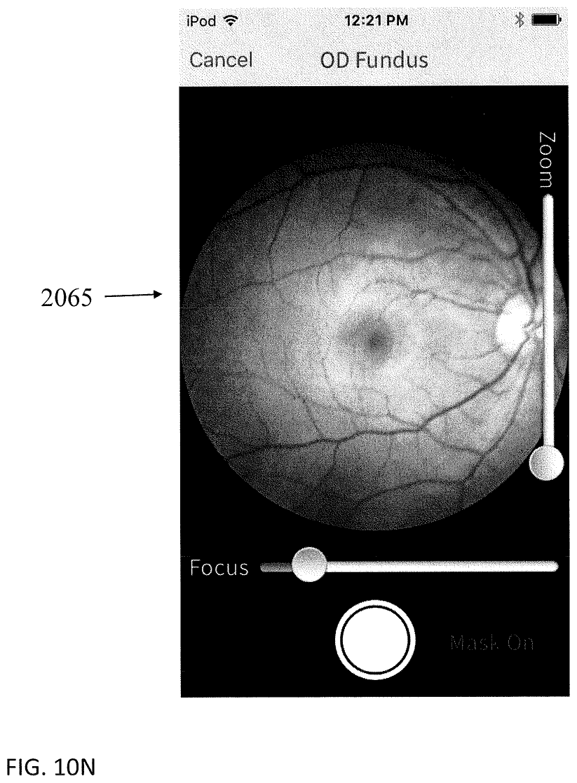

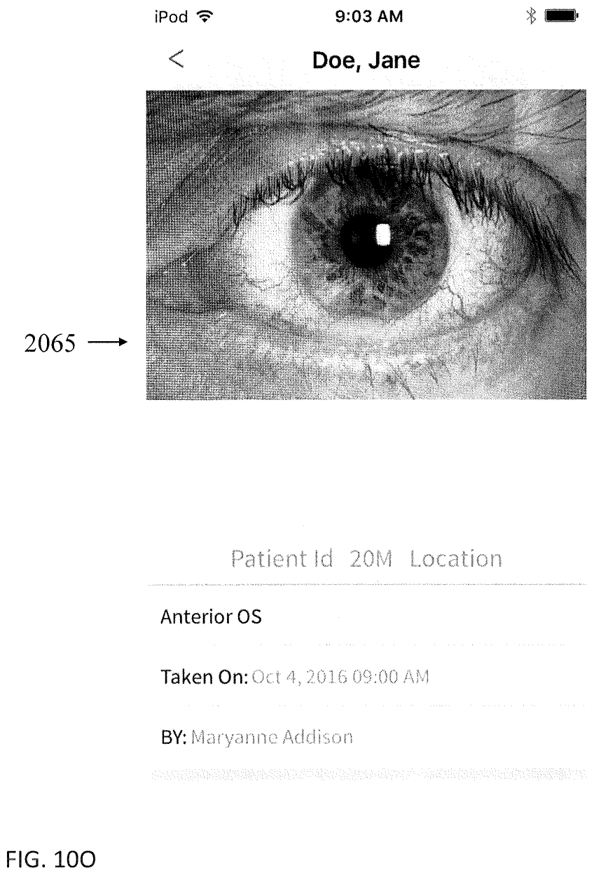

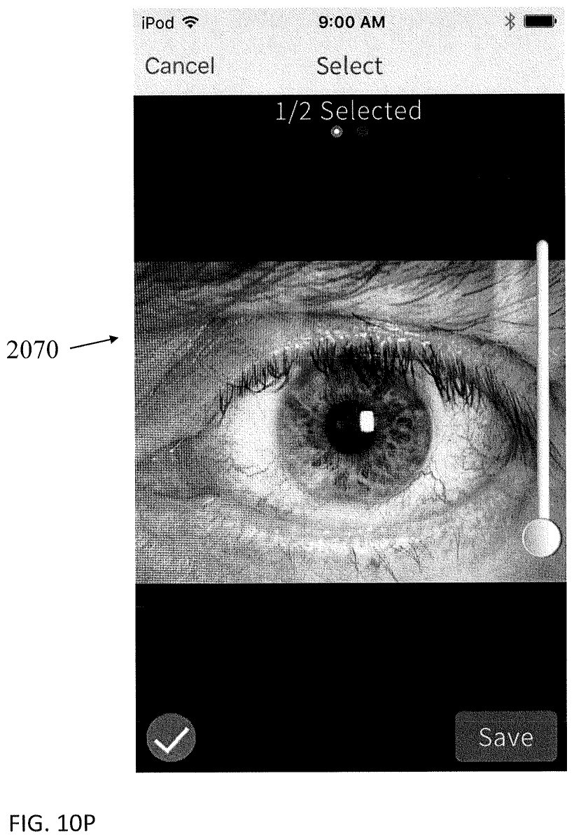

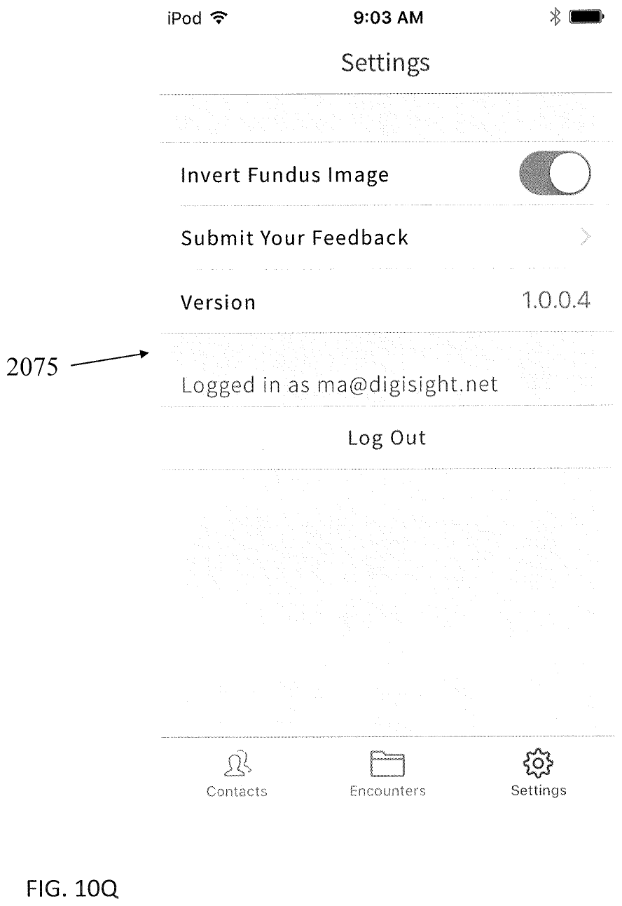

FIG. 10A illustrates an example of a screen shot 2000 of an application UI showing a prompt for closing an encounter after collecting an anterior image of the eye and providing notes on the patient to the application during the eye examination and patient data point collection.

FIG. 10B illustrates an example of a screen shot 2005 of an application UI showing a contact list for a user of the application. The user can select a specific physician or group of physicians to send a message, referral request, or other communication.

FIG. 10E illustrates an example of a screen shot 2020 of an application UI showing a listing of encounters. FIG. 10C illustrates an example of a screen shot 2010 of an application UI showing a tab listing encounters with options. At the top of the UI shows that the encounters are listed for all locations and also includes tabs to navigate between notifications, all encounters, and closed encounters. Sliding left on an encounter reveals several options include a “Notify” feature and “more” feature with which to use to manipulate or process the encounter.

FIG. 10D illustrates an example a screen shot 2015 of an application UI showing encounter processing options that can pop up on the screen of the mobile device, including notify, change patient location, and close encounter.

FIG. 10F illustrates an example of a screen shot 2025 of an application UI showing a prompt that can be used to add a patient and/or an encounter. The UI shows patient information in FIG. 10F.

FIG. 10G illustrates an example of a screen shot 2030 of an application UI showing a prompt listing various location tags that can be used to label the patient location by the healthcare provider.

FIG. 10H illustrates an example of a screen shot 2035 of an application UI showing notes that have been added by the physician (non-ophthalmologist) during an encounter with the patient that includes taking an image of the patient's eye.

FIG. 10I illustrates an example of a screen shot 2040 of an application UI showing an encounter list with a notification dot next to the “Doe, Jane” encounter. The notification provides an updated message, image, or other relevant information has been added to the encounter.

FIG. 10J illustrates an example of a screen shot 2045 of an application UI showing an encounter list with a notification dot next to the “Doe, Jane” encounter through the “Notification” tab of the encounter listing. The notification provides an updated message, image, or other relevant information has been added to the encounter.

FIG. 10K illustrates an example of a screen shot 2050 of an application UI showing a search box being used to search for a patient within the application along with a preliminary search result showing a record for “Doe, Jane.”

FIG. 10L illustrates an example of a screen shot 2055 of an application UI showing an example of the patient timeline including an image of the anterior segment of the patient's eye and notes inputted by the non-ophthalmologist examining the patient. The UI interface includes buttons at the bottom for adding a note to the timeline or image to the timeline.

FIG. 10M illustrates an example of a screen shot 2060 of an application UI showing an image acquisition module for the anterior segment of the patient's eye. The UI shows the real time image of the anterior portion of the patient's eye along with a photo button, focus slide adjuster, and zoom slide adjuster that can be used to improve the acquisition of a high quality image of the anterior portion of the eye.

FIG. 10N illustrates an example of a screen shot 2065 of an application UI showing an image acquisition module for a posterior segment of the patient's eye. The UI shows the real time image of the posterior portion of the patient's eye along with a photo button, focus slide adjuster, and zoom slide adjuster that can be used to improve the acquisition of a high quality image of the posterior portion of the eye. The UI also indicates that a mask feature is on to block out extraneous anatomy and images such that the posterior image of the eye is all that is shown on the UI.

FIG. 10O illustrates an example of a screen shot 2070 of an application UI showing an image of the anterior segment of the patient's eye along with identifying information for when and who took the photo.

FIG. 10P illustrates an example of a screen shot 2075 of an application UI showing a part of the photo selection process that can be used to pick the best image of the anterior segment of the patient's eye. The user can toggle or slide between multiple images taken in a camera burst mode or video mode to select the highest quality or best image of the anterior portion of the eye. After the user selects the desired image the image can be saved and added to the patient encounter.

FIG. 10Q illustrates an example of a screen shot 2080 of an application UI showing a settings page for the application. The UI indicates who is signed in to the application, the version of the application, along with an invert fundus option. The switch can be toggled between an invert fundus mode and a regular fundus mode.

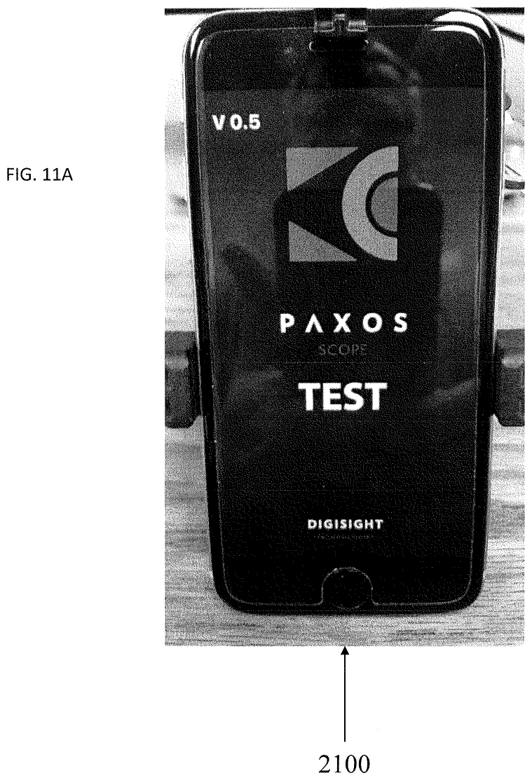

FIGS. 11A-11B illustrate examples of screen shots 2100, 2105 of an application with a user interface (UI) on a mobile device in accordance with some embodiments. FIGS. 11A-11B illustrate examples of a UI 2100, 2105 for a splash screen of the application on the mobile device. FIG. 11B shows a focus slider bar 2110 at the bottom of the UI. The focus slider bar 2110 can be used to focus the camera of the mobile device on a portion of the live image displayed on the UI/display of the UI. The focus slider 2110 can be used to manually set the focus of the camera. Focus can also be automatically done by tapping on a portion of the live image for the camera to automatically focus on that spot. FIG. 11B also shows a menu 2115 with buttons on the bottom of the UI, including: live, save, find, auto, and edit buttons. The user of the mobile device can select between the different modes using the buttons at the bottom of the UI as shown in FIG. 11B.

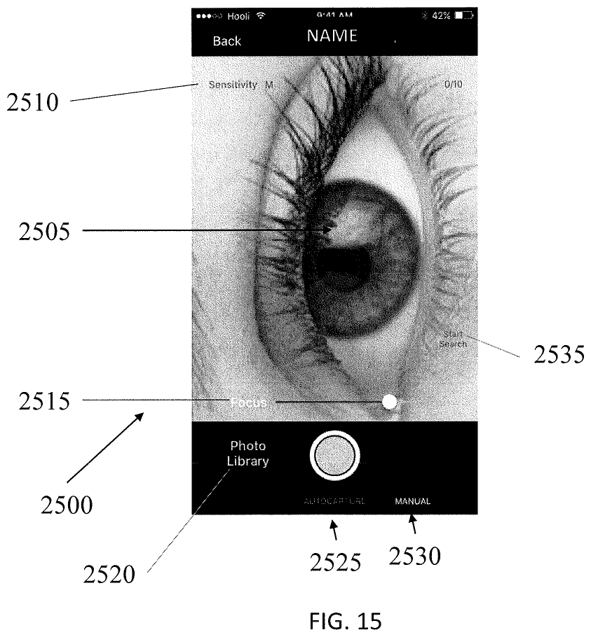

FIGS. 12A-12B illustrate examples of screen shots 2200, 2250 of an application showing an image of a portion of a retina 2205, 2255 on a mobile device in accordance with some embodiments. The images of the retina displayed in FIGS. 12A-12B show an image of the retina 2205, 2255 taken through the external lens adapter inside of a circular lens contour 2210, 2260 illustrated as a circle on the display. FIGS. 13A-13B illustrate images obtained from the camera of the mobile device of a model of a retina 2315, 2365. In FIGS. 12A-12B a mask 2215, 2265 is applied to remove a portion of the image from the camera of the mobile device that is outside of the contour or circle 2210, 2260 defined by the external lens. The illustrated mask 2215, 2265 is a black or darkly colored mask. FIGS. 13A-13B illustrate images from the camera that are displayed without applying the mask, thereby showing the areas 2315, 2365 outside of the lens contour 2310, 2360. In FIGS. 13A-13B the lens adapter 100 is visible without the mask applied. The images of the UI shown in FIGS. 12A-12B and 13A-13B display additional information relating to the image of the retina, including the quality score 2220, 2270, 2320, 2370 of the image of the retina in the top left corner, an indicator of whether the image was auto captured or manually captured 2225, 2275, 2325, 2375 in the top middle edge of the UI, and the image number and saved status 2230, 2280, 2330, 2380 of the image of the retina in the top right corner of the UI. FIG. 15 illustrates a screen shot 2500 of the UI showing an image of the anterior segment of an eye of a patient.

Auto capture can be used to automatically record images of the eye, including an image of the posterior segment like the retina and images of the anterior segment of the eye. The quality score is a quantitative score that can correspond to the sensitivity of the system to detecting a retina in the image obtained by the camera of the mobile device. The quality score can also be analyzed for an image of the anterior segment of the eye. The quality score can correspond to a determination by a software algorithm that can utilize computer vision or other image analysis to determine the quality of the image of the retina. The algorithm can factor in the image of the retina and compare it to an image of an ideal retina. The algorithm can also analyze the image for the presence or absence of glare, poor light, overexposure, blur, poor focus, and other image related artefacts and include that data in the quality score. A higher quality score indicates a higher quality image. The quality score defined by the application varies from 0 to 1, with 1.00 being the highest score. FIG. 12A displays a quality score 2220 of the image of 1.00. FIG. 12B displays a quality score 2270 of 0.89. FIG. 13A displays a quality score 2320 of 1.00. FIG. 13B displays a quality score 2370 of 0.98.

The images of the retina through the lens of the lens adapter can be automatically captured using the application in an auto capture mode. The images of the anterior segment through a lens of the lens adapter can also be automatically captured using the application in an auto capture mode. The auto capture mode can be turned on by pushing a button on the UI to start the auto capture mode. The auto capture mode can automatically record images of the retina or anterior segment that exceed the predetermined quality threshold. The auto capture mode can be set to capture a predetermined number of images. After the number of predetermined number of images have been taken the images can be automatically saved. In some cases if the predetermined number of images are not obtained then none of the images will be saved. After the full predetermined number of images are captured the user can be prompted to save or clear each of the images in the predetermined number of images. Saving the predetermined number of images can take a couple of seconds or longer depending on the quality and size of the images. In some cases the application and UI can provide a notification that the images were successfully saved. In other cases the application and UI may not provide a notification that the images were successfully saved. After the images have been saved they can be viewed by the user in the photo album. In some cases, when the auto capture mode is activated the UI can deactivate the save button as the images will be automatically captured by the application. As described herein the UI can provide an indicator to the user as to whether the auto capture mode is activated or not. The duration of time between successive images that are captured in the auto capture mode can be set by the user or the application. For example, duration of time between successive images can be selectable from about 1 to about 5 seconds.

The auto capture can also be used in combination with the quality score determination by the application. A pre-determined quality threshold can be set by the user or the application such that the images of the retina or anterior segment are captured by camera of the mobile device once the quality of the image of the retina or anterior segment exceeds the pre-determined quality threshold. For example, if the sensitivity is set to low or a low quality threshold then the system will capture retina or anterior segment images that are not optimal in terms of lighting or even pathology. If the sensitivity is set to high or a high quality threshold then the system will only capture retina or anterior segment images that look like an ideal retina or anterior segment.

The application can display whether the image of the retina or anterior segment was captured manually by the user or using the auto capture functionality. Various symbols can be displayed by the UI to convey information to the user as to whether the image was obtained manually or using auto capture. FIGS. 12A, 12B, and 13A display a red circle 2225, 2275, 2325 that is filled in to indicate that the pictured image was obtained using auto capture. In contrast FIG. 13B illustrates a red circle 2375 that is not filled in to indicate that the image of the retina was obtained manually.

The application and UI can display a circle or ring around the lens to indicate information to the user as to the quality of the image of the retina received by the camera of the mobile device. For example, the circle or ring can appear once the lens of the lens adapter is identified and/or an image of a retina is detected through the lens by the camera of the mobile device. The color or configuration of the lens circle can change to indicate additional information associated with the image obtained by the camera of the mobile device, such as the quality of the image of the retina, zoom/focus, and other details associated with the quality of the image of the retina. In one aspect the color of the lens circle can change to green once the pre-determined quality threshold for the image of the retina has been met. The illustrated lens is a 20 D indirect lens. The algorithm can detect a circle or other shape corresponding to the lens and then apply the corresponding shape, such as the circle, to the image of the lens displayed by the application. Although illustrated as a circle, other shapes can be used to correspond to the lens.

Autofocus or manual focus can be used to obtain the image of the retina or anterior segment. The focus setting can be selected using the menu of the application. In one example, if the lens circle is found in the image of the retina and the zoom is above a threshold, such as greater than about 1, then a single tap on the display will automatically set the focus and the exposure at the center of the lens circle. In another example, the auto focus and auto exposure are set to the portion of the image corresponding to where the display is tapped by the user.

The image can be zoomed in or out. For example, the image at the center of the lens counter can be zoomed in or out by pinching in or out on the display screen. The zoom controls and setting can also be set to achieve a pre-determined zoom scale to achieve a desired image size of the lens and retina. In one example the zoom scale can be automatically set to have the lens circle be about 90% of one of the image dimensions. In some cases the automatic zoom can be triggered upon detection of an image of the lens and/or an image of the retina in the lens. In some cases an optional cross hair display can be selected as well.

After the image is captured, in manual or auto capture mode, it can be saved. The image can be saved as described herein. In one example the image is saved in the local memory. In another example the image can be automatically uploaded to the cloud or other remote network. In some examples the captured images can be first saved locally. Then the user can review the captured images to pick the best image or several best images of the retina. After determining the preferred images the user can select those to be saved and uploaded to the cloud or other remote network.

The UI can display the image number and saved status 2230, 2280, 2330, 2380 of the image of the retina in the top right corner of the UI as shown in FIGS. 12A, 12B, 13A, and 13B. Each of those UI images show the saved status along with the number of images that were saved and the number corresponding to the displayed image. The upper right of the UI shows the image number as 5 images. The number of images that are taken of the retina is selectable through the menu on the edit screen. The user can select the number of images that are to be captured of the retina during the auto capture or manual capture of images of the retina. An example of a menu screen is illustrated in FIG. 11B. The user can review the saved images of the retina to annotate, make notes, or select the best image of the retina for additional analysis or to send to another healthcare provider.

The use of the mask to cover the area outside of the lens can be also be controlled using the UI of the application. For example the menu can be used to turn the mask on and off by the edit screen. In some cases the color and/or pattern of the mask area can be set by the user through the application. The mask functionality can be provided after the contour of the lens is identified in the image obtained by the camera of the mobile device. For example, the mask functionality can be disabled prior to identification of the contour of the lens by the application.

The operation of the camera can also be controlled through the application. For example the menu can be used to toggle between the auto capture mode and manual capture mode. The Auto button can be pressed to toggle Auto capture from ON/OFF. The red circle at the top of the screen is live and immediately updates once the auto button is pressed.

FIG. 13B illustrates the application in manual capture mode (not auto capture mode) as indicated by the hollow circle 2375 at the top middle of the UI. The illustrated UI displays the raw image obtained by the camera of the mobile device. The manual capture mode can be operated by pressing a button or area of the screen to take the image of the retina through the lens of the lens adapter. After capturing the image in the manual capture mode the image of the retina can be saved in the photo album on the mobile device and/or uploaded to the cloud or other remote computer network.

FIG. 13C illustrates another example of a UI 2381 of an application in accordance with some embodiments. The UI shows the sensitivity setting 2382 that corresponds to the quality threshold for the image of the retina. The UI shows with an image of the lens 2383 and lens adapter 100 with a mask 2384 applied to surrounding anatomy. The UI shows a “find lens” button 2385 and a “start search” button 2386. The “find lens” button 2385 can be used to activate the application to search for the contour of the lens. The UI shows a manual focus slider 2387 as well as a “photo library” button 2388 that can be pressed to view captured images. The toggle button between auto capture 2389 and manual capture 2390 is illustrated at the bottom of the UI.

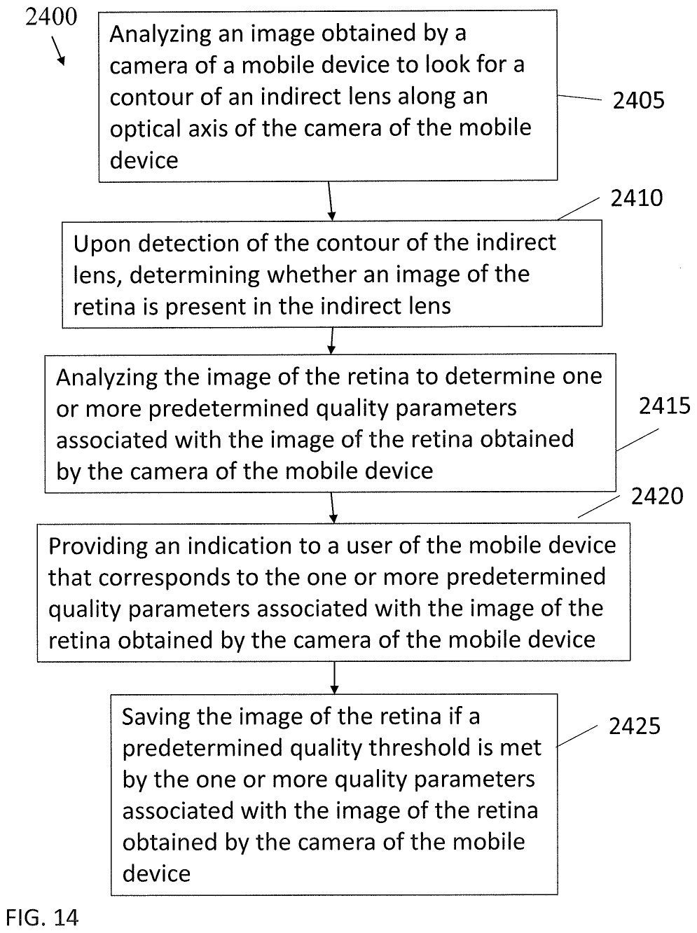

FIG. 14 shows a flow chart of a method 2400 in accordance with some embodiments. The methods can include analyzing an image obtained by a camera of a mobile device to look for a contour of an indirect lens along an optical axis of the camera of the mobile device 2405. Upon detection of the contour of the indirect lens, the method can include determining whether an image of the retina is present in the indirect lens 2410. The methods can include analyzing the image of the retina to determine one or more predetermined quality parameters associated with the image of the retina obtained by the camera of the mobile device 2415. The methods can further include providing an indication to a user of the mobile device that corresponds to the one or more predetermined quality parameters associated with the image of the retina obtained by the camera of the mobile device 2420. The methods can further include saving the image of the retina if a predetermined quality threshold is met by the one or more predetermined quality parameters associated with the image of the retina obtained by the camera of the mobile device 2425. The methods can include engaging the lens adapter with the mobile device. The methods can include dilating the eye of the patient. The methods can include positioning the mobile device and lens adapter such that the lens (e.g. indirect lens) is adjacent to the eye of the patient and an image of the retina is observable through the indirect lens. The methods can include setting a focus setting through the application, such as an automatic focus setting. The methods can include the application applying an outline to the image of the retina or the contour of the lens of the lens adapter. The outline to the image can provide an indication to the user of the mobile device of the quality of the image of the retina. For example the color of the outline of the contour of the lens can be assigned a color that indicates the quality of the image of the retina. The methods can include obscuring or modifying a portion of the image outside of the retina or the contour of the lens to cover, block, or mask the area of the image that is outside of the contour of the lens. The methods can also include capturing a pre-determined number of images of the retina and saving the images after capturing the pre-determined number of images of the retina. The methods can also include providing an indication to the user as to whether the captured images were taken with a manual capture mode or an auto capture mode.

The methods can also include saving the image of the retina if a predetermined quality threshold is met by the one or more quality parameters associated with the image of the retina obtained by the camera of the mobile device. The methods can also include applying a mask to an area of the image outside of the contour of the indirect lens to create a masked image of the retina. The methods can also include displaying the masked image of the retina on a display of the mobile device. In some embodiments the lens contour has a substantially circular shape. In some embodiments the methods further include displaying an inverted image of the retina from the indirect lens on a display of the mobile device.

The methods can also include analyzing a plurality of images of the retina and saving a plurality of images of the retina that meet a predetermined quality threshold. The methods can also include saving the plurality of images of the retina that meet the predetermined quality threshold. In some embodiments the plurality of images of the retina are obtained from a video feed. In some embodiments the plurality of images of the retina are obtained from a multiple pictures taken by the camera of the mobile device. In some embodiments the plurality of images of the retina that meet the predetermined quality threshold includes a predetermined number of images of the retina. In some embodiments the predetermined number of images is 10 or less images of the retina. In some embodiments the predetermined number of images is set by a user of the mobile imaging device. Examples of the one or more predetermined quality parameters associated with the image of the retina include one or more of: glare, exposure, a comparison with an ideal retina image, focus, and lighting.

The methods described herein can include digital image processing. In some embodiments the methods can include analyzing a plurality of images of the retina, applying one or more digital image processing techniques to the plurality of the images of the retina, and forming a combined image of the retina based on the plurality of images of the retina and the applied one or more digital image processing techniques.

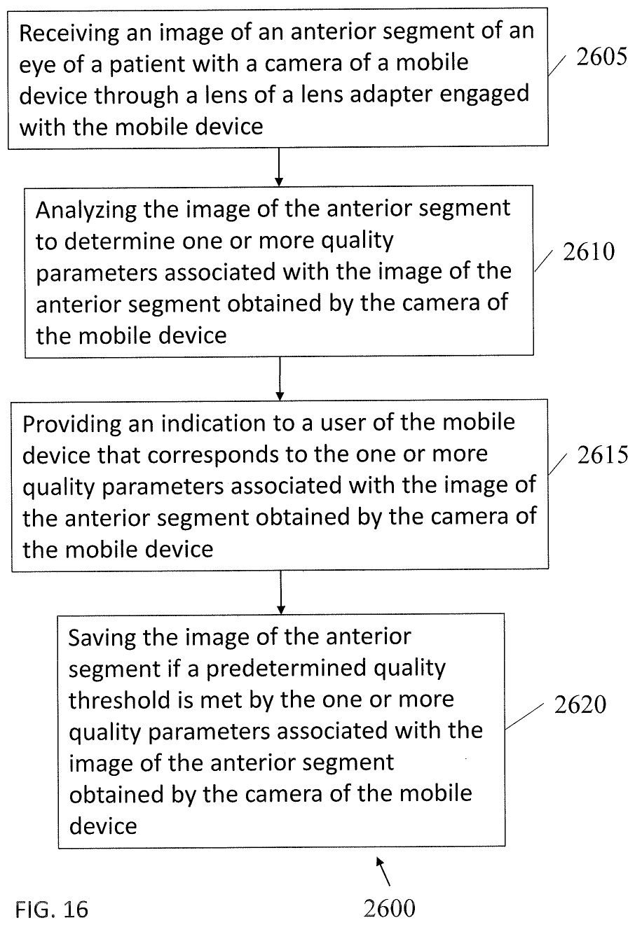

The auto capture processes described herein can also be used to take an image of the anterior segment of the eye of the patient. For example, the lens adapter can include a macro lens that can be positioned adjacent to the camera of the mobile imaging device to obtain an image of the anterior segment of the eye of the patient. A light source of the lens adapter can be used to provide light to the eye to improve the quality of the anterior portion of the eye that is received by the camera of the mobile imaging device. FIG. 15 illustrates examples of screen shots 2500 of an application with a UI on a mobile device in accordance with some embodiments. FIG. 16 shows a flow chart of a method 2600 in accordance with some embodiments.

FIG. 15 shows the UI 2500 displaying an image of the anterior segment 2505 of the patient's eye that can be obtained through a macro lens of the lens adapter. The UI shows the sensitivity setting 2510 that corresponds to the quality threshold for the image of the anterior segment in the top left corner. The UI shows an image the anterior portion of the eye of the patient 2505. Note that a mask is not usually needed as the macro lens and camera of the mobile device are usually positioned relatively close to the eye of the patient such that the image of the anterior segment of the eye takes up a large area of the image received by the camera of the mobile imaging device. The UI shows details about the image number and the total number of images to be recorded by the auto capture mode in the top right of the display. The UI shows a manual focus slider 2515 as well as a “photo library” button 2520 that can be pressed to view captured images. The toggle button between auto capture 2525 and manual capture 2530 is illustrated at the bottom of the UI. A “start search” button 2535 is shown that can be pressed to have the application analyze the image of the anterior segment of the eye to determine the presence of the anterior segment within the image and the quality of the image of the anterior segment.

The auto capture mode for the anterior segment can analyze the quality of the image recorded by the camera of the mobile device. In some embodiments the entire image of the anterior segment can be analyzed to determine the quality score. One aspect of the quality score for the anterior segment is that the algorithm can look for surface eye reflections from a light source of the lens adapter. If surface eye reflections are not detected then the quality score is decreased. If surface eye reflections are detected then the quality score goes up. The decreased quality score from the lack of the eye reflections can remind the user of the application and lens adapter to turn the light source on for the lens adapter. Typically, the quality score will be too low to satisfy the predetermined threshold if the light source is not on and light reflections are not detected. When auto capture is used to obtain the image of the anterior segment of the eye the function is similar to how the posterior images are auto captured. Once the predetermined quality threshold is met then the system can auto capture the images and continue to capture images until the predetermined number of images are obtained.

FIG. 16 shows a flow chart of a method 2600 for obtaining an image of the anterior segment of an eye of a patient in accordance with some embodiments. The method can include receiving an image of an anterior segment of an eye of a patient with a camera of a mobile device through a lens of a lens adapter engaged with the mobile device 2605. The method can include analyzing the image of the anterior segment to determine one or more quality parameters associated with the image of the anterior segment obtained by the camera of the mobile device 2610. The method can optionally include providing an indication to a user of the mobile device that corresponds to the one or more quality parameters associated with the image of the anterior segment obtained by the camera of the mobile device 2615. The method can further include saving the image of the anterior segment if a predetermined quality threshold is met by the one or more quality parameters associated with the image of the anterior segment obtained by the camera of the mobile device 2620. Any of the image processing, saving, annotating, and sending steps described herein can be performed with the images of the anterior segment captured as described herein. In contrast to the auto capture methods used for capturing the posterior segment the anterior segment images do not typically need the use of a mask or the detection of the contour of the posterior lens because the anterior segment is obtained using a lens that is adjacent to the camera of the mobile device and the image of the anterior segment includes a larger area of the anterior segment of the eye than the area of the retina in the image of the posterior segment.

The methods can further include saving the image of the anterior segment if a predetermined quality threshold is met by the one or more quality parameters associated with the image of the anterior segment obtained by the camera of the mobile device. The methods can further include varying an intensity of a variable intensity light source of the lens adapter engaged with the mobile device to illuminate the anterior segment of the eye. The lens can be a macro lens. In some aspects the lens adapter includes: a body, a clamp configured to engage with the mobile device at a first location and a second location, a lens holder engaged with a macro lens movable between a first position in the optical axis of the camera of the mobile device and a second position outside of the optical axis of the camera of the mobile device, an adjustable light source with a light axis parallel to a macro lens optical axis, a third engagement surface configured to slidably engage with the mobile device at a third location, wherein the clamp defines an axis and the body of the anterior adapter portion is configured to move along the axis of the clamp. In some aspects the lens adapter further includes a complementary surface of the body configured to reversibly engage with a base section of a posterior portion, the posterior portion comprising: the base section configured to reversibly engage with the complementary surface of the body of the lens adapter, a telescoping section movable relative to the base section, and a lens holder engaged with a distal end of the telescoping section configured to removably engage with an indirect lens, the base section configured to removably engage with the body of the anterior adapter portion to form an optical axis between the ophthalmoscopy lens and the camera of the mobile device. The methods can further include automatically focusing the camera of the mobile device on the image of the anterior segment of the eye. The methods can further include presenting the image of the anterior segment of the eye that meet the predetermined quality threshold on a display of the mobile device. The methods can further include sending one or more of the images of the anterior segment of the eye that meet the predetermined quality threshold to an electronic medical record (EMR) or electronic health record (EHR) of the patient. The methods can further include automatically saving the one or more of the images of the anterior segment of the eye to the EMR or EHR of the patient. The methods can further include saving the image to a cloud storage network in a HIPAA compliant manner. In some examples the image is encrypted.

The methods can also include receiving a plurality of images of the anterior segment of the eye of a patient with the camera of the mobile device through the lens of the lens adapter engaged with the mobile device. The methods can further include analyzing the plurality of images of the anterior segment of the eye of the patient, applying one or more digital image processing techniques to the plurality of the images of the anterior segment of the eye of the patient, and forming a combined image of the anterior segment based on the plurality of images of the anterior segment of the eye of the patient and the applied one or more digital image processing techniques.

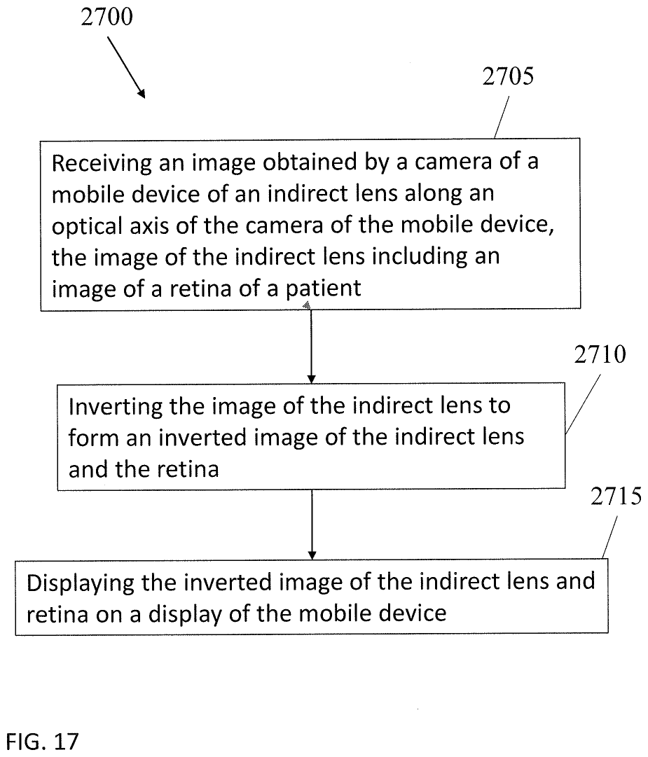

FIG. 17 shows a flow chart of a method 2700 of displaying an image of a retina on a mobile device in accordance with some embodiments. The methods can include receiving an image obtained by a camera of a mobile device of an indirect lens along an optical axis of the camera of the mobile device, the image of the indirect lens including an image of a retina of a patient 2705. The method can include inverting the image of the indirect lens to form an inverted image of the indirect lens and the retina 2710. The method can include displaying the inverted image of the indirect lens and retina on a display of the mobile device 2715. In some embodiments the indirect lens has a size of about 10 D to 90 D. In some aspects the indirect lens is selected from the group consisting of: 14 D, 20 D, 22 D, 28 D, 30 D, 40 D, or 54 D, 60, 66, and 90 D. In some embodiments the indirect lens is removably engaged with a lens mount of a lens adapter. In some aspects the lens adapter is removably engaged with the mobile device. In some embodiments the lens adapter includes a telescoping arm engaged with the lens mount and a base of the lens adapter engaged with the mobile device. The methods can further include varying an intensity of a variable intensity light source of the lens adapter engaged with the mobile device to illuminate the retina. The methods can further include automatically centering the image of the retina on a display of the mobile device. The methods can further include automatically focusing the camera of the mobile device on the image of the retina. The methods can further include presenting the images of the retina that meet a predetermined quality threshold on a display of the mobile device. The methods can further include sending one or more of the images of the retina that meet a predetermined quality threshold to an electronic medical record (EMR) or electronic health record (EHR) of the patient. The methods can further include automatically saving the one or more images of the retina to the EMR or EHR of the patient.

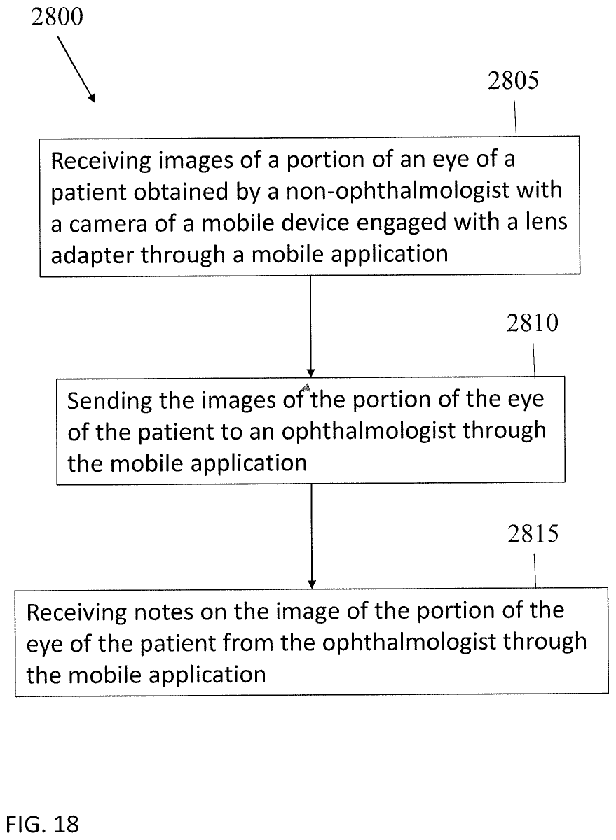

FIG. 18 shows a flow chart of a method 2800 in accordance with some embodiments. The methods can include receiving images of a portion of an eye of a patient obtained by a non-ophthalmologist with a camera of a mobile device engaged with a lens adapter through a mobile application 2805. The methods can include sending the images of the portion of the eye of the patient to an ophthalmologist through the mobile application 2810. The methods can include receiving notes on the image of the portion of the eye of the patient from the ophthalmologist through the mobile application. In some embodiments the ophthalmologist is in a referring network with the non-ophthalmologist. In some embodiments the ophthalmologist is in a referring network of a mobile application database. The methods can further include receiving a referral recommendation from the ophthalmologist for an emergency appointment with an ophthalmologist through the mobile application. The methods can further include receiving a referral recommendation from the ophthalmologist for a non-emergency appointment with an ophthalmologist through the mobile application. The methods can further include receiving an ophthalmology assessment from the ophthalmologist through the mobile application including one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, other eye examination data associated with the patient, and one or more comments from the ophthalmologist. The methods can further include automatically generating a report including the ophthalmology assessment from the ophthalmologist. The methods can further include automatically generating a reimbursement form for the ophthalmologist with billing codes based on the ophthalmology assessment. In some embodiments the image of the portion of the eye of the patient includes an image of a retina of the patient obtained with an indirect lens engaged with the lens adapter. The image of the retina can be obtained using any of the methods described herein. In some embodiments the image of the portion of the eye of the patient includes an image of an anterior segment of the eye of the patient obtained with a macro lens of the lens adapter. The image of the anterior segment can be obtained using any of the methods described herein.

Systems are also provided herein. The systems can include a mobile imaging device with a camera. The mobile imaging device can be configured to run a computer executable code comprising any of the steps of the methods described herein. The systems can also include any of the lens adapters described herein that are configured to removably engage with the mobile imaging device.

In still other aspects, embodiments of a computer readable code executable on a mobile device configured as described above with an external lens is provided that enables a number of alternative methods, steps or ophthalmic examination workflows including one or more steps of capturing, modifying, annotating, sharing, storing and retrieving images of the eye for ophthalmic examination. In some embodiments, the mobile device is adapted to use a lens or lens system as described in commonly assigned co-pending U.S. patent application Ser. No. 15/186,266 entitled “Adapter for Retinal Imaging Using a Hand Held Computer” published as US 2016/0367135, incorporated herein by reference in its entirety. In some aspects, an implementation of the computer readable code executable on a mobile device is adapted and configured for automatic, semi-automatic or user defined operation of a camera module of a mobile computing device alone or in combination with an embodiment of an external lens system as described herein, an alternative external lens system, or other suitable lens system. In some aspects, an implementation of the computer readable code executable on a mobile device is adapted and configured for operation of a camera module of a mobile computing device in combination with an embodiment of an external lens system with an open optical pathway between the mobile device camera module and the patient's eye or an embodiment of an external lens system with a closed optical pathway between the mobile device camera module and the patient's eye.

In some other aspects, embodiments of the computer readable code executable on a mobile device includes a number of image enhancement features. In one aspect, there is provided computer readable code to correct for inverted live image obtained by the camera module of the mobile device. In one implementation, one or more steps of post capture digital image processing will manipulate the manner in which the image is displayed to a user on the screen of the mobile device so that the anatomical features of the eye and as manipulated on the screen are as they would appear if the user were looking directly in the eye as they are on the screen. In other words, the system has the capability of digitally inverting the image of the eye captured by the mobile device so that the images of the eye are presented in an anatomically correct representation on the display visible to the user using the mobile device. In one specific example, one or more digital images of a posterior segment of an eye captured individually or as part of a video stream capture is digitally manipulated so that the optical nerve is oriented so as to be near the patient's nose (nasally) and the macula is oriented so as to be near the patient's ear (temporally), in other words, the optically inverted fundus image is digitally inverted to appear anatomically in the correct orientation. Normally, indirect ophthalmoscopy images are inverted (the image appears upside down).

In some other aspects, embodiments of the computer readable code executable on a mobile device includes one or more options including software implemented options to allow a user or a digital imaging process program on or in communication with mobile device to provide a mask onto a digital image of the eye whereby a selected portion of the image is cropped, covered, blocked or rendered opaque in the image as viewed on the mobile device, stored in memory whether remotely or on the mobile device or shared with another user. In some embodiments, a digital mask may be predefined by a user so that for a particular mobile device image capture of the eye a pre-specified or predefined mask is applied to the captured image. By way of example, a user may predefine an anterior segment mask or a posterior segment mask. In one implementation, the mask is used to remove any extraneous image data captured through the use of a mobile device lens system having an open optical path. The use of a digital mask in this configuration would remove any extraneous image data beyond the eye captured by the mobile device camera module including for example, part of the patient's face, the surroundings of the examination room or furniture and the like, as well as the internal surface of an enclosure in the case of an encased optical pathway. In another exemplary implementation, a digital mask used for image capture of a posterior segment of the eye may direct the user to enlarge the view to fill a predefined ring or viewer or a pre-sized digital mask ring is provided in the screen of the mobile device during an image capture sequence. In still other aspects, an implementation of a digital mask for a mobile device image capture of the eye includes one or more of image recognition software to identify and eliminate known environmental setting objects such as desk, chair, examining room equipment and the like when a typical digital image capture setting has been defined; a predefined, default or preselected digital mask that creates a periscope view about the captured image of the eye so as to eliminate the image surroundings beyond the eye; a user interactive display on the screen to aid in the alignment of the lens attached to the external optical pathway (i.e., an external lens mounted on the mobile device) so that the image of the eye is manipulated by the user so that the eye image corresponds to the lens; user interactive display on the screen to align the eye within the field of view into a preset zone of the lens that is then manipulated by the user or final adjustment; and an image detection program adapted and configured to automatically capture the image in the camera module of the mobile phone when a pre-selected image of the eye is detected in the visual field of the camera unit of the mobile device. In one specifically implemented aspect, an auto image capture program for use in a mobile device camera to obtain an anterior segment of the eye is adapted and configured to detect one or more anatomical structures of the eye, an upper lid, a lower lid, eyelashes, an inner corner of the eye, an outer corner of the eye, an eyebrow, a preselected margin of the skin and structures surrounding the eye. In still other aspects, there is provided a digital imaging program adapted and configured to mask periphery for posterior images of the eye captured using the camera module of a mobile device. In still further aspects, the mobile device is operable with computer readable code having a variety of different camera settings pre-set for the user based on a default value or on a user selected value. Additionally or optionally, the user may then manually adjust the default or preset camera setting value via interaction with the mobile device by touch, voice, motion, pinch, swipe or other configured interaction to indicate the desired modification or change to camera functionality. In one specific implementation, the computer readable code for the mobile phone includes default or pre-set zoom values for the camera unit. In one aspect, the default or pre-set zoom is “zero zoom” when the user indicates or the camera detects an anterior photo is being captured, as well as the optional inclusion of one or more of an adjust zoom, zoom out or zoom in function. In another aspect, the default or pre-set zoom is set to a specific initial zoom setting when the user indicates or the camera detects a posterior photo is being captured, as well as the optional inclusion of one or more of an adjust zoom, zoom out or zoom in function.

In some other aspects, embodiments the computer readable code executable on a mobile device includes one or more options including software implemented options to a user with a visible capture button present on a display visible to the user of the mobile device. In use, capture button enables continuous “slow video” mode, a preset number of single image captures or a video mode. Additionally or optionally, the user may then manually select or indicate or adjust the desired capture mode of the camera module via any interaction with the mobile device by touch, voice, motion, pinch, swipe or other configured interaction detectable by the mobile device and configured to indicate the desired capture mode. In various alternative embodiments one or more of these capture modes is used by the health care provider operating the mobile device to capture digital images of: a patient with limited or impaired ability to maintain gaze; a patient performing steps under commands to look straight ahead, look up, look down, look right and look left; a patient being tested for eye alignment and primary gaze; or capture images of the eye for patients unable to maintain gaze for sufficient length of time to allow immediate examination or image capture. In still other alternative implementations, a camera module of the mobile device operating with computer readable code executable on mobile device is adapted and configured for both still (or burst) and video image capture of patient related information such as a variety of different patient specific image data including, by way of example and not limitation, still or video images captured by the mobile device related to the examination of the posterior aspect of the eye, the anterior aspect of the eye, external presentation of the eye, a patient information card, a patient identification card, a computer screen containing information from a patient medical record, a paper listing or computer screen listing of the patient's prescription listing, a patient intake form, the patient's face, a prior medical history form, or other information obtained from the patient or an electronic record of the patient. Additionally or optionally, a user is provided an interactive review screen of captured images that may be selected for retention or deletion. In the specific case of burst or video mode capture, a user is given the option to “grab” and save the desired or optimal images(s) based on the patient condition or clinical need, and an option to discard the remaining images. In one implementation, the user may use an on screen finger scrolling action to review the captured images and then an on screen finger swiping action to select images for use in the evaluation of a patient condition.

In some other aspects, embodiments of the computer readable code executable on a mobile device includes one or more options including software implemented options for improved mobile device camera module presets camera outputs or operation including zoom and focus based on one or more of: predefined user inputs, default settings corresponding to external lens detected by the system or identified by the user; default settings for digital image capture of the mobile device of the anterior segment of the eye; default settings for digital image capture of the mobile device of the posterior segment of the eye; image orientation correction based on orientation of the mobile device so as to correct orientation of image capture independent of scope position in upright, landscape, or upside down position. In other words, digital camera module of the mobile device will capture still or video camera views and respond accordingly with appropriate host capture processing steps such that the still and video images will be captured, saved, and viewed in the correct orientation. In still further alternatives, computer readable code executable on a mobile device includes instructions that permit a user to designate or select image capture type before image capture.

In some other aspects, embodiments the computer readable code executable on a mobile device includes one or more options including local or remote (i.e., cloud computing) software implemented options for image viewing and sharing of one or more still or video images of a portion of the eye collected by a health care provider. In one aspect, the image viewing and sharing operation is performed on a mobile device adapted and configured according to the computer readable code operated by another health care provider invited to evaluate or consult with the health care provider who used the mobile device to capture still or video images of the eye with the mobile device. In still another aspect, the image viewing and sharing operation is performed on a mobile device adapted by the software executable on a mobile device used by another health care provider being consulted to evaluate the shared one or more still or video images of a portion of the eye and to also include one or more of a comment, evaluation, grade, presence or absence of an lesion, presence or absence of an abnormal finding or any other indication related to an ophthalmic examination of a shared digital image of an eye captured by a mobile device.

In some other aspects, embodiments the computer readable code executable on a mobile device includes one or more options including local or remote (i.e., cloud computing) software implemented options for image viewing digital images of a patient eye directly on a mobile device or on the same device used to capture the digital images of the patient's eye. In one aspect, the digital still and video images of an eye of a patient are organized in the display visible to the user by patient, by a user designated priority or by a user designated tag. In various embodiments, a pre-defined tag or user designated tag may be provided that is related to the presence of one or more disorders of the eye in the digital image of the eye of (i.e., image is tagged after diagnosis). In various embodiments, a pre-defined tag or user designated tag may be provided that is related to adverse findings detected by imaging software or a human operator during an ophthalmic evaluation of the patient's eye. In one aspect, patient captured images are displayed to a user in a manner so that the images are retrieved, organized, or displayed based on presence of one or more tags. It is to be appreciated that tags may be related to a standard ophthalmic examination or a user designated custom tag. In another aspect, a pre-select group of tags is populated by user action on the display based on an existing patient diagnosis or pre-existing condition. In one aspect, the tags are pre-populated for a patient diagnosed with diabetes, or the patient is presented for a diabetic screening.

In still other aspects, embodiments the computer readable code executable on a mobile device includes one or more options including local or remote (i.e., cloud computing) software implemented options for image viewing and annotation of digital images of a patient. In one aspect, the annotation capability is provided by selection of the user sharing an image of a pre-select group of questions or comments to be displayed by the user viewing the shared digital image. In one specific aspect, the tags pre-selected group is populated with pre-defined, user defined, or default questions or comments based on an existing patient diagnosis or pre-existing condition. In another aspect, the user who is viewing the shared image is asked whether the questions or comments are to be viewed or if the image only is to be displayed. In one aspect, the pre-selected information that is shared along with the shared one or more still or video images of the eye includes patient identification information, health information, medication listing or other items of the patient's health condition including any one or more items of information contained in an electronic health record for the patient whose digital images are to be shared. In another aspect, the image sharing and annotation program may also include computer readable instructions on the mobile device allowing the patient to consent in real time with the image share by having the mobile device capture an affirmative interaction with the patient indicate consent either by physical contact with the mobile device, by voice indication or other electronic indication of consent including without limitation a photograph of an appropriate medical consent form signed by the patient.

In still other aspects, embodiments the computer readable code executable on a mobile device includes one or more options including local or remote (i.e., cloud computing) software implemented options for image viewing, sharing only, or sharing with annotation capabilities of digital images of a patient as provided by a secure appropriately configured communication link provided via the mobile device to one or more of an e-mail address from the contacts listing in the mobile device; an e-mail address entered into the mobile device; an e-mail address from a patient medical record (electronic or otherwise); a phone number or a text number. In addition to the above, there are also other implementations that include new message alerts.

In still other aspects, embodiments the computer readable code executable on a mobile device includes one or more options including local or remote (i.e., cloud computing) software implemented options for image viewing using a new encounter view wherein the mobile display is configured to show a view of the latest digital images from all patients aggregated onto one screen.

In still other aspects, there is provided a settings screen on the mobile device display that provides other enhancements to user interaction such as a settings screen to control user preferences for another of the above identified user selected features. In still other implementations of the mobile device imaging system described herein a local database is used on the originating or shared mobile device that may also include an image encryption system or may be adapted and configured to self-delete or automatically delete after a pre-set time period or once the image is viewed.

In still other alternative implementations, embodiments the computer readable code executable on a mobile device includes one or more options including local or remote (i.e., cloud based) software implemented options for pre-selected or user defined ophthalmology workflow. In one aspect, the mobile phone display is adapted and configured to present a number of screens related to an ophthalmic examination of a patient. In still another aspect, the mobile phone display is modified based on user response to a default or user configured ophthalmic examination template. In one aspect, the default or user configured ophthalmic examination template is pre-completed to indicate all findings are normal, and the software implemented method on the mobile display accepts or guides the user to enter or indicate abnormal findings using screen inputs, voice inputs or any other method to indicate an abnormal finding using the mobile phone.

In one aspect, embodiments the computer readable code executable on a mobile device includes an ability for a user to indicate—via interaction with the mobile device—normal findings or abnormal findings of a viewed or displayed digital image of a patient's eye. In one aspect, a normal findings or abnormal findings indicated that the mobile device are related to a portion of the patient's eye, an anterior segment of the patient's eye or a posterior segment of the patient's eye. In still another aspect, embodiments the computer readable code executable on a mobile device include an ability for a user to indicate via mobile device interaction a finding or a diagnosis related to an anterior segment of the patient's eye, a lid, the lashes, the cornea, the conjunctiva, the anterior chamber, the iris and the lens. In still another aspect, embodiments the computer readable code executable on a mobile device include an ability for a user to indicate via mobile device interaction a finding or a diagnosis related to a posterior segment of the patient's eye, the optic nerve, the macula, the vessels or vasculature of the back of the eye, the peripheral regions of the retina and the vitreous chamber. In some additional embodiments, mobile device is adapted and configured to present to the user a preselected or user-defined display for a patient that presents with a pre-existing condition or evaluation of a particular medical condition to be identified by examination of digital images of the patient's eye captured using the mobile device as described herein. In one aspect, the mobile device display is adapted and configured for evaluation of the patient as part of a diabetic screen. In still another aspect, the mobile device display is adapted and configured for evaluation of a patient having diabetes. In still other aspects, the computer readable code operating on the mobile device provides for a user to take photographs of text as part of the use of the mobile device for treatment including functionality such as taking a photograph of patient's medication list, past medical or ocular history, existing medications, as well as functionality to transcribe into text using optical character recognition functionality operable on the mobile device.

While various aspects of the inventive methods of obtaining, sharing and annotating digital still and video images of the patient's eye captured by the camera module of a mobile device have been described in a particular sequence for clarity, it is to be appreciated that implementation of the inventive methods may be adapted and configured to any of a variety of different clinical settings and physician selected operations. For example, the image capturing system described herein may be operated to capture digital images of the patient's eye and in them to storage without annotation or likewise send to another physician for evaluation also without annotation. In another alternative operation, digital images may be captured using the mobile device and immediately annotated with normal and abnormal findings that are stored locally or remotely as part of the patient's record. In still further optional implementation, the user may select only a specific screen or annotation, sharing or storage.

Capabilities of the mobile device digital image capturing and sharing system described herein is capable of a variety of different implementations depending on the level of interaction desired by the user for obtaining information from a third-party reviewer. In one aspect, the user may share an image with a third-party reviewer and then discuss the reviewer's comments orally in real time using any suitable syndication means to connect the parties (i.e., cell phone, land line telephone, internet based communication such as Skype or other voice over IP service). In still another aspect, the user may share an image with a third-party reviewer that includes a predefined or user selected review request specifying specific areas or findings to be reviewed or solicited by the reviewer. In still another aspect, the user may share an image with a third-party reviewer that includes a predefined or user selected review request specifying specific areas or findings to be reviewed as part of a formal consult solicited with the reviewer. In any of the image share protocols described herein may be modified to include along with the still or video images captured by the mobile device: (a) no additional information; (b) a specific review or comment request; (c) a version of the shared image or video stream that may be annotated by the reviewer and (d) information that identifies the patient including without limitation the patient name, medical history, age, sex, past history with disease, current medical listing, or any other information from a patient file or electronic medical record. In one implementation, the mobile device is configured whereby annotation could be done by “drawing” over the image with a finger, a stylus, pop up on screen annotation tool or by other interaction with the image on the mobile device to indicate annotation such as done by “drawing” over the image with a finger or stylus to circle findings as well as adding notes either by typing, voice command or touching with finger or stylus. In a specific implementation of the above methods when patient specific information is offered to a reviewer, the reviewer is provided an option not to view or to limit viewing of the patient specific information (i.e., opt out or partial opt out).

Capabilities of the mobile device digital image capturing and sharing system described herein is capable of a variety of different implementations to enable a remote evaluation by a physician in a remote location. In this exemplary implementation, a technician or health care provider operates the mobile phone image capturing device described above to perform the still or video image capture steps. The captured images are then uploaded, sent or otherwise provided to a physician qualified to evaluate, annotate or otherwise determine the normal or abnormal findings of the digital images. Thereafter, the qualified physician annotates the digital images and saves the annotated images to the patient record, whether locally or in a remote storage system.

In still another alternative embodiment of an eye exam workflow enabled by an embodiment of the present invention, a user provides a secure link to another user to enable that user to view the shared digital images. The user who accesses the images via the secure link is permitted access to the images the secure browser link. Using the secure link user may view, annotate, or otherwise indicate findings or provide information based on reviewing the images so that they may be made available to the user who provided the link. Thereafter, the secure browser is closed and images provided via the link are deleted and no image remains on the mobile device of the reviewer.

In still other aspects, the still or video image data collected from the mobile phone, any annotations provided by a user or a reviewer, any findings provided by a user or a reviewer are adapted and configured using computer readable code executable on a mobile device or via computer operations locally or remotely (i.e., via cloud computers) for storage, use, portability to another mobile or offline platform, evaluation by third party digital imaging analysis systems or software and for any other purpose related to patient care is performed in such a way that the above comply with interoperability requirements such as those for electronic medical records, any ophthalmic API, Fast Health Interoperability Resources (i.e., any proposed interoperability standard developed by the health care IT standards body known as HL7 which is Health Level Seven International (HL7) or any other ANSI-accredited standards developing organization or the Digital Imaging and Communications in Medicine (DICOM) Standard.

In still another alternative embodiment, the above described mobile device based ophthalmic imaging collection, evaluation and sharing system may be modified to include additional features or modules related to mobile phone enabled ophthalmic evaluations. In still another alternative embodiment, the above described mobile device based ophthalmic imaging collection, evaluation and sharing system may be modified to include additional manual input screens, communication links or application programming interfaces (APIs), or mobile platform developed eye test applications to enable use of the mobile device to facilitate, perform or accept results or findings from an ophthalmic testing, evaluation, eye function, drug response, disease progression, or screening, or from a visual acuity test, a bright field test, an assessment of the pupils, a measure of intraocular pressure, a visual field test, an Amsler grid test, a slit lamp evaluation, a funduscopic evaluation or other clinical assessment of the eye and related structures, whether performed manually or with the aid of a computer based testing system, or mobile device enable testing system including any ophthalmic device adapted and configured to be a smart device or a digitally enabled device.

In still another alternative embodiment, the above described mobile device based ophthalmic imaging collection, evaluation and sharing system may be modified to include additional manual input screens, communication links or application programming interfaces (APIs) to enable use of the ophthalmic system described herein—whether operating in the mobile device platform or in a server to server interaction with another system or other modes—to facilitate, perform or accept results or findings, both analog, digital or observed—from an ophthalmic testing, evaluation or screening tool or system. By way of example, if a user has a digitally enabled ophthalmic evaluation or testing device, then the wireless communication capability of that device (such wireless communications capability as Bluetooth or near field or other suitable mode) to transmit the measured reading or test results to the mobile device of the ophthalmic system for recordation in the patient electronic health record as appropriate. As a result, the user of the mobile device based ophthalmic system may acquire patient related data in any number of suitable ways. For example, if a manual device is used, mobile device may provide a data screen to allow the user to enter results manually or by voice or by any other suitable interaction with mobile device. In another example, a device used for the evaluation of a patient's ophthalmic health is adapted and configured to be compatible with and integrated into the workflow associated with mobile device and the software systems described herein. In this case, use of the device would be automatically easily and seamlessly imported directly into the patient record or other indicated site using the mobile device or directly to a remote storage site or other location holding the electronic health record. In still another alternative, a digital ophthalmic device may be linked by a dedicated communication channel or other proprietary data system to allow information collected from the link to device to be imported into the electronic health record or available to the user the mobile device. In still another option, an ophthalmic device is provided that is compatible with the mobile device systems described herein and is provided with an appropriate application program interface that is accepted and configured to operate with the data collection scheme or user interaction defined herein. In summary, the mobile device enabled ophthalmic system described herein may be adapted and configured to receive data inputs either manually or electronically in any of a variety of different forms as described above or as otherwise appropriate to the clinical setting where the data is collected or the availability of directly imported digital patient data.

Adapters are disclosed herein for use with the mobile applications described herein and a hand held computer device to allow a physician, medical professional, nurse, technician, or any user to take an image of a retina of a patient or user. The adapter can engage with the hand held computer device such that a camera on the hand held computer device can line up with an optical axis of the adapter to take a high quality image of the retina. The adjustability of the adapter can allow for the use of the adapter with a variety of different hand held computer devices having cameras located at different areas of the hand held computer devices. Examples of hand held computer devices that can be used with the adapters disclosed herein include tablet computers (iPad®, galaxy note, iPod®, etc.), smartphone devices (Apple@iPhone@, Motorola devices, Samsung devices, HTC devices, etc.), mobile imaging devices, or other electronic devices with a camera.

The light sources on hand held computer devices are typically too bright to illuminate the patient's eye without causing discomfort to the patient. The adapters disclosed herein can include an adjustable light source as part of the anterior adapter. The adjustable light source can easily be adjusted to provide the desired level of light to illuminate the eye of the patient. Another advantage of the inclusion of an adjustable light source on board the adapter is the improvement of the regulatory approval of the device in the U.S. An adapter that uses the light source of the camera of the hand held computer device can require separate regulatory approval for each different model of hand held computer device to show that the light source is safe for use with the eye. The inclusion of the adjustable light source eliminates variability between the light sources for different hand held computer devices and streamlines the regulatory approval process in the U.S.

WO 2014/194182 discloses a modular lens adapter system for anterior and posterior segment ophthalmoscopy with separate adapters for the anterior imaging and posterior imaging. Lining up the optical axis of the posterior ophthalmoscopy lens, the light source, and the camera can provide some challenges in the field and make the device more difficult to use. The present disclosure discovered that combining the anterior segment adapter and the posterior segment adapter greatly simplified the use of the device by eliminating additional steps to line up the optical axes of the different pieces of the system. The fixed relationship between the optical axis of the anterior adapter portion and the optical axis of the ophthalmoscopy lens greatly simplifies the ease of use of the adapter system and can improve image quality.

The adapter systems described herein can be used to obtain images of the eye of the patient that are comparable to the images obtained using expensive equipment typically only found in doctor's offices. The images obtained using the adapter systems described herein can be used for treatment, diagnosis, and triage purposes.

The portability, ease of use, rugged construction, and low cost enable the adapter systems described herein to be used with a hand held computer to obtain images of the patient's eyes at the doctor's office and outside of the doctor's office. For example, the systems can be used inside and outside in locations lacking a doctor's office or other healthcare provider. The suitability of the adapters for outdoor use allows for a healthcare provider to travel to remote locations to treat patients that lack access to healthcare facilities. The adapter systems can also be used by a general practitioner to send to an ophthalmologist for diagnosis and referral based on the absence or presence of a medical problem with the eye visible in the captured images.

The adapter systems can be configured to removably engage with a hand held computer device with a camera having an optical axis. The adapter systems can include an anterior adapter portion and a posterior portion. The anterior adapter portion can include a body, a clamp configured to removably engage with the hand held computer device, a lens holder, an adjustable light source, a third engagement surface configured to slidably engage with the hand held computer device, and a complementary surface on the body configured to reversibly engage with a portion of the posterior portion.

The clamp can be configured to contact the hand held computer device at a first and second location. In some embodiments the first and second location are on opposing surfaces of the hand held computer device. The clamp can define an axis and allow for the body of the anterior adapter portion to move along the axis of the clamp to line up the optical axis of the camera with the optical axis of the lens in the lens holder.

The lens holder can be adapted to support a macro lens. The lens holder can include a hinge such that the lens holder can move between a first position in the optical axis of the camera and a second position outside of the optical axis of the camera. In some embodiments the macro lens can have a circular dominant cross-section. In other embodiments the macro lens has a dominant plane orthogonal to the optical axis of the macro lens with a non-circular cross-sectional profile. The macro lens can have the non-circular cross section with a portion of the lens removed to adjust the engagement between the macro lens/lens holder and a surface of the body of the anterior adapter portion.

In some embodiments a plurality of the modules described herein, such as the beam splitter module, slit beam module, blue filter, different sized apertures, etc. can be removably engaged with the anterior adapter portion. In some embodiments one or more of the modules can engage with the anterior adapter with a hinge or through a plurality of hinged parts, like in a Swiss army knife. The modules can swing into place and be used and then moved out of the way of the optical path or light source path. For example, the modules could be used in the order of direct ophthalmoscopy with the beam splitter module, followed by the slit beam module, followed by the blue light filter. The modules can be attached along a hinge with a common axis like in a Swiss army knife type configuration. In other cases the modules can each be attached at a different hinge that is adapted to move the module into and out of the desired position (e.g. in the optical pathway or light pathway). For example some modules could engage with the hinge 141. Other modules could engage with a hinge on the back side of the anterior adapter portion to cover the optical pathway or light source. In other embodiments the modules can be removably attached and interchangeable in place of one another, for example the modules can engage with a common section of the anterior adapter. Examples of engagement types include magnets, reversible engagement through complementary mating surfaces, snap on or friction fits, etc.

The adjustable light source can have a light axis parallel to an optical axis of the macro lens or other lens in the lens holder and/or an optical axis of the camera of the hand held computer. In some embodiments the light axis of the adjustable light source can be perpendicular or orthogonal to the optical axis of the camera.

The third engagement surface can be configured to slidably engage with the hand held computer device at a third location. The third engagement can secure the anterior portion relative to the hand held computer device after the optical axis of the camera and the anterior adapter portion have been lined up.

The posterior portion can include a base section configured to reversibly engage with the complementary surface of the body of the anterior adapter portion, a telescoping section movable relative to the base section, and a lens holder engaged with a distal end of the telescoping section configured to removably engage with an ophthalmoscopy lens. The base section can be configured to removably engage with the body of the anterior adapter portion to form an optical axis between the ophthalmoscopy lens and the camera of the hand held computer device.

The lens holder can be engaged with an ophthalmoscopy lens. When the system is not in use the ophthalmoscopy lens can be removed from the lens holder. The ophthalmoscopy lens can be configured for indirect ophthalmoscopy. The lens mount can be sized to accommodate an ophthalmoscopy lens in the range of 10 D to 90 D, such as a 14 D, 20 D, 22 D, 28 D, 30 D, 40 D, or 54 D, 60, 66, and 90 D condensing lens for indirect ophthalmoscopy. The working distance between the lens mount and the hand held computer device can be about 5.75″ in the case of an iPhone and a Volk Panretinal 2.2 lens, but will vary depending on the combination of hand held computer device camera, ophthalmoscopy lens power, and the subject being examined. For instance, for certain combinations of patients and lenses, the working distance can be reduced approximately 2 inches, or lengthened to approximately 10 inches. Ophthalmoscopy lenses can be easily mounted and removed from the inner diameter of the lens holder.

The lens holder can be engaged with a lens holder hinge that is engaged with the telescoping section of the posterior portion. The lens holder hinge can provide for movement of the lens holder between a first position in the optical axis of the camera and a second position outside of the optical axis of the camera. The second position can include a position where the lens holder is folded flush with the telescoping section.

The clamp and third engagement structures of the anterior adapter portion allow for the optical axis of the anterior adapter to be moved along the x-axis 154 and y-axis 156 relative to the hand held computer device. The optical axis of the anterior adapter can be adjusted to line up with the optical axis of the camera of the hand held computer device.

The clamp includes a first surface configured to engage with the first location of the hand held computer device and a second surface configured to engage with the second location of the hand held computer device. The first surface and second surface can include a rubber surface or other surface to increase friction and prevent relative movement between the first and second surfaces and the hand held computer device. The first surface and second surface can be on opposing sides of the hand held computer device. In some embodiments the clamp is spring loaded. In some embodiments the clamp is configured to apply a compressive force to the first and second location.

The third engagement surface for the hand held computer device can include a hook or semi-circular shape. In some embodiments the third engagement surface has a semi-circular or hook shape configured to slidably engage with the hand held computer device at the third location. The third engagement surface can be adapted to hold a surface of the body of the anterior adapter against a surface of the hand held computer device. Different sized third engagement surfaces can be used to accommodate hand held computer devices with different camera locations.

In some embodiments the third engagement structure is configured to removably engage with the anterior adapter portion. The adapter system can include a plurality of different third engagement structures that can be have different geometries. The third engagement structure with the desired geometry can be selected based on the location of the camera on the hand held computer device and the dimensions of the hand held computer device.

In some embodiments the third engagement structure can include an adjustable engagement mechanism configured to engage with the hand held computer device. The adjustable mechanism can assist with securing the third engagement structure relative to the hand held computer device and can help accommodate hand held computer devices of varying thickness. In some embodiments the adjustable engagement mechanism can include a thumb screw and a hand held computer engagement surface with the thumb screw being adjusted to provide a compressive force on the hand held computer device with the engagement surface. In some embodiments the adjustable engagement mechanism can include a spring, a hand held computer engagement surface, and a release lever. The spring can provide a compressive force on the hand held computer device and the release lever can be used to quickly disengage the adjustable engagement mechanism.

Once the anterior adapter portion has been positioned to line up the optical axis with the optical axis of the camera the adjustable positions can be secured with a plurality of locking mechanisms to prevent or limit further relative movement between the hand held computer device and adapter.

The adapters can include an anterior locking mechanism on the anterior adapter portion configured to position the anterior body relative to the axis of the clamp. The anterior locking mechanism can be adapted to secure a length of the axis of the clamp such as by securing the first surface of the clamp relative to the second surface of the clamp. The anterior locking mechanism can also secure the body relative to the first surface and second surface of the clamp. In some embodiments the anterior locking mechanism is a thumb screw mechanism.

The posterior portion can also include a locking mechanism to secure the telescoping section relative to the base section of the posterior portion. In some embodiments a thumb screw locking mechanism can be used to secure the telescoping section. In other embodiments a friction fit can be used between the telescoping section and the base section. In some embodiments the telescoping section can move with a twisting motion similar to the structures used in SLR camera lenses.

The posterior portion can also include a lens holder locking mechanism configured to secure the lens holder relative to an axis of the telescoping section. For example the lens holder can be secured when the lens holder engages with an ophthalmoscopy lens to hold the ophthalmoscopy lens in the optical axis of the camera. The lens holder can also be secured when in a folded configuration flush with the telescoping section. The lens holder locking mechanism can include a thumb screw mechanism.

The flashes used on many hand held computer devices are often too bright for most patient eyes, and/or they are too variable in their characteristics from device to device to be reliably or safely used at the discretion of a user. The adjustable light source on the anterior adapter portion provides a softer amount of light to the eye of the patient so that high quality images can be obtained while minimizing or eliminating patient discomfort from the light source. The use of an the adjustable light source on the anterior adapter portion with a softer amount of light made it easier to comply with regulatory authorities to show the amount of light provided to the eye was safe. Yet another benefit of the adjustable light source on the anterior portion is that it eliminates variability between the light sources on different hand held computer devices. The use of an adjustable light source on the anterior adapter portion also streamlined the regulatory review process for the device because the same adjustable light source of the anterior adapter portion is used with any of the hand held computer devices. As a result the adjustable light source could be reviewed for safety once with the anterior adapter portion subsequently approved for use with any hand held computer device versus regulatory review and approval for each light source on each hand held computer device to be used with the adapter.

The adjustable light source is integral with the body of the anterior adapter and powered by a power source within the anterior adapter. In some embodiments the light source comprises a light-emitting diode (LED). In some embodiments a light diffuser can be used with the adjustable light source. In some embodiments the anterior adapter portion includes a light source control configured to adjust the properties of the light source. In one example the light source control is a dial. In other examples the light source control is a slider or a set of buttons, e.g. a plus and minus button to increase or decrease the intensity. The anterior adapter can include a battery compartment within the body of the anterior adapter portion to power the adjustable light source.

In some cases an open optical pathway between the lens holder and the camera can be used when imaging the retina. This configuration can be used in lower light environments, such as those that can be present indoors or in a doctor's office or healthcare provider office.

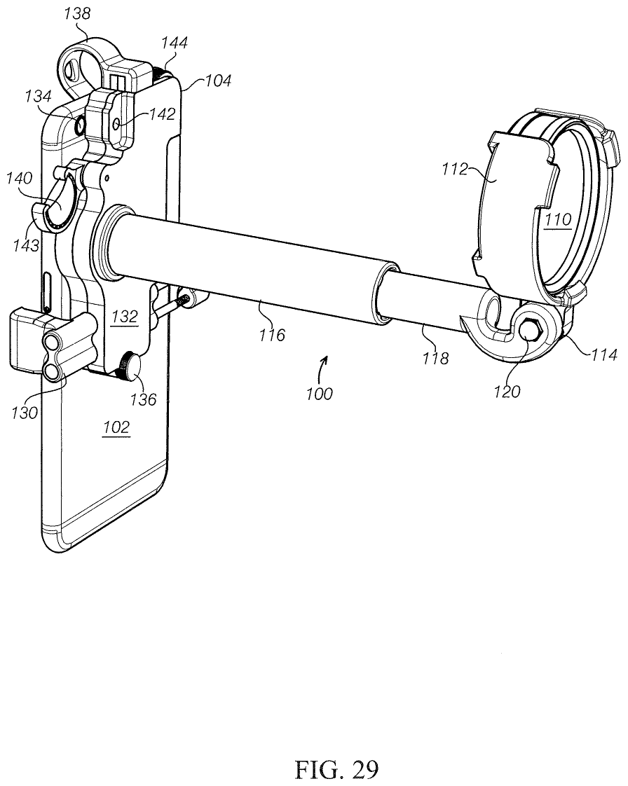

In some cases, such as outdoor settings where examinations can be performed in poorer countries and remote settings away from healthcare facilities, a cover can be used to block exterior light along the optical pathway between the camera and the ophthalmoscopy lens and posterior lens holder. Reducing or blocking the exterior light can improve the image quality and brightness of images of the patient's eyes. In some embodiments a removable cover configured to removably engage with the posterior portion is used to form an enclosure to reduce and block light from the optical pathway. The removable cover can include a clamping mechanism to engage with the posterior portion, such as the telescoping section. The removable cover can also include a telescoping portion configured to adjust a length of the removable cover to match the length of the telescoping section. For example, when the telescoping section is adjusted to improve the image of the retina in the ophthalmoscopy lens the cover length can move with the movement of the telescoping section. The removable cover can include a proximal portion with an opening to accommodate the camera of the hand held computer device and the light source of the anterior adapter portion and a distal section to engage with the lens holder. The distal section of the cover can include a groove or opening to engage with the lens holder hinge to receive all or a portion of the lens holder within an internal volume of the cover. The telescoping can be accomplished through a twisting or sliding mechanism. In some embodiments telescoping can be automated through the use of a wirelessly controlled motor. In some embodiments a second lens can also be positioned within the enclosure to create a compound lens optical pathway. In some embodiments the enclosure portion itself can telescope and a separate telescoping section is not used. For example the telescoping enclosure can direct engage with the anterior adapter portion as shown in FIGS. 29A and 29B.

The adapter systems can be combined with modular units to obtain additional images of the eye. For example, a beam splitter module can be used for direct ophthalmoscopy of the eye. A slit lamp module can be used to obtain optical cross-sectional images of the cornea and anterior chamber of the eye.

In some embodiments a beam splitter module is provided for use with the adapters disclosed herein. The beam splitter module can be configured to removably engage with the anterior adapter. The beam splitter module, when engaged with the anterior adapter, is configured to direct light from the adjustable light source to be coaxial with the optical axis of the camera. The beam splitter can include one or more mirrors to reflect light from the adjustable light source to be coaxial with the optical axis of the camera. The beam splitter can also include a polarizing light filter in the optical pathway of the adjustable light source when the beam splitter module is engaged with the anterior adapter portion. The beam splitter can also include a polarizing light filter in the optical pathway of the camera when the beam splitter module is engaged with the anterior adapter portion. A polarizing light filter can also be placed over the LED light source as well as in combination with the polarizing light filter over the camera lens, or used alone over the LED.

In some embodiments a slit beam module configured to removably engage with the anterior adapter can be used with the adapter. The slit beam module can be engaged with the anterior adapters described herein to provide for some of the functionality of a conventional slit lamp device. The slit beam module creates a rectangular beam of light using a spherocylindrical lens, a rectangular aperture, or both. The slit beam module either approaches the eye at a fixed angle relative to the optical pathway, or with an adjustable angle. The aspect ratio of the rectangular beam is also optionally adjustable to a size of 0.5 mm×0.5 mm, to a longer aspect ratio such as 15 mm×0.5 mm to 14 mm×5 mm, or as large as 15 mm×15 mm out to diffuse lighting such that there can be little or no perceivable borders.

In some embodiments the systems can include a light shaping module configured to be removably engaged with the anterior adapter portion to modify the adjustable light source. The light shaping module includes a plurality of light shaping structures. In one example the light shaping module can include one or more of: a first aperture, a second aperture that is larger than the first aperture, a convex lens, a plano-convex lens, a spherocylindrical lens, a slit lamp, and a blue filter.

In some embodiments the base section includes a magnet to engage with the anterior adapter portion. The anterior adapter portion can include a complementary magnet to engage with and line up the posterior portion such that the posterior portion has the desired orientation relative to the optical pathway of the anterior adapter portion. In some embodiments the magnets can be used in addition to separate complementary engagement surfaces, such as a groove and male counterpart to the groove.

In some embodiments the telescoping section has a closed optical pathway. The closed optical pathway can include a built in ophthalmoscopy lens.

Methods are also provided for using the adapters described herein to capture images of the anterior segment and posterior segment of the eye of a patient. For example the anterior adapter portion can be engaged with and lined up with the optical axis of the camera of the hand held computer device. The macro lens and lens holder can be moved to a position in the optical axis of the camera. Next, the hand held computer device and adapter can be positioned to capture an image of the anterior segment of the eye of the patient using the camera, adjustable light source, and the macro lens. After the macro lens has been used the macro lens holder can be moved to a position outside of the optical axis of the camera. For imaging the retina, the posterior portion can be engaged with and secured relative to the anterior adapter portion. An ophthalmoscopy lens is engaged with the lens holder. Next, the length of the telescoping section can be adjusted to properly focus the ophthalmoscopy lens on the desired portion of the eye of the patient. The adjustable light source can also be adjusted to provide the desired illumination to the eye of the patient. An image of the retina of the patient can then be captured with the camera and the ophthalmoscopy lens. The posterior adapter is typically used on a patient with a dilated pupil (e.g. through the use of a topical mydriatic agent).

For bright outdoor or bright indoor settings the removable cover can be used. The removable cover can be engaged with the posterior portion followed by adjusting the length of the telescoping section and adjustable light source to obtain an image of the patient's eye through the ophthalmoscopy lens.

For direct ophthalmoscopy the beam splitter module adapter can be engaged with the anterior adapter portion. The beam splitter can be engaged with the adjustable light source to reflect the light emitted from the adjustable light source to be coincidental with the optical axis of the camera of the hand held computer device. The optical axis of the camera can be used to direct the path of the light source through the pupil of the eye of the patient without dilation (e.g. non-mydriatic) to obtain an image of the retina of the patient via direct ophthalmoscopy.

Examples of a hand held slit lamps along with methods for using such a hand held slit lamps are disclosed in U.S. Pat. No. 4,461,551, the disclosure of which is incorporated by reference in its entirety herein.

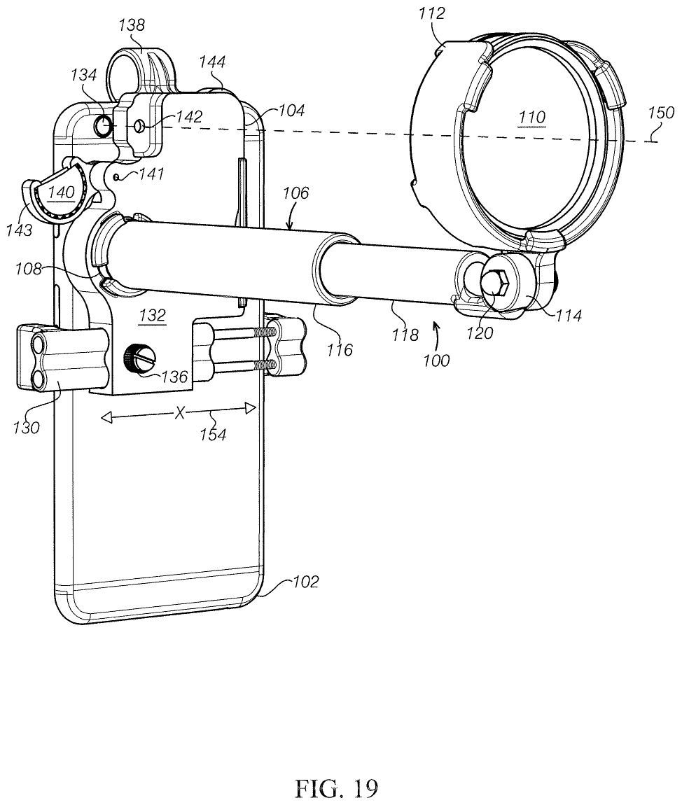

FIG. 19 is a front view of an adapter 100 attached to a hand held computer device 102 in accordance with some embodiments. The adapter 100 includes an anterior adapter portion 104 and a posterior portion 106. The posterior portion 106 can be configured to removably engage with the anterior adapter portion 104 at a base 108. The posterior portion 106 includes a lens 110 (such as an ophthalmoscopy lens) and lens holder 112. The posterior portion 106 can include a base shaft 116 and telescoping shaft 118 configured to move relative to one another to modify the length of the posterior portion 106. The lens holder 112 can be connected to the telescoping shaft 118 at an adjustable hinge 114. The hinge 114 can be secured with an adjustable locking screw 120. The adjustable screw 120 can also be configured to lock the movement of the telescoping shaft 118 relative to the base shaft 116 in some embodiments.

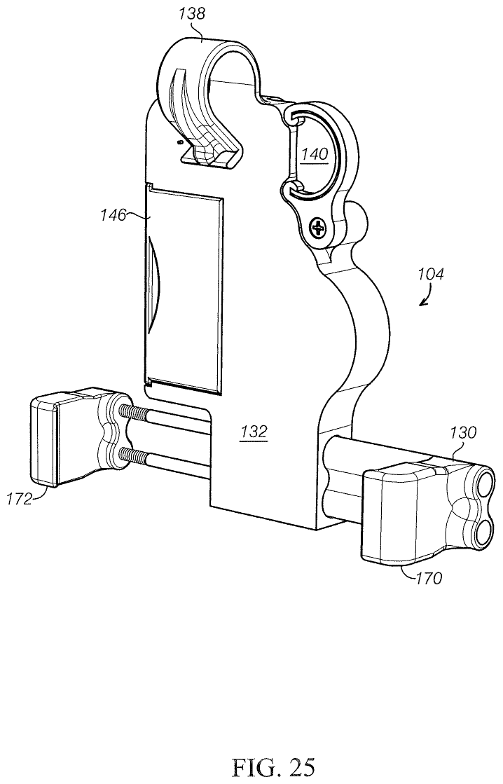

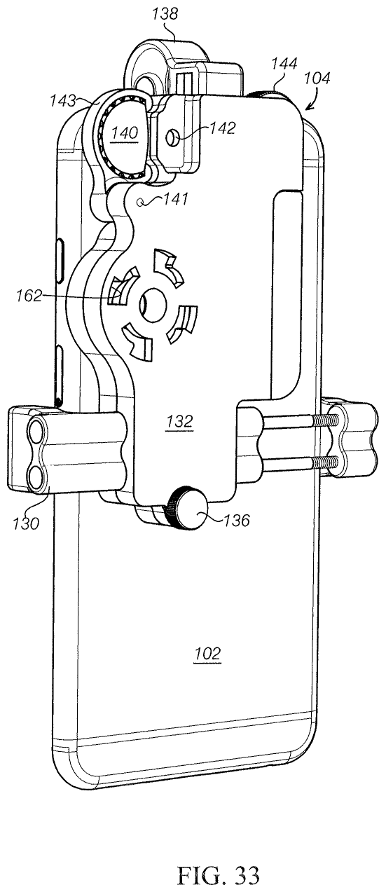

The anterior adapter portion 104 can be configured to receive the base shaft 116 at base 108, such as with the complementary mating surface 162 shown in FIG. 33. The anterior adapter portion 104 can be configured to engage with the hand held computer device at multiple contact points. For example, the illustrated adapter 100 engages the hand held computer device at three contact points. The adapter 100 can be configured to be movable relative to the hand held computer device along a vertical y-axis 156 and horizontal x-axis 154. The illustrated adapter 100 includes an adjustable horizontal clamp 130 configured to allow the anterior adapter portion body 132 to move horizontally (along the x-axis 154) to align the optical axis 150 of the camera 134 of the hand held computer device 102 with the optical axis of the adapter 100. The anterior adapter portion body 132 can be secured relative to the horizontal clamp 130 by a locking mechanism 136, such as the illustrated adjustable screw. The illustrated adapter 100 includes a third engagement surface or vertical contact point 138, illustrated with a hook type configuration to hold the hand held computer device 100 flush with the anterior adapter portion 104. The a third engagement surface 138 can hold the hand computer device 100 flush with the anterior adapter portion 104 while still allowing the anterior adapter portion body 132 to move or slide horizontally relative to the adjustable horizontal clamp 130. The dimensions and length of the third engagement surface 138 can be modified to accommodate different hand held computer device locations (see FIGS. 25A and 25B). For example, a longer hook could be used to accommodate a hand held computer device with a camera closer to the middle of the y-axis 156 of the hand held computer device. The adjustable horizontal clamp 130 can be spring loaded or use another mechanism to securely contact the hand held computer device 100. The adjustable grip can be configured to securely engage the hand held computer device edges by applying a compressive force between the two contact points where the adjustable horizontal grip engages with the hand held computer device. The adjustable grip can be sized to accommodate hand held computer devices having various widths.

The adjustable horizontal clamp 130 can allow the macro lens 140 and light source 142 to be aligned with optical axis 150 of the hand held computer camera 134. Different hand held computer devices have different dimensions and different cameras positions. For example, the iPhone 6 is in the left corner, many android phones are centrally located and further away from the edge, HTC phones are located in the right corner, etc. The anterior body can be adjusted relative to the adjustable horizontal clamp 130 to align the camera 134 with the lenses 110, 140.

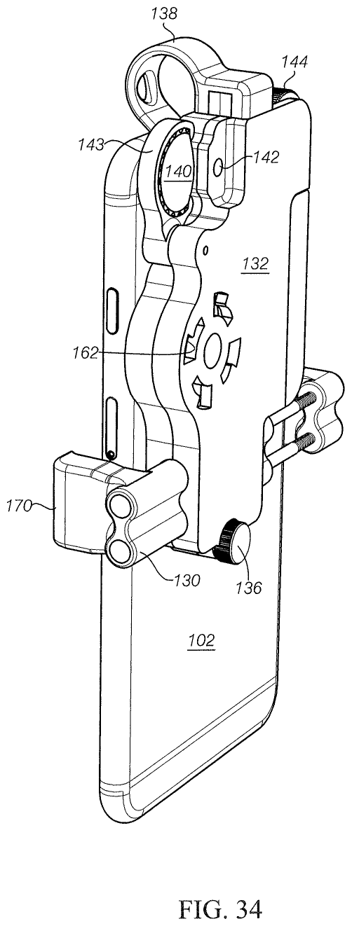

The illustrated anterior adapter portion 104 also includes a macro lens 140, macro lens holder 143, and lens holder hinge 141, light source 142, and light source dial control 144. The illustrated light source 142 is a LED. The lens holder 143 can be adapted to receive other types of lenses. The lens 140 and lens holder 143 can rotate about the lens holder hinge 141 to move the macro lens 140 between a position in the optical axis 150 of the camera and a second position outside of the optical axis of the camera 150. FIG. 19 shows the macro lens 140 and lens holder 143 at a position outside of the optical axis 150 of the camera. FIG. 34 shows the macro lens 140 in the optical axis of the camera 150. The light source 142 can be controlled by the light source control 144, which is illustrated as a rotatable knob or dial. The light source 142 can also include one or more optional light diffuser elements. The optional light diffuser elements can be within the housing and in front of the light source 142.

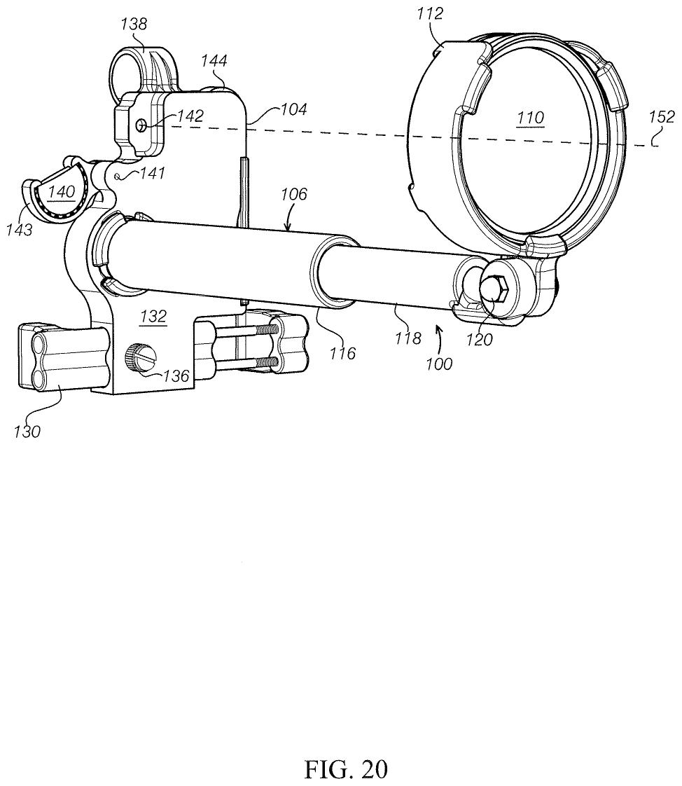

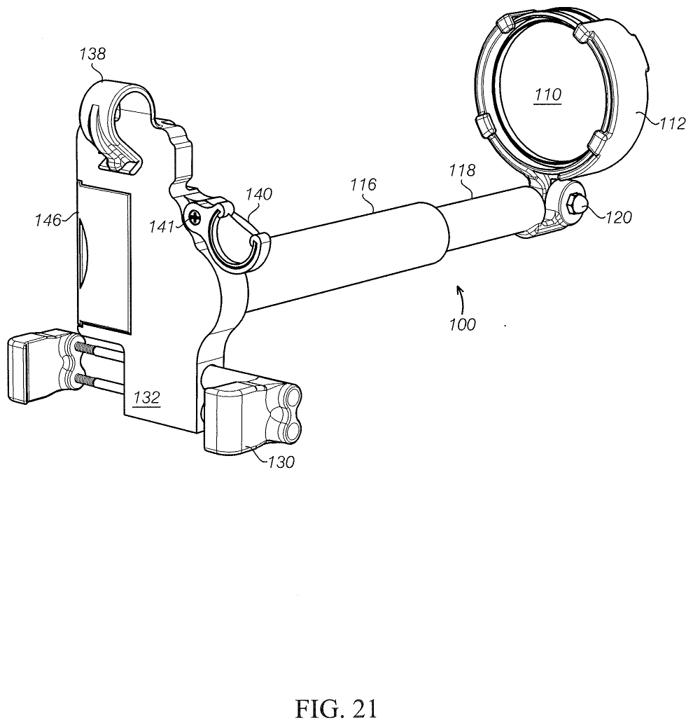

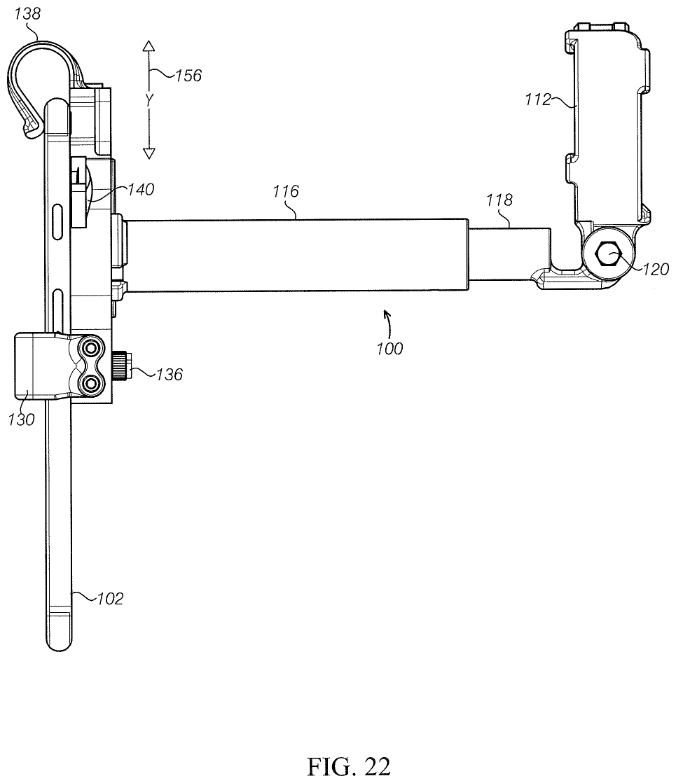

FIG. 20 is a front view and FIG. 21 is a back view of the adapter 100 of FIG. 19 without a hand held computer device 102. The adjustable light source 142 has an optical axis or pathway 152. The anterior adapter portion body 132 includes a battery compartment 146 configured to receive a power source, such as a battery. FIG. 22 is a side view of an adapter in accordance with some embodiments.

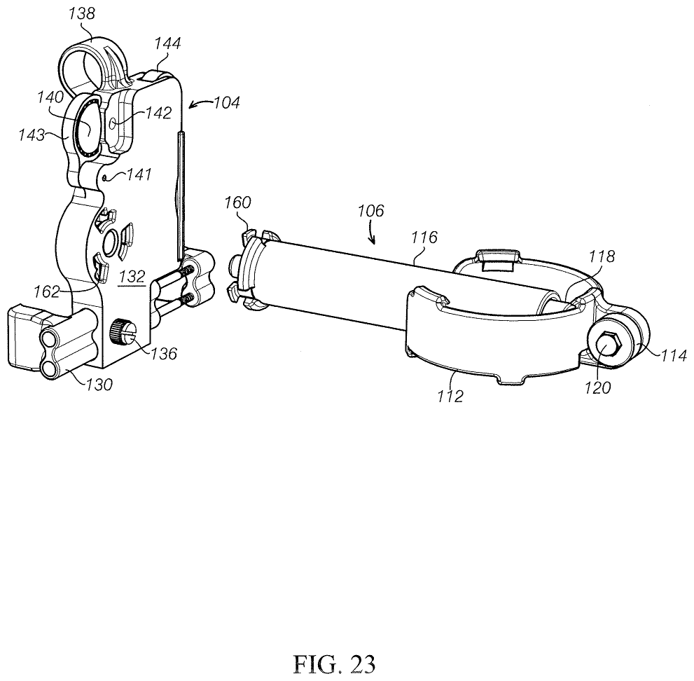

FIG. 23 illustrates the anterior adapter portion 104 and posterior portion 106 of the adapter 100 separate from one another. The posterior portion 106 is illustrated with the lens holder 112 in a folded position relative to the telescoping section 118. The posterior portion 106 includes a male engagement structure 160 configured to be received within a complementary mating structure 162 on the anterior adapter portion body 132. The illustrated engagement structures 160, 162 are configured to lock in place by turning the surfaces relative to one another. The ability to disengage the posterior portion 106 from the anterior adapter portion 104 can improve the portability and storage of the device while also decreasing the likelihood of the posterior portion being damaged. The adjustable screw 120 can be adjusted to fold the lens holder 112 as shown in FIG. 23. The adjustable screw 120 can also be adjusted to retract the telescoping shaft 118 relative to the base shaft 116 as shown in FIG. 23.

The axial length between the camera 134 and the lens 110 can be adjusted by moving the telescoping shaft 118 relative to the base shaft 116 to achieve the desired distance. The axial length can be adjusted until the camera 134 can record a desired image of the retina. The horizontal position along the x-axis 154 of the anterior adapter portion body 132 to line the optical axis 150 of the camera 134 with the lens 110.

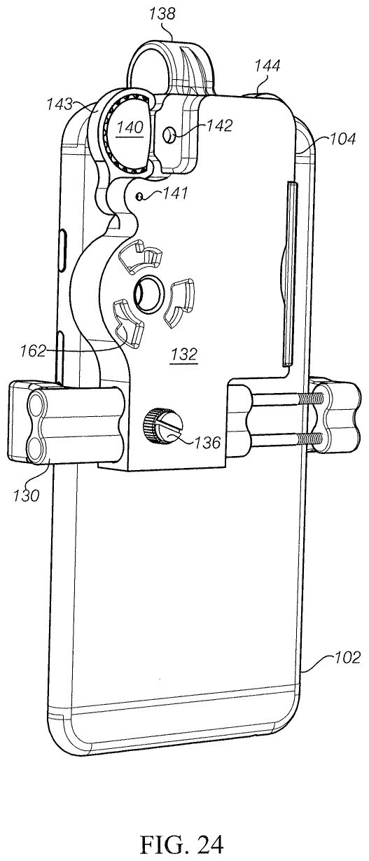

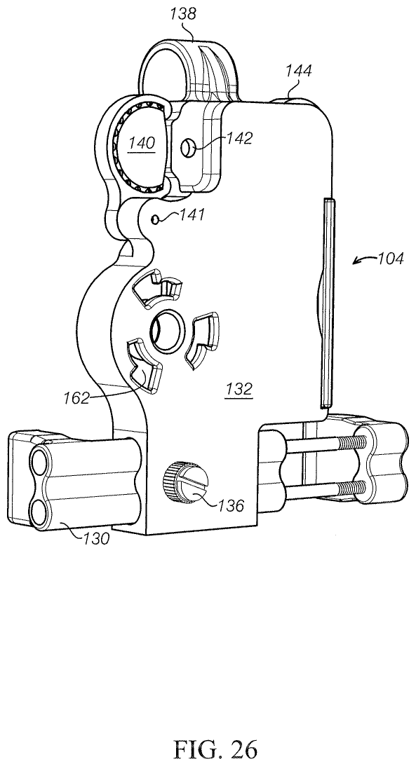

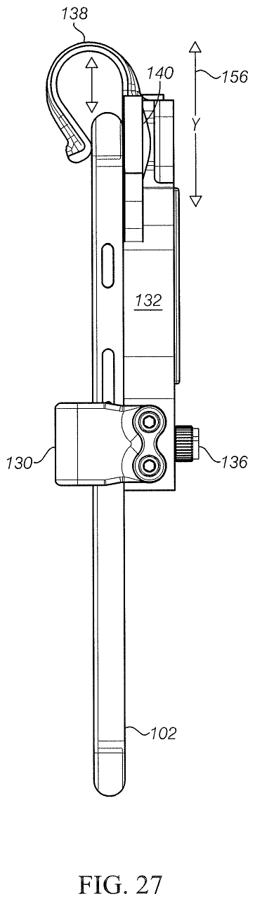

FIGS. 24-27 illustrate various views of the anterior adapter portion 104 of the adapter 100. The adapter 100 can be securely held to the hand held computer device 102 by the three-point connection between the anterior adapter portion 104 and the hand held computer device 102. The adjustable horizontal clamp 130 can be spring loaded to securely clamp on to the hand held computer device 102 with the first clamp surface 170 and second clamp surface 172. Moving the anterior adapter portion body 132 relative the adjustable horizontal clamp 130 allows for the optimal positioning of the lens 140 and light source 142 relative to the camera 134. FIG. 27 shows how the third engagement surface 138 can move along the y-axis 156 to accommodate different hand held computer device camera locations.

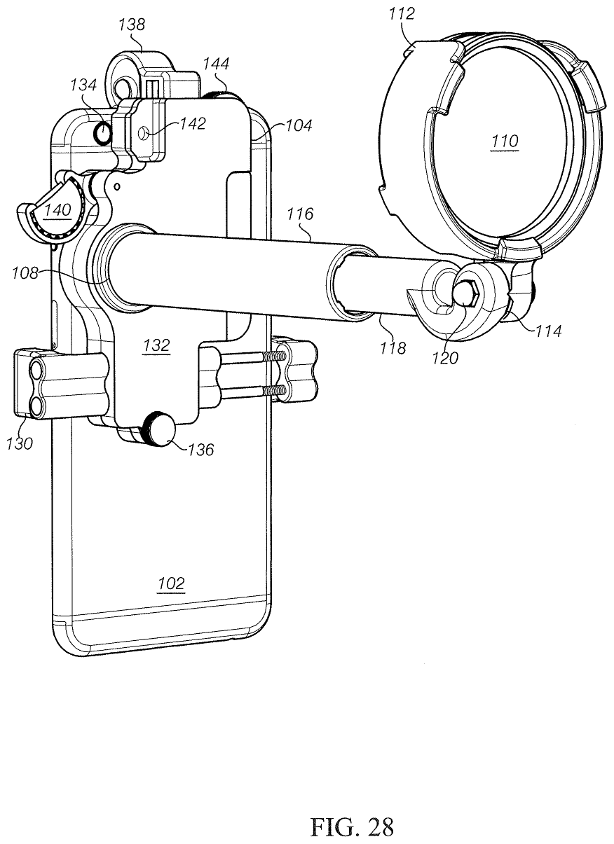

FIGS. 28-29 illustrate front views of the adapter attached to the hand held computer device with the macro lens 140 out of the optical axis 150 of the camera 134. The length of the telescoping section is shorter in FIGS. 28-29 versus the configuration illustrated in FIG. 19.

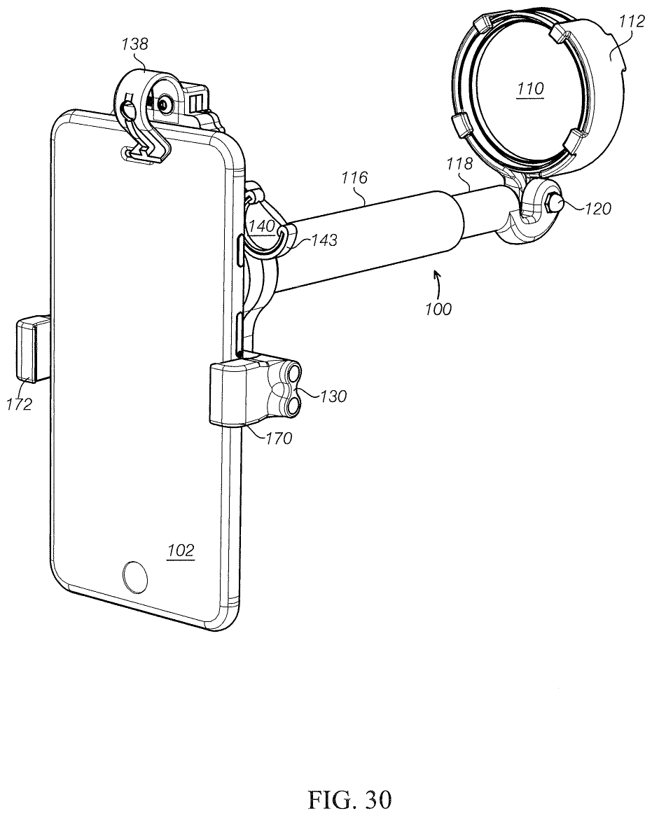

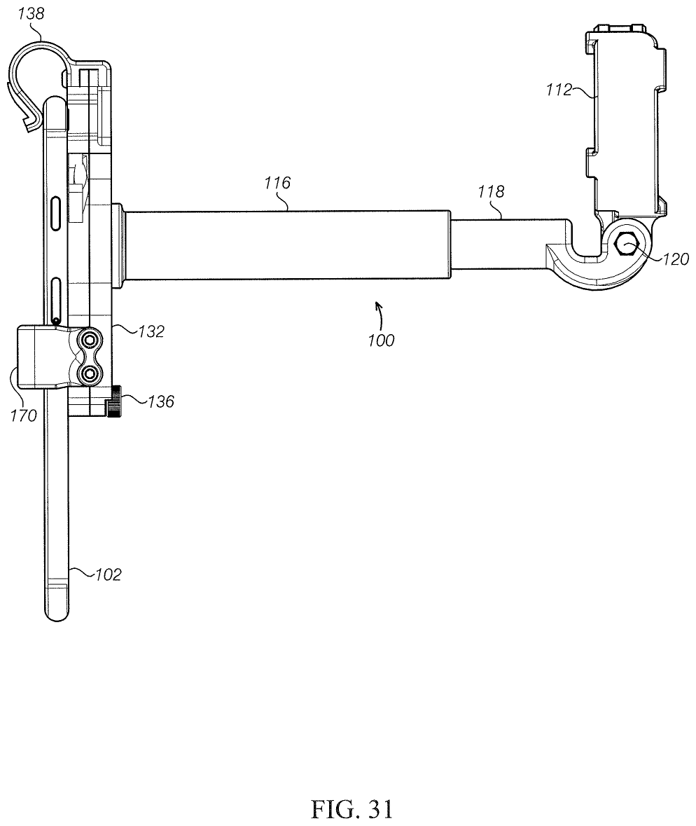

FIG. 30 is a back view of an adapter attached to a hand held computer device 102 in accordance with some embodiments. The display side of the hand held computer device 102 is shown in FIG. 30. FIG. 31 is a side view of an adapter 100.

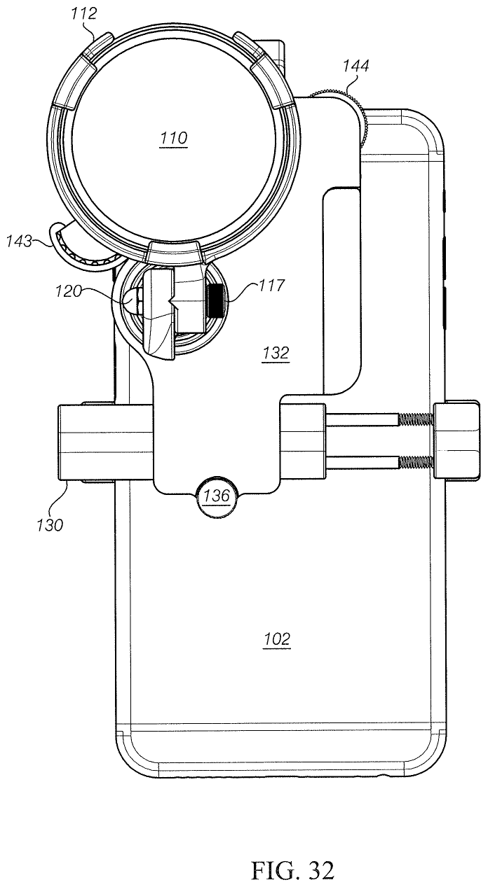

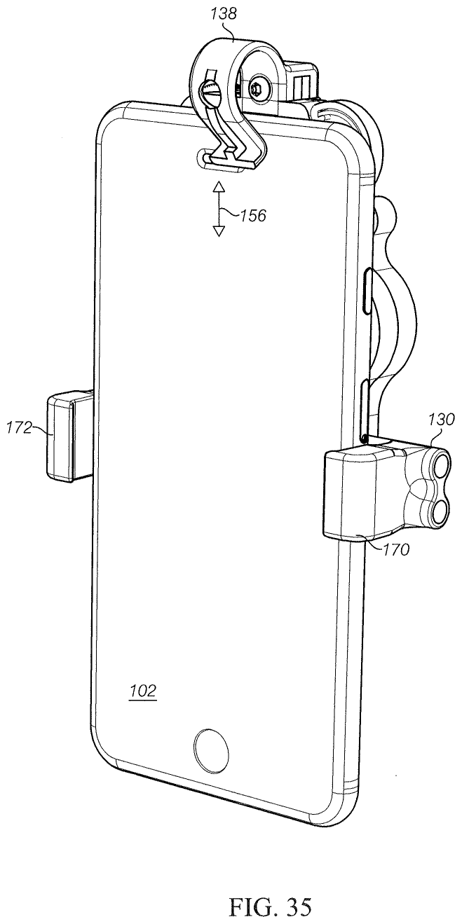

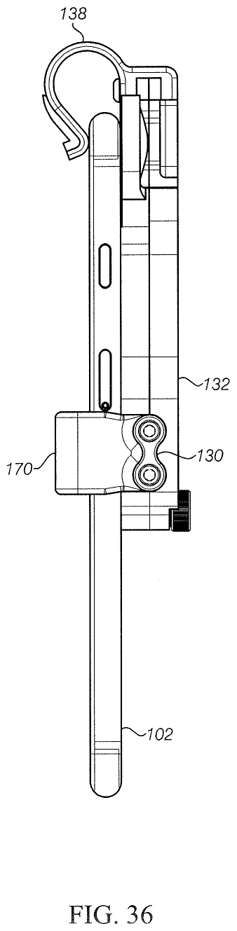

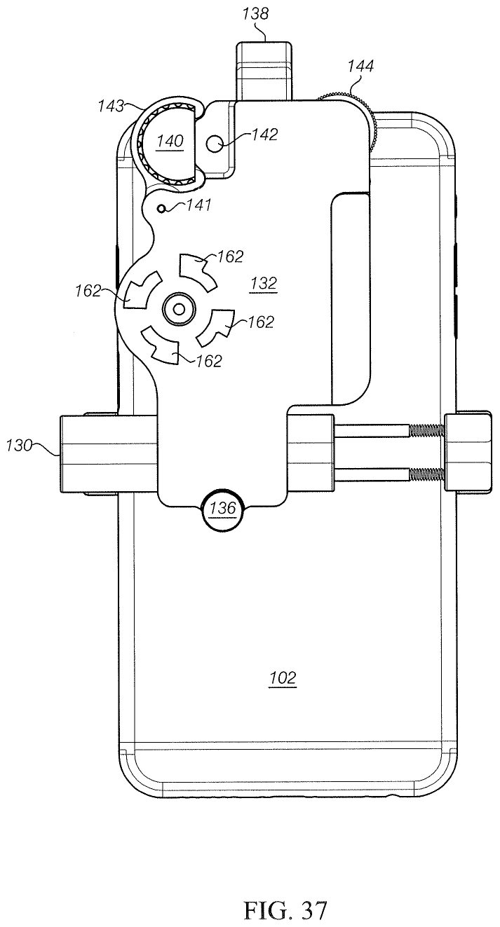

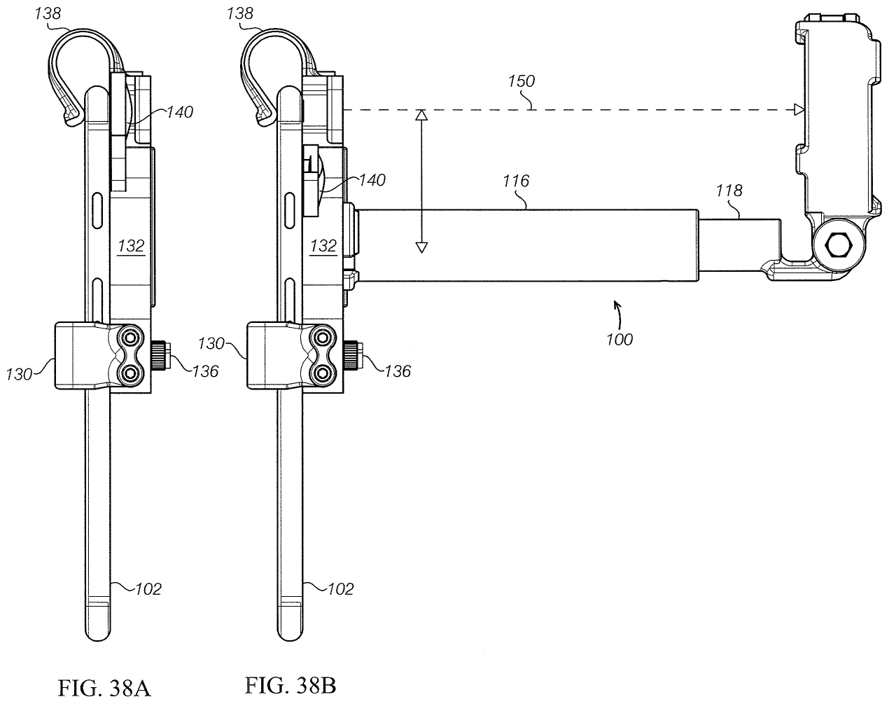

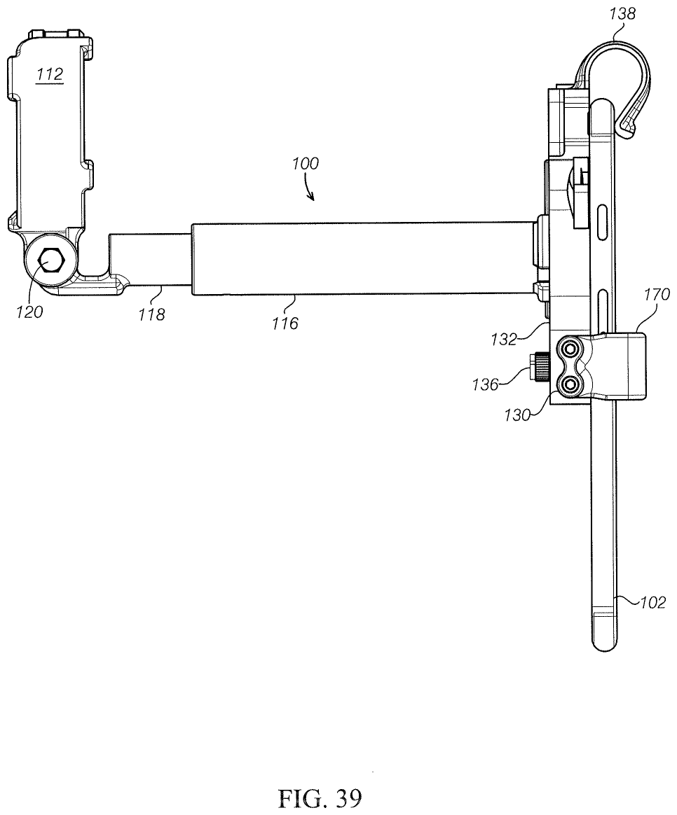

FIG. 32 is a front view of an adapter attached to a hand held computer device 102 in accordance with some embodiments. FIG. 32 shows the telescoping section locking mechanism 117 that can be used to secure the relative movement between the base section 116 and telescoping section 118 of the posterior portion 106. The dial 144 is adapted to adjust and control the intensity of the light source. FIGS. 33-39 illustrate additional views of the adapter 100.

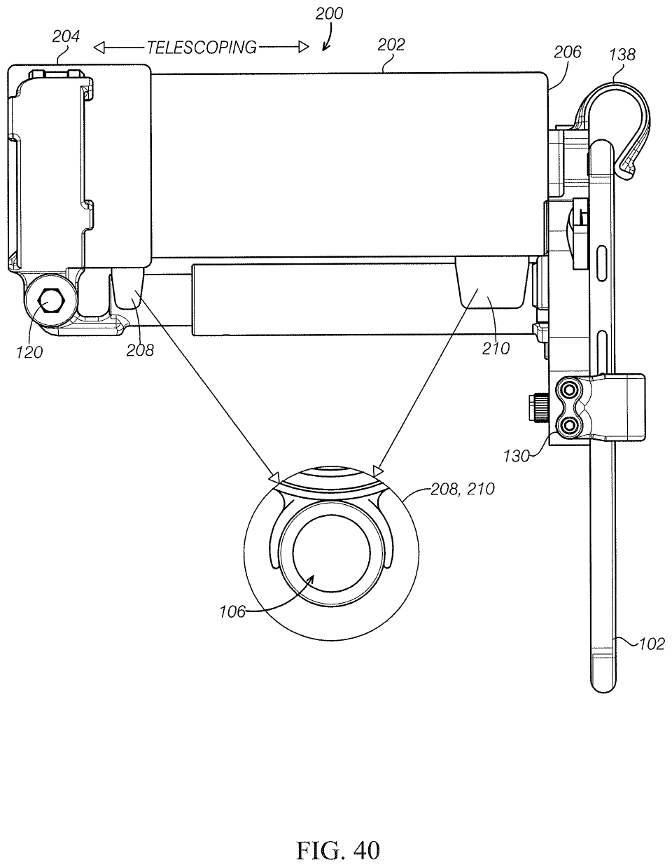

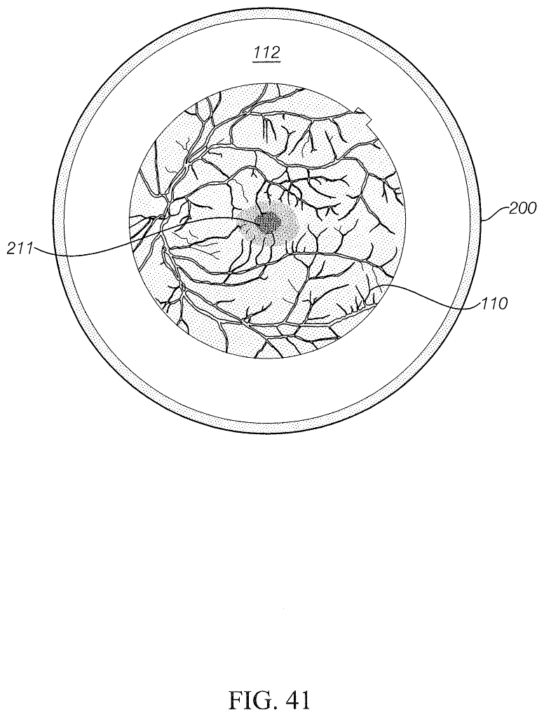

FIG. 40 illustrates a side view of an adapter engaged with a hand held computer device 102 and optional, reversibly attached optical pathway enclosure 200 in accordance with some embodiments. The enclosure adapter 200 includes a first portion 202 and second portion 204 configured to move relative to one another to move with the telescoping section of the posterior portion. The enclosure adapter 200 includes a first clamp 208 and second clamp 210 configured to engage with the telescoping portion and base portion of the adapter. The enclosure adapter 200 includes a back portion 206 configured to engage with the camera 134 of the hand held computer device. The enclosure adapter 200 can block out exterior light to improve the quality of the images captured using the posterior portion. FIG. 41 illustrates an exemplary cross-sectional view that can be produced by the adapters described herein. The cross-sectional view shows the enclosure adapter 200, ophthalmoscopy lens 110, lens holder 112, and retina 211. An image of the retina 211 can be captured by the camera 134.

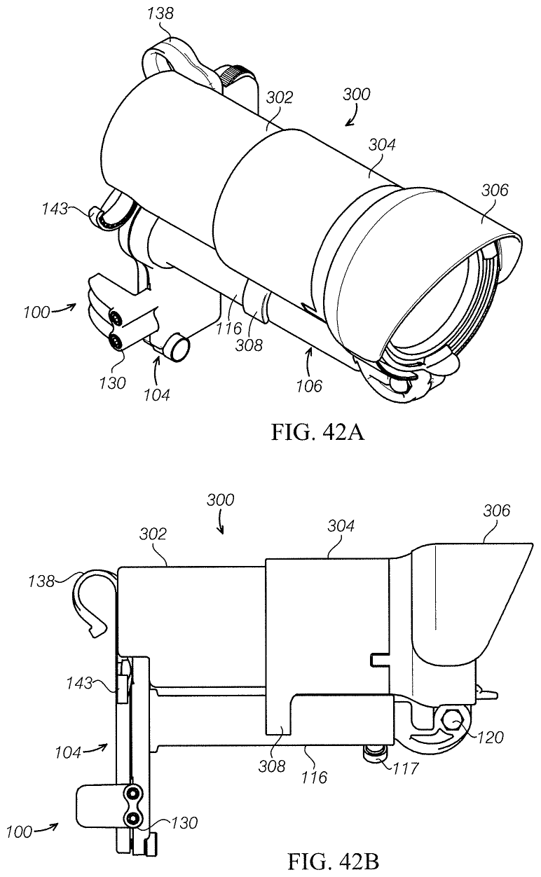

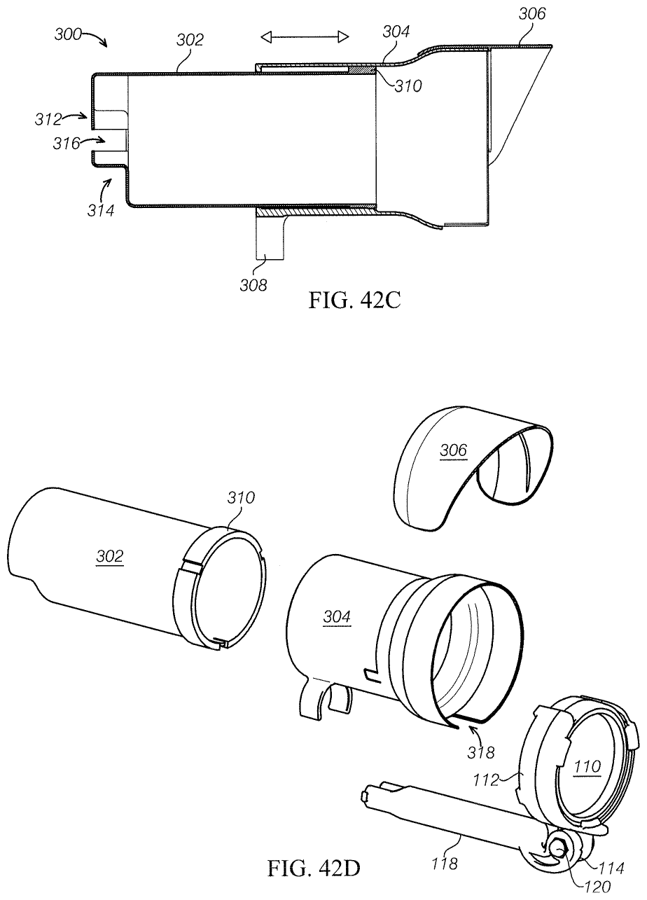

FIGS. 42A and 42B illustrate views of an optical pathway enclosure adapter 300 engaged with an adapter 100 in accordance with some embodiments. FIGS. 42C and 42D are cross-sectional and exploded views of an optical pathway enclosure adapter 300. The enclosure adapter 300 is configured for blocking exterior light from the optical pathway between the ophthalmoscopy lens 110 and the camera 134 of the hand held computer device. The enclosure adapter 300 includes a first portion 302 and second portion 304. An optional third portion 306 can be used to provide additional blocking of exterior light from the ophthalmoscopy lens 110. The enclosure adapter 300 includes a clip 308 for removably engaging with the telescoping section 118 and/or base section 116. The first portion 302 and second portion 304 can slide relative to one another so that the length of the first portion 302 and second portion 304 can be adjusted to match the length of the poster portion 106. The first portion 302 includes a stop 310 to limit axial movement between the first portion 302 and second portion 304. The first portion includes a back cover portion 312 with a hand held computer engagement surface 314 and an opening to accommodate the light source 142 and camera 134 of the hand held computer device. The second portion 304 includes a groove 318 to engage with and receive a portion of the lens holder 112 to hold the lens 110 within the second portion 304 of the enclosure 300. FIGS. 42A-42B illustrate the macro lens 140 and lens holder 143 out of the optical axis of the camera 134.

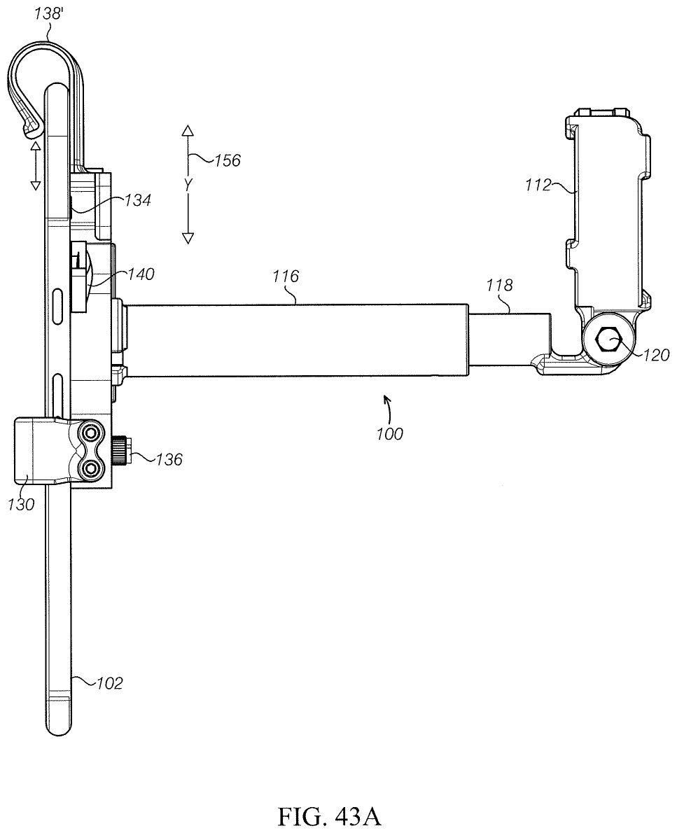

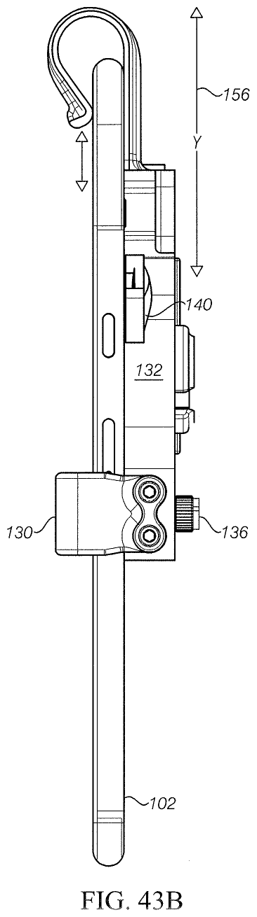

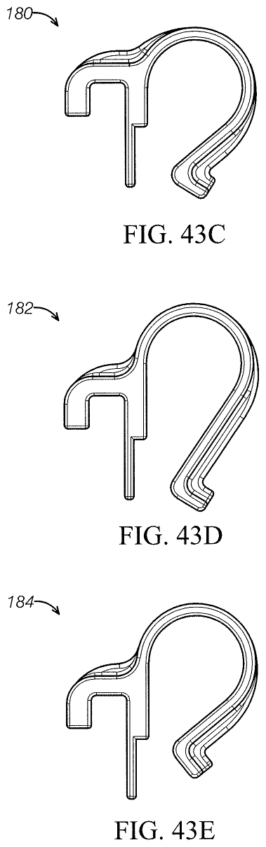

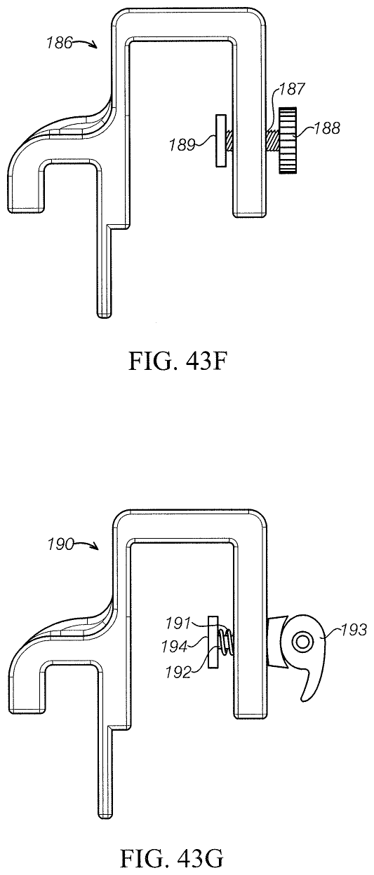

FIGS. 43A and 43B illustrate additional embodiments of an anterior adapter 100 with alternate configuration for the third engagement structure. The illustrate third engagement structures 138′ have different lengths to accommodate movement of the adapter relative to hand held computer device along the y-axis 156 to line up the optical axis of the camera with the optical axis of the macro-lens 140 or ophthalmoscopy lens 110. The adapters 100 can be provided with multiple sizes of third engagement structures 138/138′ so that the end user can removably engage the third engagement structure 138/138′ having the appropriate geometry based on the camera location of the hand held computer device. FIGS. 43C-43E illustrate third engagement structures 180, 182, and 184, respectively, with varying geometry. The adapters described herein can include multiple geometries of third engagement structures that can be removably engaged with the anterior adapter 104 based on the geometry and location of the camera 134 of the hand held computer device 102.

FIG. 43F illustrates a third engagement structure 186 with an adjustable engagement structure including a screw 187, knob 188, and soft padding 189 for engaging the hand held computer device 102. FIG. 43G illustrates a third engagement structure 190 with an adjustable engagement structure including a spring 191, quick release shaft 192, quick release lever 193, and padding 194 for engaging the hand held computer device 102. In some embodiments the adjustable third engagement structures 186, 190 shown in FIGS. 43F-43G can be used instead of the clamp 130 and third engagement structure 138 used in other embodiments. Thus, in this alternate configuration a single contact point can be used to secure the anterior adapter portion 104 to the hand held computer device 102.

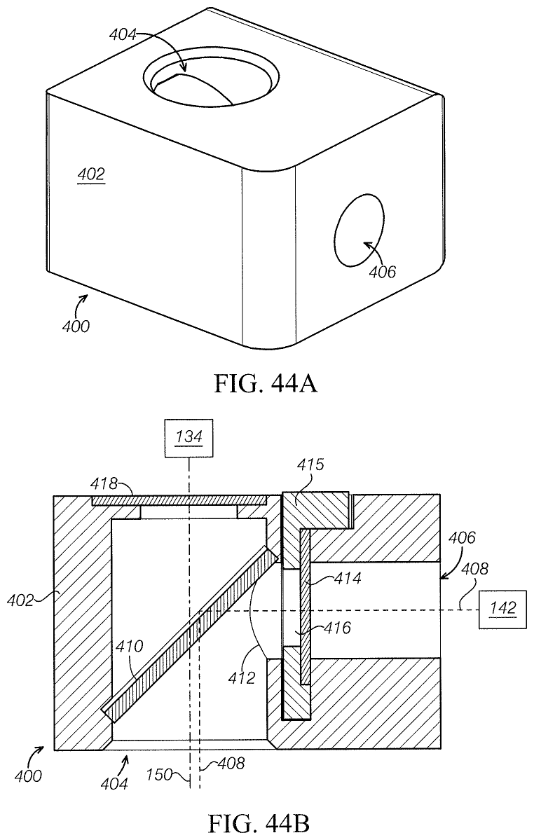

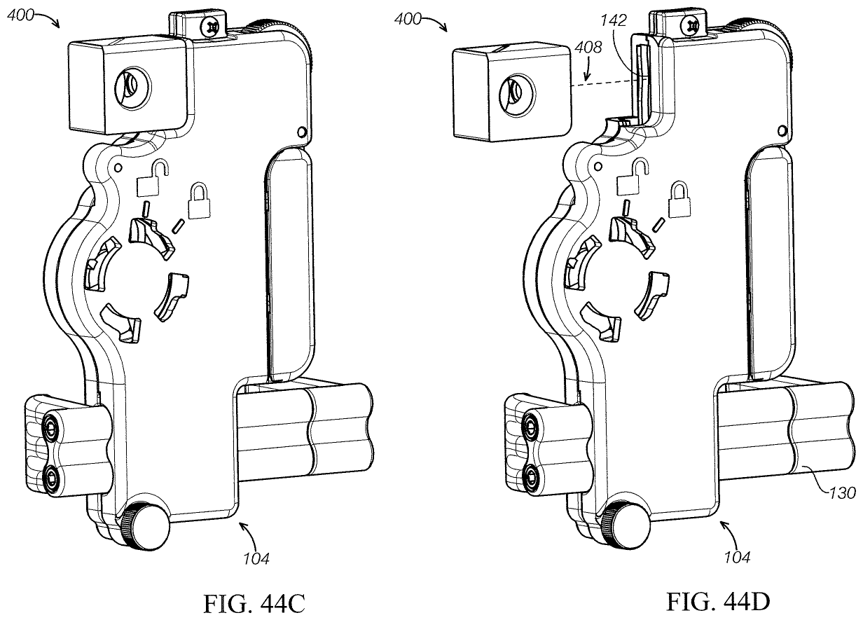

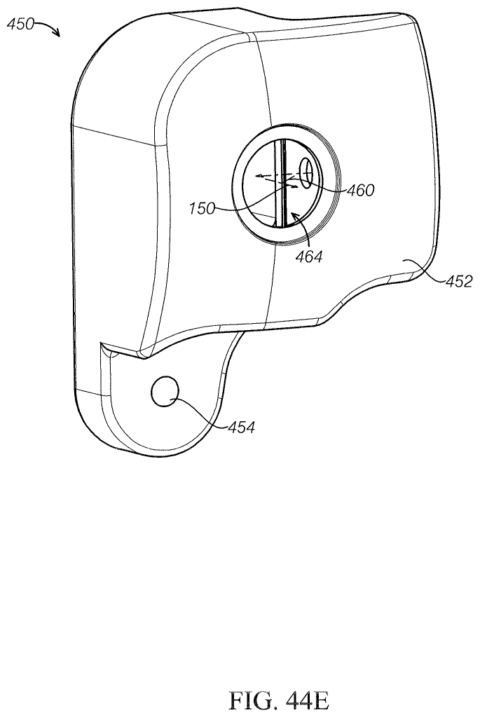

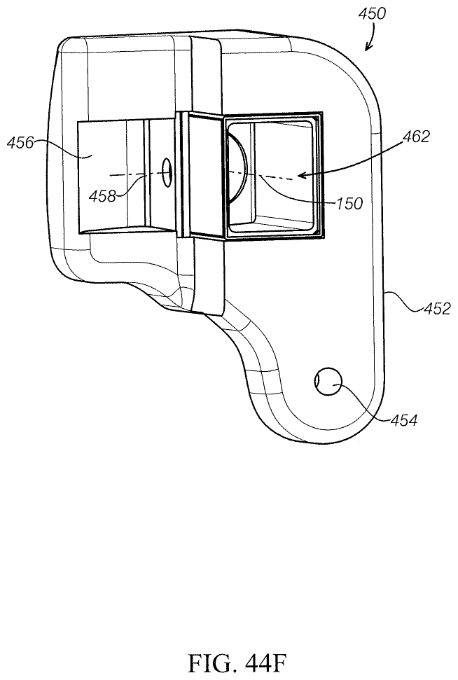

FIGS. 44A and 44B illustrate an exterior view and cross-sectional view, respectively, of a removable beam splitter module 400 in accordance with some embodiments. FIGS. 44C and 44D illustrate the beam splitter module 400 separate from and engaged with an anterior adapter 104, respectively, in accordance with some embodiments. The beam splitter module 400 includes an exterior housing 402, opening 404, and light source opening 406. Light emitted from the adjustable light source 142 enters the beam splitter module 400 along light path 408 through light source opening 406 and is reflected off of mirror 410 to be coaxial with the optical axis 150 of the camera 134. The beam splitter module 400 can also include a polarizing filter 414, polarizing holder 415, and pinhole 416 along the light path 408. The beam splitter can also include an optional lens 412 to further modify the light path 408 of the light emitted from the adjustable light source 142. In one example the optional lens 412 can condense the light into a circular shape. The beam splitter module 400 can also include a polarizing filter 418 adjacent to the camera 134. The anterior adapter 104 illustrated in FIGS. 44C and 44D has a light source 142 that emits light in the direction of the dominant axis of the clamp 130. In this embodiment, the light source within the anterior adapter is oriented such the light is emitted laterally into the side of the beam splitter module 400. The beam splitter module 400 allows the anterior adapter 104 to capture images with the camera 134 through a pupil of the eye that is not dilated thereby enabling direct ophthalmoscopy of the retina of the patient.

FIGS. 44E and 44F illustrate another embodiment of a beam splitter module 450 that is adapted to receive light from the light source 142 orthogonally to the body 132 of the anterior adapter 104. The removable beam splitter module 450 includes 452 and a hinge or pivot 454 that can in some embodiments removably engage with the hinge 141. The removable beam splitter module 450 can rotate about the hinge or pivot 454 to position the removable beam splitter module 450 adjacent to the adjustable light source 142 or out of the optical path of the light source. The removable beam splitter module 450 includes a first mirror 456 that reflects the light along pathway 458 towards the second mirror 460. After the light reflects off of the second mirror 460 the light path 458 is coaxially with the optical pathway 150 of the camera 134 of the hand held computer device 102. The removable beam splitter includes an opening 460 for the light path 458 to exit the module such that the light path 458 to be coaxial with the optical pathway 150 of the camera 134 of the hand held computer device. The removable beam splitter includes an opening 462 adapted to be positioned adjacent to the camera 134.

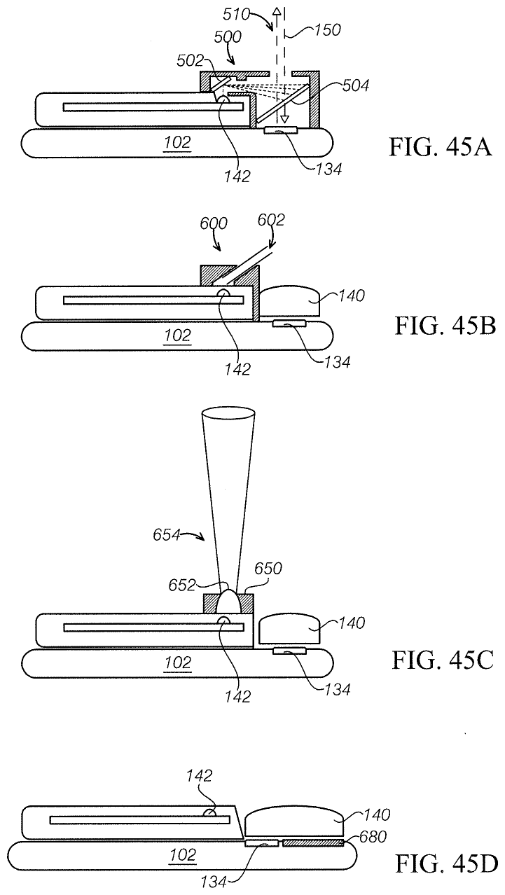

FIG. 45A illustrates an anterior adapter engaged with an embodiment of a beam splitter module 500. The beam splitter module 500 includes a first mirror 502 and second mirror 504. The beam splitter module 500 can removably engage with the anterior adapter such that the light source 142 of the anterior adapter portion is directed along pathway 510 in line with the optical axis 150 of the camera 134 of the hand held computer device 102. The beam splitter module 500 can include optional polarizing filters along the optical pathway of the light source 142 and/or optical pathway of the camera 134.

FIG. 45B illustrates an anterior adapter engaged with an embodiment of a slit beam module 600 including a slit lamp 602 to direct the light diagonally from the light source 142 of the anterior adapter.

FIG. 45C illustrates an anterior adapter engaged with an embodiment of a light beam collimation or condensation module 650. The collimation module 650 can removably engage with the anterior adapter. The collimation module 650 includes a light collimating element 652 that directs the light from the light source 142 to focus the light along light path 654.

FIG. 45D illustrates an anterior adapter engaged with an embodiment of a mask module 680. The mask module 680 can assist users in lining up the camera 134 with the macro lens 140 and optical pathway of the adapter. The mask module 680 is an extension of the anterior adapter portion that includes a small aperture through which the user aligns the camera 134.

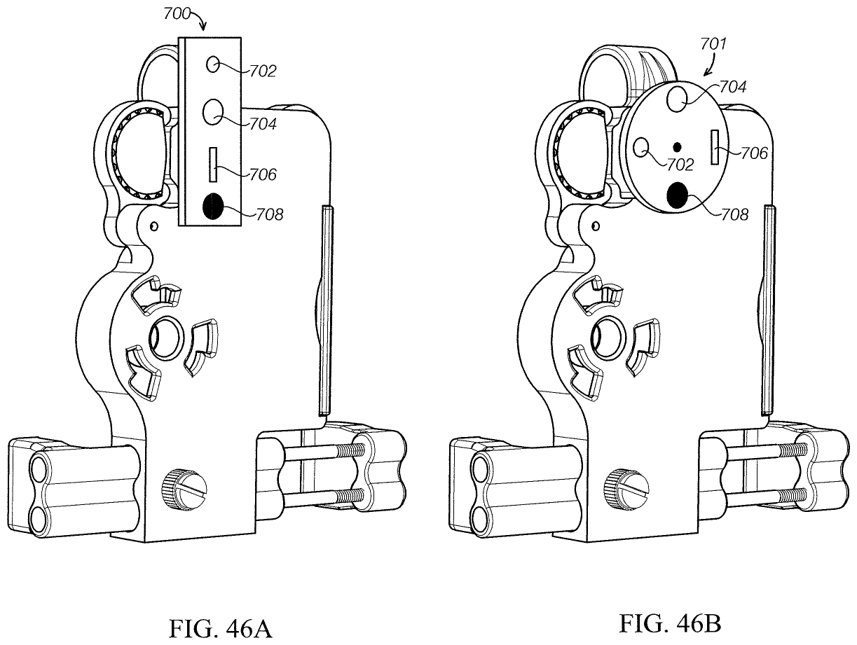

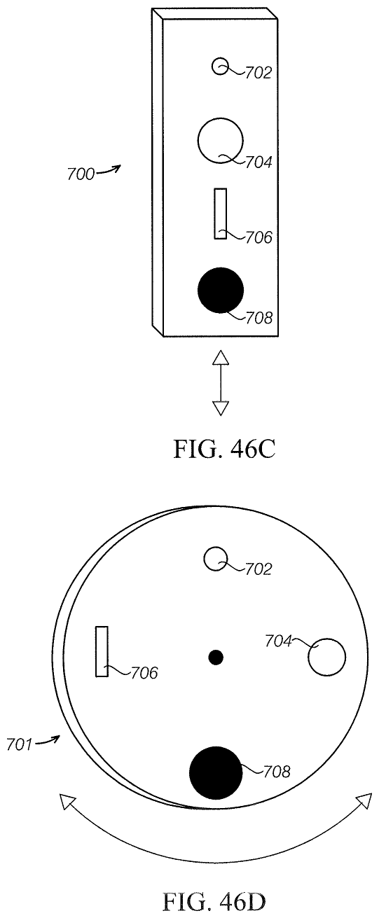

FIGS. 46A-46D illustrate embodiments of modules with multiple lenses that can be used with the adapters described herein. FIGS. 46A and 46C illustrate a module 700 with a small aperture lens 702, large aperture lens 704, slit lamp 706, and blue filter 710. The module 700 can move along the y-axis 156 to position the desired small aperture lens 702, large aperture lens 704, slit lamp 706, or blue filter 710 in front of the light source 142. FIGS. 46B and 46D illustrate a module 701 with a circular shape including a small aperture lens 702, large aperture lens 704, slit lamp 706, and blue filter 710. The module 701 can be rotated to position the desired small aperture lens 702, large aperture lens 704, slit lamp 706, or blue filter 710 in front of the light source 142. The modules 700, 701 can be removable.

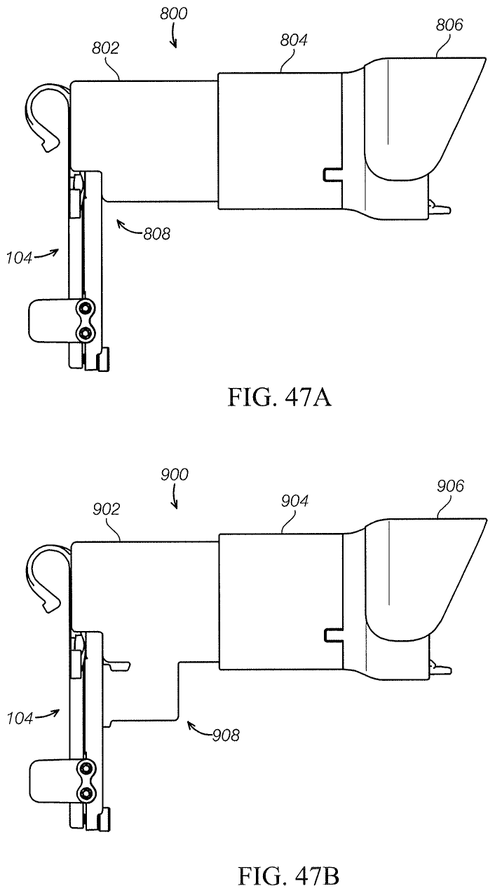

FIG. 47A illustrates an adapter 104 with a posterior portion 800 having an integral telescoping optical pathway enclosure. The posterior portion 800 includes a first section 802, second section 804, and optional visor 806 that adds additional protection from overhead or ambient light. The second section can removably receive the ophthalmoscopy lens 110 or come with the ophthalmoscopy lens 110 built into the second section 804. The second section 804 can move relative to the first section 802 to adjust the length between the anterior adapter 104 and the ophthalmoscopy lens 110 (not shown). The illustrated posterior portion 800 includes a connection element 808 configured to removably engage with the anterior adapter 104. The illustrated posterior portion 800 includes a magnet to secure the posterior portion 800 relative to the anterior adapter 104. The magnets can be designed to engage and line up the posterior portion 800 with the anterior adapter 104, with optional grooves one or both the posterior portion 800 and the anterior adapter 104 that facilitate proper optical alignment.

FIG. 47B illustrates an adapter 104 with a posterior portion 900 having an integral telescoping optical pathway enclosure. The posterior portion 900 includes a first section 902, second section 904, and optional enclosure 906. The second section can removably receive the ophthalmoscopy lens 110 or come with the ophthalmoscopy lens 110 (not shown) built into the second section 904. The second section 904 can move relative to the first section 902 to adjust the length between the anterior adapter 104 and the ophthalmoscopy lens 110. The illustrated posterior portion 900 includes a connection element 908 configured to removably engage with the anterior adapter 104. The illustrated connection element 908 includes a base that can be removably received by a complementary structure, such as the complementary mating structure 162.

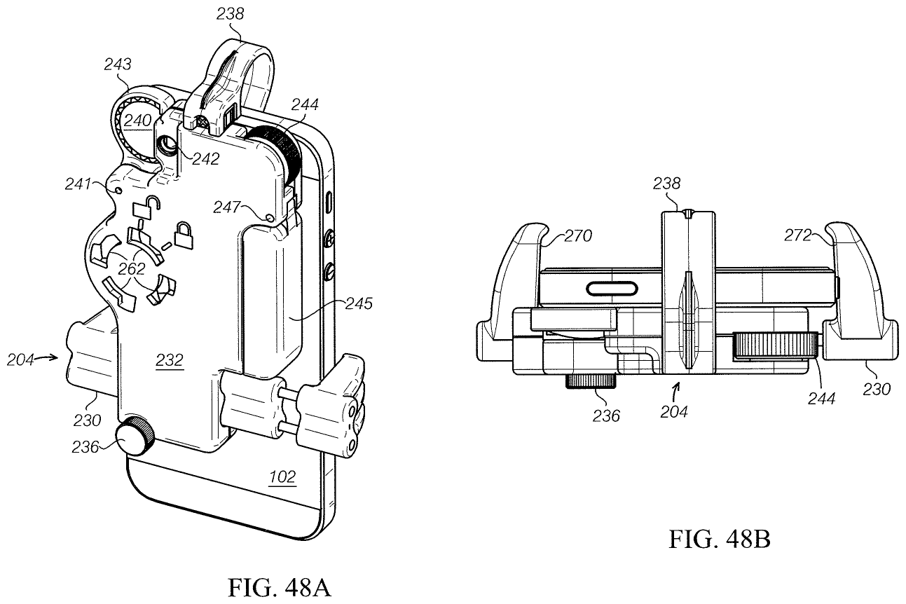

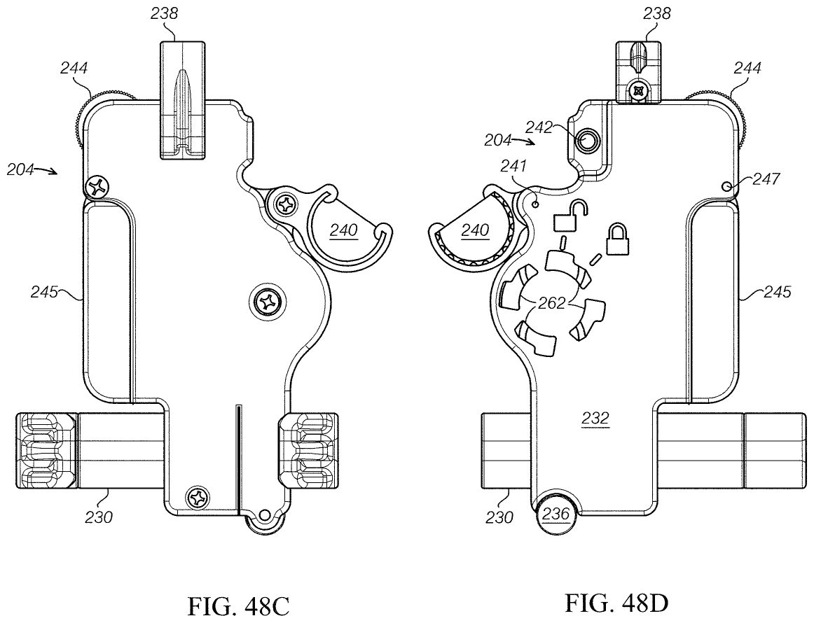

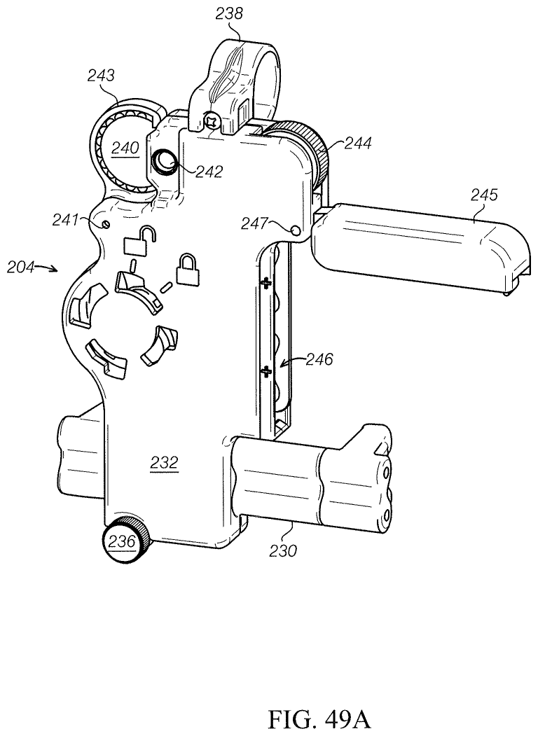

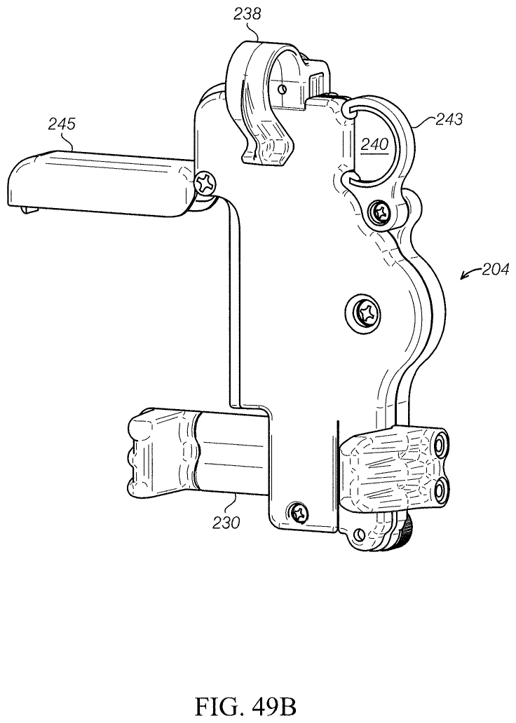

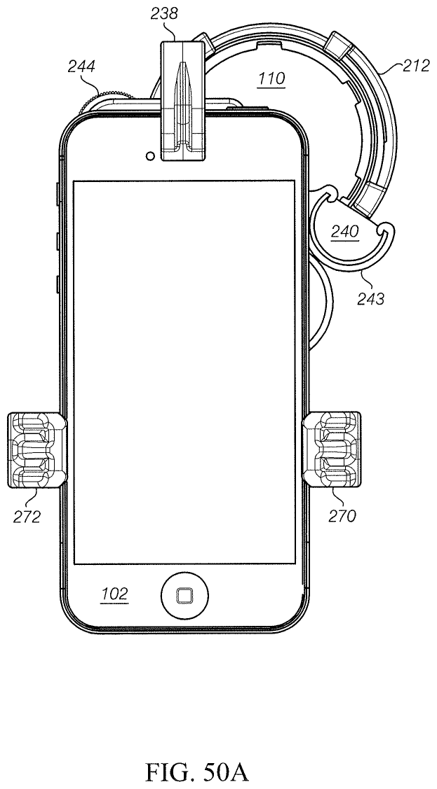

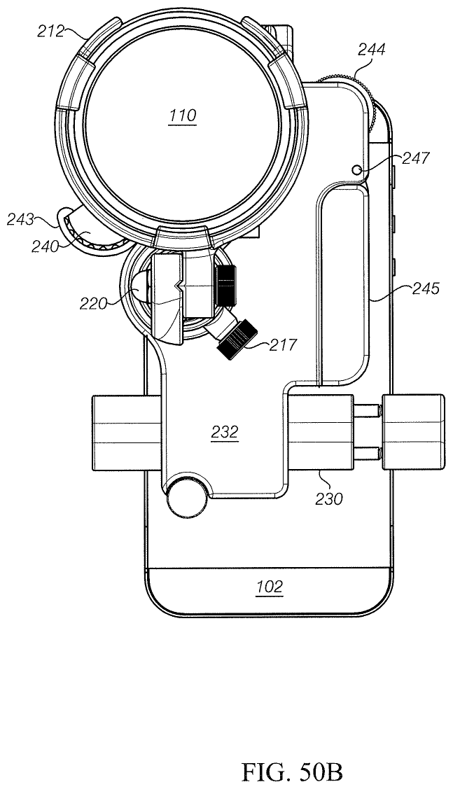

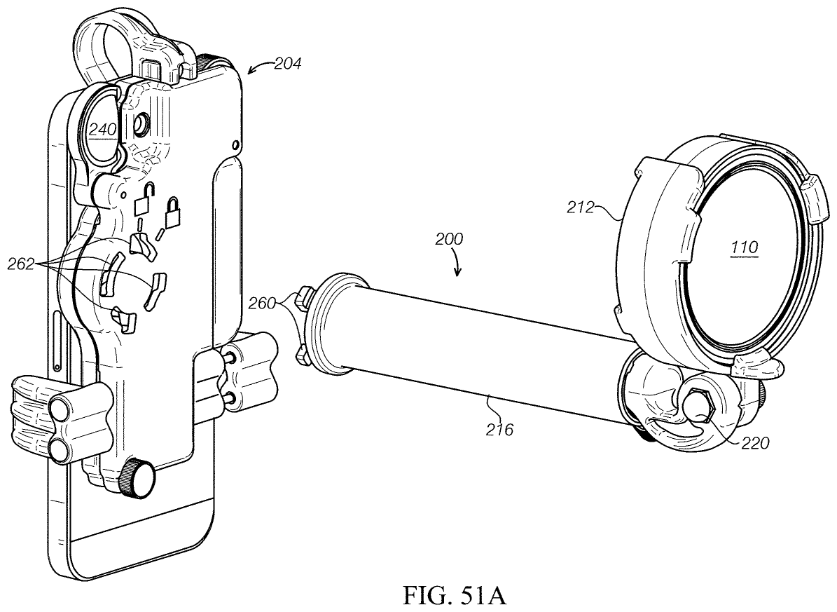

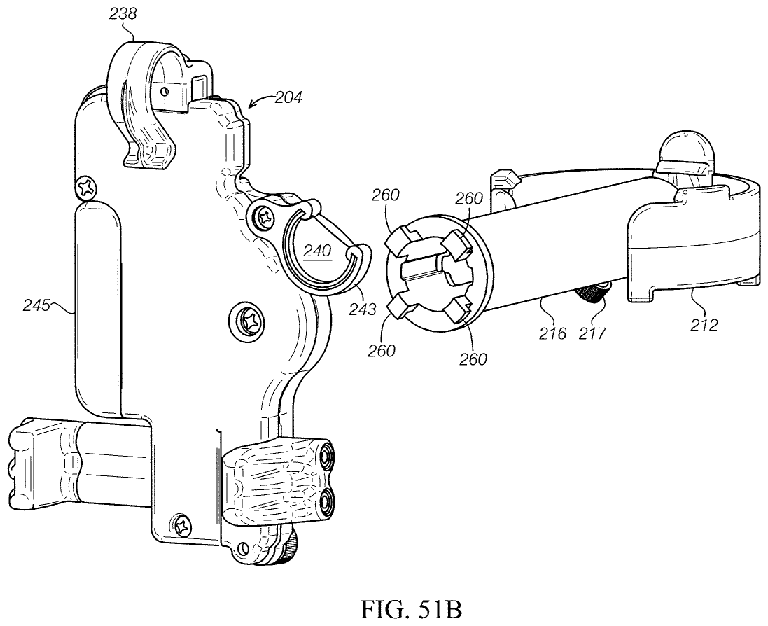

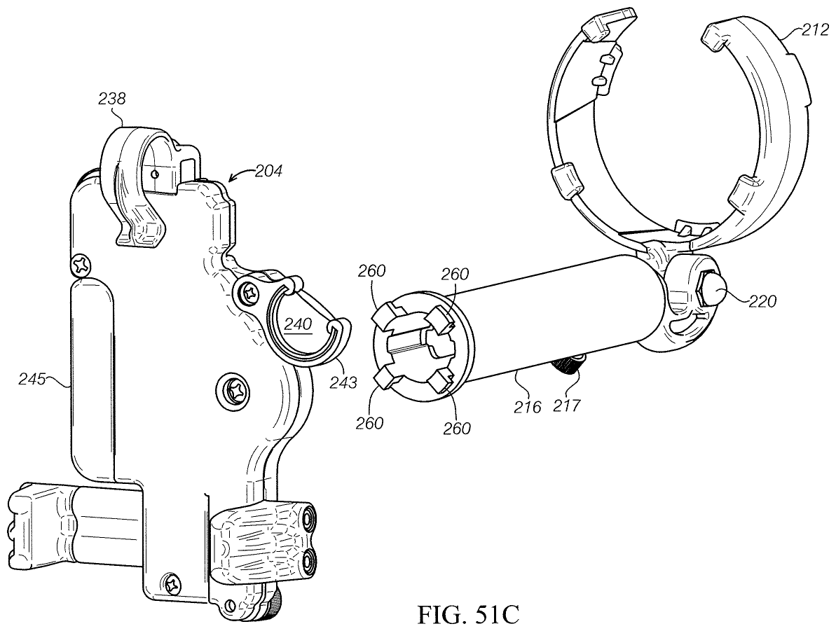

FIGS. 48A-48D, 49A-49B, 50A-50B, and 51A-51C illustrate additional views of embodiments of the adapter 200 described herein. The adapter 200 includes an anterior adapter portion 204 and a removably engageable posterior portion 206. The adapter 200 is generally similar to the adapter 100 but with some modifications to the shape of the base 232 and other features of the adapter 200. The anterior adapter portion body 232 can be secured relative to the horizontal clamp 230 by a locking mechanism 236, such as the illustrated adjustable screw. The horizontal clamp 230 includes a first clamp surface 270 and a second clamp surface 272 adapted to engage with the hand held computer device 102. The illustrated adapter 200 includes a third engagement surface or vertical contact point 238, illustrated with a hook type configuration to hold the hand held computer device 200 flush with the anterior adapter portion 204. The illustrated anterior adapter portion 204 also includes a macro lens 240, macro lens holder 243, lens holder hinge 241, light source 242, and light source dial control 244. The illustrated light source 242 is a LED. The lens holder 243 can be adapted to receive other types of lenses. The anterior adapter portion 204 includes a battery door 245, battery compartment 246, and battery door hinge 247. FIGS. 49A and 49B illustrate the battery door 245 in an open position showing the battery compartment 246.

The posterior portion 206 includes a lens 110 (such as an ophthalmoscopy lens) and lens holder 212. The posterior portion 206 can include a base shaft 216 and telescoping shaft (shown in a retracted position) configured to move relative to one another to modify the length of the posterior portion 206. The adjustable screw 220 can also be configured to lock the movement of the telescoping shaft relative to the base shaft 216 in some embodiments. A telescoping section locking mechanism 217, which is illustrated as a thumb screw can be used to adjust the length of the posterior section 206 and restrict relative movement between the base shaft 216 and telescoping section. The illustrated posterior portion 206 includes a male engagement structure 260 shown with four prongs. The male engagement structures is configured to engage with a complementary female mating structure 262 of the anterior adapter portion 204. The prongs can engage with the complementary structure and be rotated to lock into position.

The present application focuses on the workflow for providing eye care to a patient; however, the workflows described herein can also be applied to dermatology and other health care practice areas. For example, the non-ophthalmologist can be replaced by a non-dermatologist and the ophthalmologist can be replaced by a dermatologist. The images of the patient can be images of the skin or epidermis instead of the eye of the patient. The images of the skin of the patient can be obtained by the healthcare provider or non-dermatologist and then sent to the dermatologist for an assessment and/or referral. Follow up appointments can be scheduled for the patient based on the assessment done by the dermatologist and the severity or urgency needed to treat any potential issues provided in the assessment done by the dermatologist.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for obtaining an image of a retina of a patient, the method comprising:
analyzing an image obtained by a camera of a mobile device to look for a contour of an indirect lens along an optical axis of the camera of the mobile device;
upon detection of the contour of the indirect lens, determining whether an image of the retina is present in the indirect lens;
analyzing the image of the retina to determine one or more predetermined quality parameters associated with the image of the retina obtained by the camera of the mobile device; and
providing an indication to a user of the mobile device that corresponds to the one or more predetermined quality parameters associated with the image of the retina obtained by the camera of the mobile device.

2. The method of claim 1, further comprising: saving the image of the retina if a predetermined quality threshold is met by the one or more quality parameters associated with the image of the retina obtained by the camera of the mobile device.

3. The method of any one of the preceding claims, further comprising: applying a mask to an area of the image outside of the contour of the indirect lens to create a masked image of the retina.

4. The method of claim 3, further comprising: displaying the masked image of the retina on a display of the mobile device.

5. The method of any one of the preceding claims, further comprising: analyzing a plurality of images of the retina and saving a plurality of images of the retina that meet a predetermined quality threshold.

6. The method of claim 5, further comprising: saving the plurality of images of the retina that meet the predetermined quality threshold.

7. The method of any of claims 5-6, wherein the plurality of images of the retina are obtained from a video feed.

8. The method of any of claims 5-7, wherein the plurality of images of the retina are obtained from a multiple pictures taken by the camera of the mobile device.

9. The method of any of claims 5-8, wherein the plurality of images of the retina that meet the predetermined quality threshold includes a predetermined number of images of the retina.

10. The method of claim 9, wherein the predetermined number of images is 10 or less images of the retina.

11. The method of any of claims 9-10, wherein the predetermined number of images is set by a user of the mobile imaging device.

12. The method of any one of the preceding claims, wherein the one or more predetermined quality parameters associated with the image of the retina include one or more of: glare, exposure, a comparison with an ideal retina image, focus, and lighting.

13. The method of any one of the preceding claims, wherein the lens contour has a substantially circular shape.

14. The method of any one of the preceding claims, further comprising: displaying an inverted image of the retina from the indirect lens on a display of the mobile device.

15. A method of displaying an image of a retina on a mobile device comprising:
receiving an image obtained by a camera of a mobile device of an indirect lens along an optical axis of the camera of the mobile device, the image of the indirect lens including an image of a retina of a patient;
inverting the image of the indirect lens to form an inverted image of the indirect lens and the retina; and
displaying the inverted image of the indirect lens and retina on a display of the mobile device.

16. The methods of any one of the preceding claims, wherein the indirect lens has a size of about 10 D to 90 D.

17. The methods of any one of the preceding claims, wherein the indirect lens is selected from the group consisting of: 14 D, 20 D, 22 D, 28 D, 30 D, 40 D, or 54 D, 60, 66, and 90 D.

18. The methods of any one of the preceding claims, wherein the indirect lens is removably engaged with a lens mount of a lens adapter.

19. The method of claim 18, wherein the lens adapter is removably engaged with the mobile device.

20. The method of any one of the preceding claims, wherein the lens adapter includes a telescoping arm engaged with the lens mount and a base of the lens adapter engaged with the mobile device.

21. The method of any one of the preceding claims, further comprising: varying an intensity of a variable intensity light source of the lens adapter engaged with the mobile device to illuminate the retina.

22. The method of any one of the preceding claims, wherein any of the steps are performed by a mobile application on the mobile device.

23. The method of any one of the preceding claims, wherein the mobile device is a hand held computer device, smartphone, tablet computer, or mobile imaging device.

24. The method of any one of the preceding claims, further comprising: automatically centering the image of the retina on a display of the mobile device.

25. The method of any one of the preceding claims, further comprising: automatically focusing the camera of the mobile device on the image of the retina.

26. The method of any one of the preceding claims, further comprising: presenting the images of the retina that meet a predetermined quality threshold on a display of the mobile device.

27. The method of any one of the preceding claims, further comprising: sending one or more of the images of the retina that meet a predetermined quality threshold to an electronic medical record (EMR) or electronic health record (EHR) of the patient.

28. The method of claim 27, further comprising: automatically saving the one or more images of the retina to the EMR or EHR of the patient.

29. The method of any one of the preceding claims, further comprising: analyzing a plurality of images of the retina, applying one or more digital image processing techniques to the plurality of the images of the retina, and forming a combined image of the retina based on the plurality of images of the retina and the applied one or more digital image processing techniques.

30. A method for obtaining an image of an eye of a patient, the method comprising:
receiving an image of an anterior segment of an eye of a patient with a camera of a mobile device through a lens of a lens adapter engaged with the mobile device;
analyzing the image of the anterior segment of the eye to determine one or more quality parameters associated with the image of the anterior segment of the eye; and
providing an indication to a user of the mobile device that corresponds to the one or more quality parameters associated with the image of the anterior segment obtained by the camera of the mobile device.

31. The method of claim 30, further comprising: saving the image of the anterior segment if a predetermined quality threshold is met by the one or more quality parameters associated with the image of the anterior segment obtained by the camera of the mobile device.

32. The method of any one of claims 30-31, further comprising: varying an intensity of a variable intensity light source of the lens adapter engaged with the mobile device to illuminate the anterior segment of the eye.

33. The method of any one of claims 30-32, wherein any of the steps are performed by a mobile application on the mobile device.

34. The method of any one of claims 30-33, wherein the mobile device is a hand held computer device, smartphone, tablet computer, or mobile imaging device.

35. The method of any one of claims 30-34, wherein the lens is a macro lens.

36. The method of any one of claims 30-35, wherein the lens adapter includes: a body, a clamp configured to engage with the mobile device at a first location and a second location, a lens holder engaged with a macro lens movable between a first position in the optical axis of the camera of the mobile device and a second position outside of the optical axis of the camera of the mobile device, an adjustable light source with a light axis parallel to a macro lens optical axis, a third engagement surface configured to slidably engage with the mobile device at a third location, wherein the clamp defines an axis and the body of the anterior adapter portion is configured to move along the axis of the clamp.

37. The method of claim 36, wherein the lens adapter further comprises: a complementary surface of the body configured to reversibly engage with a base section of a posterior portion, the posterior portion comprising: the base section configured to reversibly engage with the complementary surface of the body of the lens adapter, a telescoping section movable relative to the base section, and a lens holder engaged with a distal end of the telescoping section configured to removably engage with an indirect lens, the base section configured to removably engage with the body of the anterior adapter portion to form an optical axis between the ophthalmoscopy lens and the camera of the mobile device.

38. The method of any one of claims 30-37, further comprising: automatically focusing the camera of the mobile device on the image of the anterior segment of the eye.

39. The method of any one of claims 30-38, further comprising: presenting the image of the anterior segment of the eye that meet the predetermined quality threshold on a display of the mobile device.

40. The method of any one of claims 30-39, further comprising: sending one or more of the images of the anterior segment of the eye that meet the predetermined quality threshold to an electronic medical record (EMR) or electronic health record (EHR) of the patient.

41. The method of claim 40, further comprising: automatically saving the one or more of the images of the anterior segment of the eye to the EMR or EHR of the patient.

42. The method of any one of the preceding claims, further comprising: saving the image to a cloud storage network in a HIPAA compliant manner.

43. The method of claim 42, wherein the image is encrypted.

44. The method of any one of the preceding claims, wherein the non-ophthalmologist is a primary care doctor, an emergency room doctor, an optometrists, or an urgent care doctor.

45. The method of any one of claims 30-44, further comprising: receiving a plurality of images of the anterior segment of the eye of a patient with the camera of the mobile device through the lens of the lens adapter engaged with the mobile device.

46. The method of claim 46, further comprising: analyzing the plurality of images of the anterior segment of the eye of the patient, applying one or more digital image processing techniques to the plurality of the images of the anterior segment of the eye of the patient, and forming a combined image of the anterior segment based on the plurality of images of the anterior segment of the eye of the patient and the applied one or more digital image processing techniques.

47. A method comprising:
receiving images of a portion of an eye of a patient obtained by a non-ophthalmologist with a camera of a mobile device engaged with a lens adapter through a mobile application;
sending the images of the portion of the eye of the patient to an ophthalmologist through the mobile application; and
receiving notes on the image of the portion of the eye of the patient from the ophthalmologist through the mobile application.

48. The method of claim 47, wherein the non-ophthalmologist is a primary care doctor, an emergency room doctor, an optometrists, or an urgent care doctor.

49. The method of any one of claims 47-48, wherein the ophthalmologist is in a referring network with the non-ophthalmologist.

50. The method of any one of claims 47-48, wherein the ophthalmologist is in a referring network of a mobile application database.

51. The method of any one of claims 47-50, further comprising: receiving a referral recommendation from the ophthalmologist for an emergency appointment with an ophthalmologist through the mobile application.

52. The method of any one of claims 47-51, further comprising: receiving a referral recommendation from the ophthalmologist for a non-emergency appointment with an ophthalmologist through the mobile application.

53. The method of any one of claims 47-52, further comprising: receiving an ophthalmology assessment from the ophthalmologist through the mobile application including one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, other eye examination data associated with the patient, and one or more comments from the ophthalmologist.

54. The method of claim 53, further comprising: automatically generating a report including the ophthalmology assessment from the ophthalmologist.

55. The method of any one of claims 47-54, further comprising: automatically generating a reimbursement form for the ophthalmologist with billing codes based on the ophthalmology assessment.

56. The method of any one of claims 47-55, wherein the image of the portion of the eye of the patient includes an image of a retina of the patient obtained with an indirect lens engaged with the lens adapter.

57. The method of claim 56, wherein the image of the retina is obtained using any of the methods of claims 1-29.

58. The method of any one of claims 47-57, wherein the image of the portion of the eye of the patient includes an image of an anterior segment of the eye of the patient obtained with a macro lens of the lens adapter.

59. The method of claim 58, wherein the image of the anterior segment is obtained using any of the methods of claims 30-46.

60. A method comprising:
presenting a non-ophthalmologist with a patient in need of an eye examination or acute care of the eye;
conducting an examination of the patient by the non-ophthalmologist using a mobile device and a lens adapter removably engaged with the mobile device and a mobile application to generate a patient examination data within the mobile application;
sending the patient examination data to an ophthalmologist for review;
receiving a patient assessment from the ophthalmologist based on the patient examination data; and
sending the patient assessment to the non-ophthalmologist.

61. The method of claim 60, wherein the non-ophthalmologist is a primary care doctor, an emergency room doctor, an optometrists, or an urgent care doctor.

62. The method of any one of claims 60-61, wherein the ophthalmologist is in a referring network with the non-ophthalmologist.

63. The method of any one of claims 60-62, wherein the ophthalmologist is in a referring network of a mobile application database.

64. The method of any one of claims 60-63, further comprising: receiving a referral recommendation from the ophthalmologist for an emergency appointment with an ophthalmologist through the mobile application.

65. The method of any one of claims 60-64, further comprising: receiving a referral recommendation from the ophthalmologist for a non-emergency appointment with an ophthalmologist through the mobile application.

66. The method of any one of claims 64-65, further comprising: receiving through the mobile application an assessment from the emergency appointment with the ophthalmologist or an assessment from the non-emergency appointment with the ophthalmologist.

67. The method of any one of claims 64-65, further comprising: sending a notification to the mobile application after the patient sees the ophthalmologist for the emergency appointment or non-emergency appointment.

68. The method of any one of claims 60-67, wherein the patient examination data includes one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, and other eye examination data associated with the patient.

69. The method of any one of claims 60-68, wherein the patient assessment from the ophthalmologist includes one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, other eye examination data associated with the patient, and one or more comments from the ophthalmologist.

70. The method of claim 69, further comprising: automatically generating a report including the patient assessment from the ophthalmologist.

71. The method of any one of claims 60-70, further comprising: automatically generating a reimbursement form for the ophthalmologist with billing codes based on the patient assessment.

72. The method of any one of claims 60-71, further comprising: automatically populating an electronic health record (EHR) of the patient with the patient examination data and the patient assessment.

73. The method of any one of claims 60-72, wherein the image of the portion of the eye of the patient includes an image of a retina of the patient obtained with an indirect lens engaged with the lens adapter.

74. The method of claim 73, wherein the image of the retina is obtained using any of the methods of claims 1-29.

75. The method of any one of claims 60-74, wherein the image of the portion of the eye of the patient includes an image of an anterior segment of the eye of the patient obtained with a macro lens of the lens adapter.

76. The method of claim 75, wherein the image of the anterior segment is obtained using any of the methods of claims 30-46.

77. A method comprising:
creating an order for an eye examination of a patient;
sending the order for the eye examination of the patient to a mobile application;
matching a patient ID of the patient to an electronic health record (EHR) for the patient;
receiving a patient data point from a non-ophthalmologist using the mobile application and a lens adapter engaged with a mobile device running the mobile application;
sending the patient data point to the electronic health record; and
automatically populating the electronic health record with the patient data point.

78. The method of claim 77, wherein the non-ophthalmologist is a primary care doctor, an emergency room doctor, an optometrists, or an urgent care doctor.

79. The method of any one of claims 77-78, further comprising: sending instructions for the eye examination of the patient through the mobile device to the non-ophthalmologist.

80. The method of any one of claims 77-79, wherein the patient examination data includes one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, and other eye examination data associated with the patient.

81. The method of any one of claims 77-80, wherein the image of the portion of the eye of the patient includes an image of a retina of the patient obtained with an indirect lens engaged with the lens adapter.

82. The method of claim 81, wherein the image of the retina is obtained using any of the methods of claims 1-29.

83. The method of any one of claims 77-82, wherein the image of the portion of the eye of the patient includes an image of an anterior segment of the eye of the patient obtained with a macro lens of the lens adapter.

84. The method of claim 83, wherein the image of the anterior segment is obtained using any of the methods of claims 30-46.

85. A method comprising:
receiving a patient data point including eye examination data collected with a mobile application with a lens adapter engaged with a mobile device running the mobile application;
receiving an assessment of the patient data point done by an ophthalmologist with the mobile application;
receiving an electronic signature from the ophthalmologist;
automatically generating billing codes that correspond to the patient data point and the assessment of the patient data point;
automatically generating a report including the billing codes, patient data point, and the assessment of the patient data point; and
submitting the report for reimbursement.

86. The method of claim 85, wherein the patient data point is collected by a non-ophthalmologist.

87. The method of claim 86, wherein the non-ophthalmologist is a primary care doctor, an emergency room doctor, an optometrists, or an urgent care doctor.

88. The method of any one of claims 85-87, wherein the patient examination data includes one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, and other eye examination data associated with the patient.

89. The method of any one of claims 85-88, wherein the assessment of the patient data point done by the ophthalmologist includes one or more of: a family history, a patient symptom, a patient medication, an image of the retina, an image of an anterior segment of the eye, a visual acuity of the patient, an intraocular pressure of the patient, an afferent defect of the patient, a corneal abrasion of the patient, other eye examination data associated with the patient, and one or more comments from the ophthalmologist.

90. The method of any one of claims 85-89, wherein the image of the portion of the eye of the patient includes an image of a retina of the patient obtained with an indirect lens engaged with the lens adapter.

91. The method of claim 90, wherein the image of the retina is obtained using any of the methods of claims 1-29.

92. The method of any one of claims 85-91, wherein the image of the portion of the eye of the patient includes an image of an anterior segment of the eye of the patient obtained with a macro lens of the lens adapter.

93. The method of claim 92, wherein the image of the anterior segment is obtained using any of the methods of claims 30-46.

94. A system comprising:
a mobile imaging device with a camera, the mobile imaging device configured to run a computer executable code comprising any of the steps of any of the preceding claims; and
a lens adapter configured to removably engage with the mobile imaging device.

95. The system of claim 94, wherein the lens adapter is any of the lens adapters in claims 96-101.

96. The methods of any of the preceding claims, wherein the adapter comprises:
an adapter configured to engage with a hand held computer device with a camera having an optical axis comprising:
an anterior adapter portion comprising: a body, a clamp configured to engage with the hand held computer device at a first location and a second location, a lens holder engaged with a macro lens movable between a first position in the optical axis of the camera and a second position outside of the optical axis of the camera, an adjustable light source with a light axis parallel to a macro lens optical axis, a third engagement surface configured to slidably engage with the hand held computer device at a third location, and a complementary surface of the body configured to reversibly engage with a base section of a posterior portion, wherein the clamp defines an axis and the body of the anterior adapter portion is configured to move along the axis of the clamp; and
the posterior portion comprising: the base section configured to reversibly engage with the complementary surface of the body of the anterior adapter portion, a telescoping section movable relative to the base section, and a lens holder engaged with a distal end of the telescoping section configured to removably engage with an ophthalmoscopy lens, the base section configured to removably engage with the body of the anterior adapter portion to form an optical axis between the ophthalmoscopy lens and the camera of the hand held computer device.

97. The adapter of claim 96, further comprising: a removable enclosure configured to removably engage with the posterior portion.

98. The adapter of claim 97, wherein the removable enclosure includes a clamping mechanism to engage with the posterior portion.

99. The adapter of any of claims 97-98, the removable enclosure further comprising: a telescoping portion configured to adjust a length of the removable cover.

100. The adapter of any one of claims 97-99, the removable enclosure further comprising a proximal portion with an opening to accommodate the camera of the hand held computer device and the light source of the anterior adapter portion and a distal section to engage with the lens holder.

101. The adapter of any one of claims 97-100, wherein the removable enclosure is adapted to encase the optical pathway between the camera and the lens holder.
